SEQUENCE LISTING (37 sequences)

SEQ ID NO: 1 - DNA, 74 bp, Artificial Sequence
  Synthetic Polynucleotide
aattgtgagcggataacaattgacattgtgagcggataacaagatactgagcacatcagcaggacgcactgacc

SEQ ID NO: 2 - DNA, 74 bp, Artificial Sequence
  Synthetic Polynucleotide
tccctatcagtgatagagattgacatccctatcagtgatagagatactgagcacatcagcaggacgcactgacc

SEQ ID NO: 3 - DNA, 53 bp, Artificial Sequence
  Synthetic Polynucleotide
acctgtaggatcgtacaggtttacgcaagaaaatggtttgttatagtcgaata

SEQ ID NO: 4 - DNA, 49 bp, Artificial Sequence
  Synthetic Polynucleotide
taacaccgtgcgtgttgactattttacctctggcggtgataatggttgc

SEQ ID NO: 5 - DNA, 247 bp, Artificial Sequence
  Synthetic Polynucleotide
acgcccgtgatcctgatcaccggctatccggacgaaaacatctcgacccgggccgccgaggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggag

SEQ ID NO: 6 - DNA, 82 bp, Artificial Sequence
  Synthetic Polynucleotide
atgcgcacccttagcgagaggtttatcattaaggtcaacctctggatgttgtttcggcatcctgcattgaatctgagttact

SEQ ID NO: 7 - DNA, 86 bp, Artificial Sequence
  Synthetic Polynucleotide
gtcagaaaaaacgggtttcctggttggctcggagagcatcaggcgatgctctccgttccaacaaggaaaacagacagtaactcaga

SEQ ID NO: 8 - DNA, 131 bp, Artificial Sequence
  Synthetic Polynucleotide
gtcagaaaaaacgggtttcctgaattccaacatggatgctgatttatatgggtataaatgggcccgcgataatgtcgggcaatcaggtgcgacaatctatcggaattcaggaaaacagacagtaactcaga

SEQ ID NO: 9 - DNA, 134 bp, Artificial Sequence
  Synthetic Polynucleotide
gtcagaaaaaacgggtttcctgaattccagctaatttccgcgctcggcaagaaagatcatgcctaatgaatcgattgccgctcactggggaccaaagcagtttccgaattcaggaaaacagacagtaactcaga

SEQ ID NO: 10 - DNA, 134 bp, Artificial Sequence
  Synthetic Polynucleotide
gtcagaaaaaacgggtttcctgaattccagctaatttccgcgctcggcaagaaagatcatgccctcttgatcgattgccgctcactggggaccaaagcagtttccgaattcaggaaaacagacagtaactcaga

SEQ ID NO: 11 - DNA, 134 bp, Artificial Sequence
  Synthetic Polynucleotide
gtcagaaaaaacgggtttcctgaattcacccaacttaatcgccttgcagcacatccccctttcgccagctggcgtaatagcgaagaggcccgcaccgatcgccctgaattcaggaaaacagacagtaactcaga

SEQ ID NO: 12 - DNA, 963 bp, Artificial Sequence
  Synthetic Polynucleotide
atgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgacctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaa

SEQ ID NO: 13 - DNA, 786 bp, Artificial Sequence
  Synthetic Polynucleotide
atgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 14 - DNA, 747 bp, Artificial Sequence
  Synthetic Polynucleotide
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccgagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggcgctgcaaacgacgaaaactacgctttagtagcttaa

SEQ ID NO: 15 - DNA, 1754 bp, Artificial Sequence
  Synthetic Polynucleotide
gtggctagttttcaatcatttgggataccaggacagctggaagtcatcaaaaaagcacttgatcacgtgcgagtcggtgtggtaattacagatcccgcacttgaagataatcctattgtctacgtaaatcaaggctttgttcaaatgaccggctacgagaccgaggaaattttaggaaagaactgtcgcttcttacaggggaaacacacagatcctgcagaagtggacaacatcagaaccgctttacaaaataaagaaccggtcaccgttcagatccaaaactacaaaaaagacggaacgatgttctggaatgaattaaatattgatccaatggaaatagaggataaaacgtattttgtcggtattcagaatgatatcaccgagcaccagcagacccaggcgcgcctccaggaactgcaatccgagctcgtccacgtctccaggctgagcgccatgggcgaaatggcgtccgcgctcgcgcacgagctcaaccagccgctggcggcgatcagcaactacatgaagggctcgcggcggctgcttgccggcagcagtgatccgaacacaccgaaggtcgaaagcgccctggaccgcgccgccgagcaggcgctgcgcgccggccagatcatccggcgcctgcgcgacttcgttgcccgcggcgaatcggagaagcgggtcgagagtctctccaagctgatcgaggaggccggcgcgctcgggcttgccggcgcgcgcgagcagaacgtgcagctccgcttcagtctcgatccgggcgccgatctcgttctcgccgaccgggtgcagatccagcaggtcctggtcaacctgttccgcaacgcgctggaagcgatggctcagtcgcagcgacgcgagctcgtcgtcaccaacacccccgccgccgacgacatgatcgaggtcgaagtgtccgacaccggcagcggtttccaggacgacgtcattccgaacctgtttcagactttcttcaccaccaaggacaccggcatgggcgtgggactgtccatcagccgctcgatcatcgaagctcacggcgggcgcatgtgggccgagagcaacgcatcgggcggggcgaccttccgcttcaccctcccggcagccgacgagatgataggaggtctagcatgacgaccaagggacatatctacgtcatcgacgacgacgcggcgatgcgggattcgctgaatttcctgctggattctgccggcttcggcgtcacgctgtttgacgacgcgcaagcctttctcgacgccctgccgggtctctccttcggctgcgtcgtctccgacgtgcgcatgccgggccttgacggcatcgagctgttgaagcggatgaaggcgcagcaaagcccctttccgatcctcatcatgaccggtcacggcgacgtgccgctcgcggtcgaggcgatgaagttaggggcggtggactttctggaaaagcctttcgaggacgaccgcctcaccgccatgatcgaatcggcgatccgccaggccgagccggccgccaagagcgaggccgtcgcgcaggatatcgccgcccgcgtcgcctcgttgagccccagggagcgccaggtcatggaagggctgatcgccggcctttccaacaagctgatcgcccgcgagtacgacatcagcccgcgcaccatcgaggtgtatcgggccaacgtcatgaccaagatgcaggccaacagcctttcggagctggttcgcctcgcgatgcgcgccggcatgctcaacgat

SEQ ID NO: 16 - DNA, 816 bp, Artificial Sequence
  Synthetic Polynucleotide
atgagccatattcaacgggaaacgtcttgctcgaggccgcgattaaattccaacatggatgctgatttatatgggtataaataatagcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 17 - DNA, 3075 bp, Artificial Sequence
  Synthetic Polynucleotide
atgaccatgattacggattcactggccgtcgttttacaacgtcgtgactgggaaaaccctggcgttacccaacttaatcgccttgcagcacatccccctttctaatagtggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 18 - DNA, 2005 bp, Artificial Sequence
  Synthetic Polynucleotide
atgcgcacccttagcgagaggtttatcattaaggtcaacctctggatgttgtttcggcatcctgcattgaatctgagttactgtctgttttcctgaattccgatagattgtcgcacctgattgcccgacattatcgcgggcccatttatacccatataaatcagcatccatgttggaattcaggaaacccgttttttctgacgtaagggtgcgcaactttcatgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgacctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaaggatccggttgatattattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 19 - DNA, 60 bp, Artificial Sequence
  Synthetic Polynucleotide
gcgatatccattttcgcgaatccggagtgtaagaagagctcctgactccccgtcgtgtag

SEQ ID NO: 20 - DNA, 60 bp, Artificial Sequence
  Synthetic Polynucleotide
gaccgcagaacaggcagcagagcgtttgcgcgcagtcagcgatatccattttcgcgaatc

SEQ ID NO: 21 - DNA, 60 bp, Artificial Sequence
  Synthetic Polynucleotide
cggctgaccatcgggtgccagtgcgggagtttcgtgacgtcgttaagccagccccgacac

SEQ ID NO: 22 - DNA, 60 bp, Artificial Sequence
  Synthetic Polynucleotide
actaccatccctgcgttgttacgcaaagttaacagtcggtacggctgaccatcgggtgcc

SEQ ID NO: 23 - DNA, 90 bp, Artificial Sequence
  Synthetic Polynucleotide
cgcgattaaattccaacatggatgctgatttatatgggtataaataatagcgcgataatgtcgggcaatcaggtgcgacaatctatcgat

SEQ ID NO: 24 - DNA, 72 bp, Artificial Sequence
  Synthetic Polynucleotide
caacttaatcgccttgcagcacatccccctttctaatagtggcgtaatagcgaagaggcccgcaccgatcgc

SEQ ID NO: 25 - DNA, 41 bp, Artificial Sequence
  Synthetic Polynucleotide
gatatatacgagatatgccgctgctctcaccttatctgcac

SEQ ID NO: 26 - DNA, 41 bp, Artificial Sequence
  Synthetic Polynucleotide
gtgcagataaggtgagagcagcggcatatctcgtatatatc

SEQ ID NO: 27 - DNA, 20 bp, Artificial Sequence
  Synthetic Polynucleotide
aatacgcaaaccgcctctcc

SEQ ID NO: 28 - DNA, 20 bp, Artificial Sequence
  Synthetic Polynucleotide
cggcggattgaccgtaatgg

SEQ ID NO: 29 - DNA, 86 bp, Artificial Sequence; identical to SEQ ID NO: 7
  Synthetic Polynucleotide

SEQ ID NO: 30 - DNA, 12 bp, Artificial Sequence
  Synthetic Polynucleotide
tgcgcaccctta

SEQ ID NO: 31 - DNA, 13 bp, Artificial Sequence
  Synthetic Polynucleotide
cgcgnnnnnattt

SEQ ID NO: 32 - DNA, 10 bp, Artificial Sequence
  Synthetic Polynucleotide
atgcagctta

SEQ ID NO: 33 - DNA, 10 bp, Artificial Sequence
  Synthetic Polynucleotide
accgtagatc

SEQ ID NO: 34 - DNA, 30 bp, Artificial Sequence
  Synthetic Polynucleotide
catccccctttcgccagctggcgtaatagc

SEQ ID NO: 35 - DNA, 30 bp, Artificial Sequence
  Synthetic Polynucleotide
catccccctttctaatagtggcgtaatagc

SEQ ID NO: 36 - DNA, 30 bp, Artificial Sequence
  Synthetic Polynucleotide
tatgggtataaatgggctcgcgataatgtc

SEQ ID NO: 37 - DNA, 30 bp, Artificial Sequence
  Synthetic Polynucleotide
tatgggtataaataatagcgcgataatgtc

(19) United States
(12) Patent Application Publication
(10) Pub. No.: US 2017/0204399 A1
(43) Pub. Date: Jul. 20, 2017

(54) GENOMICALLY-ENCODED MEMORY IN LIVE CELLS

(71) Applicant: Massachusetts Institute of Technology, Cambridge, MA (US)

(72) Inventors: Timothy Kuan-Ta Lu, Cambridge, MA (US); Fahim Farzadfard, Boston, MA (US)

(73) Assignee: Massachusetts Institute of Technology, Cambridge, MA (US)

(21) Appl. No.: 15/324,487

(22) PCT Filed: Aug. 13, 2015
(86) PCT No.: PCT/US2015/045069

§ 371 Date: Jan. 6, 2017

Related U.S. Application Data
(60) Provisional application No. 62/037,679, filed on Aug. 15, 2014.
(60) Provisional application No. 62/066,184, filed on Oct. 20, 2014.

(51) Int. Cl. C12N 15/10; C12N 9/12; C12N 9/22; C12N 15/63

(52) U.S. Cl. CPC C12N 15/1024; C12N 15/635; C12N 9/1276; C12N 9/22; C12Y 207/07049

(57) ABSTRACT

Aspects of the present disclosure provide synthetic-biology platforms for in vivo genome editing, which enable the use of live cell genomes as “tape recorders” for long-term recording of event histories and analog memories.

### RELATED APPLICATIONS

This application claims the benefit under 35 U.S.C. §119(e) of U.S. provisional application No. 62/037,679, filed Aug. 15, 2014, and U.S. provisional application No. 62/066,184, filed Oct. 20, 2014, the disclosures of each of which are incorporated by reference herein in their entirety.

### FEDERALLY SPONSORED RESEARCH

This invention was made with Government support under Contract No. N00014-11-1-0725 awarded by the Office of Naval Research and under Grant No. DMR-0819762 awarded by the National Science Foundation. The Government has certain rights in the invention.

### FIELD OF THE INVENTION

Aspects of the present disclosure relate to the field of biological engineering.

### BACKGROUND OF THE INVENTION

Living cell populations constitute a rich resource for biological computation and memory. Cellular memory is a crucial aspect of many natural biological processes and is important for enabling sophisticated synthetic biology applications. Existing cellular memory relies on epigenetic switches or recombinase-based mechanisms, which are limited in scalability and recording capacity.

### SUMMARY OF THE INVENTION

The present disclosure, in some aspects, provides for the use of deoxyribonucleic acid (DNA) of living cell populations as genomic ‘tape recorders’ for the analog and multiplexed recording of event (e.g., long-term event) histories. Provided herein, in some embodiments, is a platform for generating single-stranded DNA (ssDNA) inside living cells in response to, for example, arbitrary transcriptional signals, such as chemical and non-chemical inducers (e.g., light). When co-expressed with a recombinase, these intracellularly expressed ssDNAs uniquely target specific genomic DNA sequences, resulting in precise mutations that accumulate in cell populations as a function of the magnitude and duration of the inputs (e.g., transcriptional signals). The approach as provided herein enables the memorization of inputs into genomic memory (e.g., long-lasting genomic memory) through in vivo genome editing and the reading of memory with a variety of strategies. Using this platform, the present disclosure demonstrates autonomous, long-term and multiplexable recording and resetting of event histories directly in the DNA of live cell populations and is applicable to a broad range of host cells. This platform for in vivo genome editing enables, inter alia, the use of live cell populations as long-term recorders for environmental and biomedical applications, the construction of cellular state machines, and enhanced genome engineering strategies.

Thus, some aspects of the present disclosure relate to scalable platforms that use genomic DNA for analog, rewritable, and/or multiplexed memory in live cell populations (FIG. 1A). These scalable platforms, referred to herein as SCRIBE (Synthetic Cellular Recorders Integrating Biological Events) platforms, enable in vivo recording of arbitrary inputs into DNA storage registers by converting transcriptional signals into ssDNAs. Instead of storing the digital absence or presence of inputs, these memory units can record the analog magnitude and time of exposure to inputs in the fraction of cells in a population that carry a specific mutation (FIG. 1B). Based on sequence homology, ssDNAs generated in live cells can be addressed to specific target loci in the genome where they are recombined and converted into permanent memory (FIG. 1C). These memory units can be readily reprogrammed, integrated with logic circuits, and decomposed into independent input, write and/or read operations.

Although aspects of the present disclosure relate to targeting mutations into functional genes to facilitate convenient functional and reporter assays, the present disclosure also contemplates natural or synthetic non-coding DNA segments for use in recording memory within genomic DNA. For example, by targeting genomic DNA such as ribosomal binding sites and transcriptional regulatory sequences, gene expression can be tuned quantitatively rather than just “ON” (e.g., expressed) or “OFF” (e.g., not expressed) A potential benefit of using synthetic DNA segments as memory registers is the ability to introduce mutations for memory storage that are neutral in terms of fitness costs.

Some aspects of the present disclosure provide engineered nucleic acid constructs that comprise a promoter operably linked to a nucleic acid that comprises (a) a nucleotide sequence encoding a single-stranded msr RNA, (b) a nucleotide sequence encoding a single-stranded msd DNA modified to contain a targeting sequence, and (c) a nucleotide sequence encoding a reverse transcriptase protein, wherein (a) and (b) are flanked by inverted repeat sequences. A promoter, in some embodiments, may be an inducible promoter. In some embodiments, the nucleotide sequence of (a) is upstream of the nucleotide sequence of (b), which is upstream of the nucleotide sequence of (c).

In some embodiments, a nucleic acid further comprises a nucleotide sequence that encodes a single-stranded DNA (ssDNA)-annealing recombinase protein. A ssDNA-annealing recombinase protein may be, for example, a Beta recombinase protein or a Beta recombinase protein homolog. In some embodiments, a ssDNA-annealing recombinase protein is a bacteriophage lambda Beta recombinase protein or a bacteriophage lambda Beta recombinase protein homolog. In some embodiments, a nucleotide sequence that encodes a ssDNA-annealing recombinase protein is downstream relative to the nucleotide sequence of (c).

Some aspects of the present disclosure provide cells that comprise at least one of the engineered nucleic acid constructs as provided herein. In some embodiments, a cell comprises at least two or at least three engineered nucleic acid constructs. In some embodiments, at least two of the promoters are different from each other.

Some aspects of the present disclosure provide cells that comprise (a) at least one of the engineered nucleic acid constructs as provided herein, and (b) a single-stranded DNA (ssDNA)-annealing recombinase protein. The ssDNA-annealing recombinase protein may be, for example, a Beta recombinase protein or a Beta recombinase protein homolog. In some embodiments, the cell comprises at least two or at least three engineered nucleic acid constructs. In some embodiments, at least two of the promoters are different from each other. In some embodiments, the cell comprises an engineered nucleic acid construct comprising a promoter operably linked to a nucleic acid encoding the ssDNA-annealing recombinase protein. The promoter may be, for example, an inducible promoter.

Also contemplated herein are cells that recombinantly expresses an Escherichia coli bacterial cell gene encoding XseA and/or XseB.

In some embodiments, cells of the present disclosure are Escherichia coli bacterial cells that contain a deletion of a gene encoding ExoI and/or RecJ. That is, in some embodiments, the bacterial cell does not express ExoI and/or RecJ.

Some aspects of the present disclosure provide methods that comprise delivering to cells at least one of the engineered nucleic acid constructs as provided herein, wherein the cell comprises a nucleotide sequence that is complementary to the targeting sequence. The nucleotide sequence that is complementary to the targeting sequence may be, for example, a genomic DNA sequence. Thus, in some embodiments, a targeting sequence recombines with a genomic DNA sequence.

Some aspects of the present disclosure provide methods that comprise delivering to cells (a) at least one of the engineered nucleic acid constructs as provided herein, and (b) an engineered nucleic acid construct comprising a promoter operably linked to a nucleic acid encoding a single-stranded DNA (ssDNA)-annealing recombinase protein, wherein the cell comprises a nucleotide sequence that is complementary to the targeting sequence. The ssDNA-annealing recombinase protein may be a Beta recombinase protein or a Beta recombinase protein homolog. The promoter operably linked to a nucleic acid encoding a ssDNA-annealing recombinase protein may be an inducible promoter. The nucleotide sequence that is complementary to the targeting sequence is, in some embodiments, a genomic DNA sequence. In some embodiments, at least two of the promoters are different from each other.

In some embodiments, methods further comprise exposing the cells to at least one signal that regulates transcription of at least one of the nucleic acids. In some embodiments, at least one signal activates transcription of at least one of the nucleic acids. In some embodiments, methods further comprise exposing the cells at least twice to at least one signal that regulates transcription of at least one of the nucleic acids. In some embodiments, methods further comprise exposing the cells at least twice over the course of at least 2 days to at least one signal that activates transcription of at least one of the nucleic acids.

In some embodiments, a signal is a chemical signal or a non-chemical signal. A non-chemical signal may be light, for example.

In some embodiments, a signal is an endogenous signal. Thus, the host cell may produce a signal that regulates (e.g., activates) transcription.

In some embodiments, methods further comprise calculating a recombination rate between the targeting sequence of the at least one engineered nucleic acid construct and a nucleotide sequence (e.g., genomic DNA sequence) complementary to the targeting sequence.

Some aspects of the present disclosure provide cells that comprise (a) a first engineered nucleic acid construct that comprises a first promoter operably linked to a first nucleic acid that comprises (i) a nucleotide sequence encoding a single-stranded msr RNA, and (ii) a nucleotide sequence encoding a single-stranded msd DNA modified to contain a targeting sequence, wherein (i) and (ii) are flanked by inverted repeat sequences, and (b) a second engineered nucleic acid construct that comprises a second promoter operably linked to a second nucleic acid that comprises a nucleotide sequence encoding a reverse transcriptase protein.

In some embodiments, the first and/or second promoter is an inducible promoter.

In some embodiments, the nucleotide sequence of (i) is upstream of the nucleotide sequence of (ii).

In some embodiments, the first or second nucleic acid further comprises a nucleotide sequence that encodes a single-stranded DNA (ssDNA)-annealing recombinase protein. The ssDNA-annealing recombinase protein may be a Beta recombinase protein or a Beta recombinase protein homolog. In some embodiments, the ssDNA-annealing recombinase protein is a bacteriophage lambda Beta recombinase protein or a bacteriophage lambda Beta recombinase protein homolog.

Some aspects of the present disclosure provide methods that comprise delivering to cells (a) a first engineered nucleic acid construct comprising a first inducible promoter operably linked to a first nucleic acid that comprises (i) a nucleotide sequence encoding a single-stranded msr RNA, (ii) a nucleotide sequence encoding a first single-stranded msd DNA modified to contain a first targeting sequence, and (iii) optionally a nucleotide sequence encoding a reverse transcriptase protein, wherein (i) and (ii) are flanked by inverted repeat sequences, and (b) a second engineered nucleic acid construct comprising a second inducible promoter operably linked to a second nucleic acid that comprises (iv) a nucleotide sequence encoding a single-stranded msr RNA, (v) a nucleotide sequence encoding a second single-stranded msd DNA modified to contain a second targeting sequence, and (vi) a optionally nucleotide sequence encoding a reverse transcriptase protein, wherein (iv) and (v) are flanked by inverted repeat sequences.

In some embodiments, the first and/or second nucleic acid (e.g., the first nucleic acid, the second nucleic acid, or both the first and second nucleic acids) comprises the nucleotide sequence encoding a reverse transcriptase protein. In some embodiments, the first and/or second nucleic acid does not comprises the nucleotide sequence encoding a reverse transcriptase protein, and the method further comprises delivering to the cells a third engineered nucleic acid construct comprising a promoter operably linked to a third nucleic acid that comprises a nucleotide sequence encoding a reverse transcriptase protein.

In some embodiments, the nucleotide sequence of (i) is upstream of the nucleotide sequence of (ii), which is upstream of the nucleotide sequence of (iii), and/or the nucleotide sequence of (iv) is upstream of the nucleotide sequence of (v), which is upstream of the nucleotide sequence of (vi).

In some embodiments, the method further comprises delivering to the cells an engineered nucleic acid construct that comprises a promoter operably linked to a nucleic acid encoding a single-stranded DNA (ssDNA)-annealing recombinase protein.

In some embodiments, the ssDNA-annealing recombinase protein is a Beta recombinase protein or a Beta recombinase protein homolog. In some embodiments, the first nucleic acid and/or the second nucleic acid further comprises a nucleotide sequence encoding a ssDNA-annealing recombinase protein. In some embodiments, the ssDNA-annealing recombinase protein is a Beta recombinase protein or a Beta recombinase protein homolog.

In some embodiments, the nucleotide sequence of (i) is upstream of the nucleotide sequence of (ii), which is upstream of the nucleotide sequence of (iii), which is upstream of the nucleotide sequence encoding a ssDNA-annealing recombinase protein and/or the nucleotide sequence of (iv) is upstream of the nucleotide sequence of (v), which is upstream of the nucleotide sequence of (vi), which is upstream of the nucleotide sequence encoding a ssDNA-annealing recombinase protein.

In some embodiments, the method further comprises exposing the cells to a first signal that regulates transcription of the first nucleic acid and a second signal that regulates transcription of the second nucleic acid.

In some embodiments, the cells are exposed to the first signal under conditions that permit recombination of the first targeting sequence of the first single-stranded msd DNA and a nucleotide sequence complementary to the first targeting sequence, and then the cells are exposed to the second signal under conditions that permit recombination of the second targeting sequence of the second single-stranded msd DNA and a nucleotide sequence complementary to the second targeting sequence.

In some embodiments, the exposing step is repeated at least once. In some embodiments, the exposing step is repeated at least once over the course of at least 2 days.

In some embodiments, the first signal and/or the second signal is a chemical signal or a non-chemical signal. In some embodiments, the first signal and/or second signal is a non-chemical signal, and the non-chemical signal is light.

In some embodiments, the first signal and/or second signal is an endogenous signal.

In some embodiments, the first targeting sequence is complementary to a nucleotide sequence located in the genome of the cell, and the second targeting sequence is complementary to the first targeting sequence. A “genomic sequence” and a “sequence located in the genome of a cell” are used interchangeably herein.

In some embodiments, the first targeting sequence is complementary to a nucleotide sequence located in the genome of the cell, and the second targeting sequence is complementary to a nucleotide sequence located in the genome of the cell.

In some embodiments, the first targeting sequence is different from the second targeting nucleotide sequence.

In some embodiments, the methods further comprise calculating a recombination rate between the first targeting sequence and a nucleotide sequence complementary to the first targeting sequence and/or calculating a recombination rate between the second targeting sequence and a nucleotide sequence complementary to the second targeting sequence.

Some aspects of the present disclosure provide cells that comprise (a) a first engineered nucleic acid construct comprising a first inducible promoter operably linked to a first nucleic acid encoding a reporter protein containing at least one genetic element that prevents transcription of the reporter protein, and (b) a second engineered nucleic acid construct comprising a second inducible promoter operably linked to a second nucleic acid that comprises (i) a nucleotide sequence encoding a single-stranded msr RNA, (ii) a nucleotide sequence encoding a single-stranded msd DNA modified to contain a targeting sequence complementary to the at least one genetic element that prevents transcription of the reporter protein, and (iii) optionally a nucleotide sequence encoding a reverse transcriptase protein, wherein (i) and (ii) are flanked by inverted repeat sequences. In some embodiments, the nucleotide sequence of (i) is upstream of the nucleotide sequence of (ii), which is upstream of the nucleotide sequence of (iii).

In some embodiments, the cell further comprises an engineered nucleic acid construct that comprises a promoter operably linked to a nucleic acid encoding a Beta recombinase protein or a Beta recombinase protein homolog.

In some embodiments, the second nucleic acid further comprises a nucleotide sequence encoding a single-stranded DNA (ssDNA)-annealing recombinase protein. For example, the ssDNA-annealing recombinase protein may be a Beta recombinase protein or a Beta recombinase protein homolog.

In some embodiments, the nucleotide sequence of (i) is upstream of the nucleotide sequence of (ii), which is upstream of the nucleotide sequence of (iii), which is upstream of the nucleotide sequence encoding a ssDNA-annealing recombinase protein.

In some embodiments, the at least one genetic element is at least one stop codon.

In some embodiments, the first engineered nucleic acid construct is located genomically.

Some aspects of the present disclosure provide methods that comprise (a) providing cells that comprise a first engineered nucleic acid construct comprising a first inducible promoter operably linked to a first nucleic acid encoding a reporter protein containing at least one genetic element that prevents transcription of the reporter protein, and (b) delivering to the cells a second engineered nucleic acid construct comprising a second inducible promoter operably linked to a second nucleic acid that comprises (i) a nucleotide sequence encoding a single-stranded msr RNA, (ii) a nucleotide sequence encoding a single-stranded msd DNA modified to contain a targeting sequence complementary to the at least one genetic element that prevents transcription of the reporter protein, and (iii) optionally a nucleotide sequence encoding a reverse transcriptase protein, wherein (i) and (ii) are flanked by inverted repeat sequences. In some embodiments, the nucleotide sequence of (i) is upstream of the nucleotide sequence of (ii), which is upstream of nucleotide sequence of the nucleotide sequence of (iii).

In some embodiments, the method further comprises delivering to the cells an engineered nucleic acid construct that comprises a promoter operably linked to a nucleic acid encoding a single-stranded DNA (ssDNA)-annealing recombinase protein. In some embodiments, the second nucleic acid further comprises a nucleotide sequence encoding a ssDNA-annealing recombinase protein. In some embodiments, the ssDNA-annealing recombinase protein is a Beta recombinase protein or a Beta recombinase protein homolog.

In some embodiments, the nucleotide sequence of (i) is upstream of the nucleotide sequence of (ii), which is upstream of the nucleotide sequence of (iii), which is upstream of the nucleotide sequence encoding a ssDNA-annealing recombinase protein.

In some embodiments, the methods further comprise exposing the cells to a first signal that regulates transcription of the first nucleic acid and a second signal that regulates transcription of the second nucleic acid. In some embodiments, the cells are exposed to the second signal under conditions that permit transcription of the second nucleic acid and recombination of the targeting sequence, and then the cells are exposed to the first signal under conditions that permit transcription of the first nucleic acid. In some embodiments, the cells are exposed to the second signal under conditions that permit transcription of the second nucleic acid and recombination of the targeting sequence, exposure of the cells to the second signal is discontinued, and then the cells are exposed to the first signal under conditions that permit transcription of the first nucleic acid.

In some embodiments, the methods further comprise calculating a recombination rate between the targeting sequence and the at least one genetic element.

In some embodiments, the at least one genetic element is at least one stop codon.

In some embodiments, the first engineered nucleic acid construct is located genomically.

Some aspects of the present disclosure provide cells that comprise (a) a first engineered nucleic acid construct comprising a first inducible promoter operably linked to a first nucleic acid encoding a reporter protein containing at least one genetic element that prevents translation of the reporter protein, (b) a second engineered nucleic acid construct comprising a second inducible promoter operably linked to a second nucleic acid that comprises (i) a nucleotide sequence encoding a single-stranded msr RNA, (ii) a nucleotide sequence encoding a single-stranded msd DNA modified to contain a targeting sequence that is complementary to the at least one genetic element that prevents translation of the reporter protein, and (iii) optionally a nucleotide sequence encoding a reverse transcriptase protein, wherein (i) and (ii) are flanked by inverted repeat sequences, and (c) a third engineered nucleic acid construct comprising a third inducible promoter operably linked to a third nucleic acid encoding a single-stranded DNA (ssDNA)-annealing recombinase protein. In some embodiments, the ssDNA-annealing recombinase protein is a Beta recombinase protein or a Beta recombinase protein homolog. In some embodiments, the at least one genetic element is at least one stop codon. In some embodiments, the first engineered nucleic acid construct is located genomically. In some embodiments, the nucleotide sequence of (i) is upstream of the nucleotide sequence of (ii), which is upstream of the nucleotide sequence of (iii).

Some aspects of the present disclosure provide methods that comprise (a) providing cells that comprise a first engineered nucleic acid construct comprising a first inducible promoter operably linked to a first nucleic acid encoding a reporter protein containing at least one genetic element that prevents translation of the reporter protein, and (b) delivering to the cells a second engineered nucleic acid construct comprising a second inducible promoter operably linked to a second nucleic acid that comprises (i) a nucleotide sequence encoding a single-stranded msr RNA, (ii) a nucleotide sequence encoding a single-stranded msd DNA modified to contain a targeting sequence that is complementary to the at least one genetic element that prevents translation of the reporter protein, and (iii) optionally a nucleotide sequence encoding a reverse transcriptase protein, wherein (i) and (ii) are flanked by inverted repeat sequences.

In some embodiments, the methods further comprise delivering to the cells a third engineered nucleic acid construct comprising a third inducible promoter operably linked to a third nucleic acid encoding a single-stranded DNA (ssDNA)-annealing recombinase protein.

In some embodiments, the ssDNA-annealing recombinase protein is a Beta recombinase protein or a Beta recombinase protein homolog.

In some embodiments, the methods further comprise exposing the cells to a first signal that regulates transcription of the first nucleic acid, a second signal that regulates transcription of the second nucleic acid, and a third signal that regulates transcription of the third nucleic acid. In some embodiments, the cells are exposed to the second and third signal under conditions that permit transcription of the second and third nucleic acids, respectively, and recombination of the targeting sequence, and then the cells are exposed to the first signal under conditions that permit transcription of the first nucleic acid.

In some embodiments, the methods further comprise calculating a recombination rate between the targeting sequence and the at least one genetic element.

In some embodiments, the at least one genetic element is at least one stop codon.

In some embodiments, the first engineered nucleic acid construct is located genomically.

Some aspects of the present disclosure provide methods of performing multiplex automated genome editing, comprising (a) delivering to cells having a genome at least one of the engineered nucleic acid constructs as provided herein, and (b) culturing the cells under conditions suitable for nucleic acid expression and integration of the single-stranded msd DNA into the genome of cells of (a).

Some aspects of the present disclosure provide methods of producing a nucleic acid nanostructure, comprising (a) delivering to cells a plurality of the engineered nucleic acid constructs as provided herein, wherein single-stranded msd DNAs are designed to self-assemble through complementary nucleotide base-pairing into a nucleic acid nanostructure; and (b) culturing the cells under conditions suitable for nucleic acid expression and self-assembly. Conditions suitable for nucleic acid self-assembly include conditions that permit annealing of complementary (e.g., fully complementary) nucleic acids. In some embodiments, the nucleic acid nanostructure is a two-dimensional or a three-dimensional nucleic acid nanostructure. In some embodiments, the nucleic acid nanostructure is a nucleic acid nanorobot.

### BRIEF DESCRIPTION OF THE DRAWINGS

FIGS. 1A-1C illustrate that SCRIBE (Synthetic Cellular Recorders Integrating Biological Events) enables in vivo DNA writing and read/write memory registers that can be used to record analog memory in the collective genomic DNA of live cell populations. FIG. 1A shows a schematic of a writing phase (SEQ ID NO: 32 (left), SEQ ID NO: 33 (right)). FIG. 1B shows a schematic of an induction/recording phase. FIG. 1C shows a schematic of integrated write and read phases (SEQ ID NO: 34 (top), SEQ ID NO: 35 (bottom)).

FIGS. 2A-2G illustrate that SCRIBE uses bacterial retrons to generate ssDNAs that are incorporated into genomic target loci when expressed in concert with the Beta protein, thus enabling the magnitude of inputs to be recorded in the genomic DNA of bacterial populations. The sequences in FIG. 2D correspond to SEQ ID NO: 36 (top) and SEQ ID NO: 37 (bottom).

FIGS. 3A-3G illustrate that SCRIBE can write multiple different DNA mutations into a common target loci or multiple DNA mutations into independent target loci for multiplexed in vivo memories.

FIGS. 4A and 4B illustrate simultaneous writing into two genomic loci within individual cells.

FIGS. 5A-5F illustrate optogenetic genome editing and analog memory for long-term recording of input signal exposure times in the genomic DNA of live cell populations.

FIG. 6 illustrates the recombination rate for the SCRIBE circuit (shown in FIG. 2C) when the system is induced with both isopropyl β-D-1-thiogalactopyranoside (IPTG) (1 mM) and aTc (100 ng/ml). The recombination rate was estimated by calculating the slope of the regression line for the data shown in FIG. 5F (induction pattern II) and multiplying that slope by a factor of two as described in the deterministic model

$$( r = 2 \frac{df}{dt} = 2 * 7.7 * {10}^{- 5} = 1.54 * {10}^{- 4} ) .$$

In FIG. 5F, the cultures were diluted 1:1000 at the beginning of each day and grown to saturation by the end of the day. Thus, the x-axis in FIG. 5F corresponds to log₂(1000)≈10 generations per day.

FIGS. 7A-7C illustrate a deterministic model and stochastic simulation describing the long-term recording of information into genomically encoded memory with the SCRIBE system at three different recombination rates. FIG. 7A: r=10⁻⁹; FIG. 7B: r=0.00015, and FIG. 7C: r=0.005. At a very low recombination rate (e.g., r=10⁻⁹), the model predicts a linear increase in the frequency of recombinants in the population. However, the simulation shows no steady increase in the recombinant frequency, likely because the sampling of cells after every 10 generations to start a fresh culture in the simulation does not carry over a representative number of recombinant cells. At very high recombination rates (e.g., r=0.005), both the model and simulation initially show a linear increase in the recombination frequencies but this trend quickly starts to saturate. At a moderate recombination rate (e.g., r=0.00015), both the model and simulation show a linear increase in the recombinant frequencies over hundreds of generations. This linear trend starts to saturate as the recombinant frequency in the population approaches 5% (not shown).

FIGS. 8A-8F illustrate SCRIBE memory operations that can be decoupled into independent Input, Write, and Read operations, thus facilitating greater control over addressable memory registers in genomic tape recorders and the creation of sample-and-hold circuits.

FIGS. 9A and 9B illustrate the effect of host factors on the recombination efficiency of the SCRIBE system. The constructs shown in FIG. 2C were transformed to E. coli cells with genetic backgrounds shown in the x-axis (wild type (WT) refers to DH5alpha PRO GalK::KanR). The recombination efficiency was calculated as described for FIG. 2C. FIG. 9B illustrates a proposed model describing the source of recombinogenic oligonucleotides suggested based on recombination efficiency in different knockout strains. Only short msDNA molecules are recombinogenic. The long msDNA molecules are first processed by XseA (ExoVII) (or some cellular endonucleases) to produce smaller ssDNA pieces. The small ssDNA molecules that are produced can be recombined into target locus via beta-mediated recombination. The small ssDNA molecules however can be further processed into single nucleotides (that are not non-recombinogenic) by RecJ and ExoI exonucleases.

FIG. 10 illustrates that the efficiency of recombination in a DH5alpha recJΔ XonAΔ background is increased over time in cells expressing the SCRIBE(KanR)_{ON} cassette and GFP (which was used as a passive control). The recombination efficiency in DH5alpha recJΔ XonAΔ background can be further enhanced by overexpression of ExoVII complex (XseA and XseB).

### DETAILED DESCRIPTION OF THE INVENTION

Deoxyribonucleic acid (DNA) is the media for the storage and transmission of information in living cells. Due to its high storage capacity, durability, ease of duplication, and high-fidelity maintenance of information, DNA as an artificial storage media has garnered much interest. Recent technological advances have made it possible to read and write information in DNA in vitro and even rewrite information encoded in entire chromosomes or incorporate unnatural genetic alphabets. However, existing technologies for in vivo autonomous recording of information in cellular memory (e.g., genetically) are limited in their storage capacity and scalability.

Epigenetic memory devices such as bistable toggle switches and positive-feedback loops require orthogonal transcription factors and can lose their digital state due to environmental fluctuations or cell death. Recombinase-based devices enable the writing and storage of digital information in the DNA of living cells, where binary bits of information are stored in the orientation of large stretches of DNA; however, these devices do not efficiently exploit the full capacity of DNA for information storage. Recording a single bit of information with these devices often requires at least a few hundred base-pairs of DNA, overexpression of a recombinase protein to invert the target DNA, and engineering recombinase-recognition sites into target loci in advance. The scalability of this type of memory is further limited by the number of orthogonal recombinases that can be used in a single cell. Finally, epigenetic and recombinase-based memory devices store digital information, and their recording capacity is exhausted within a few hours of induction. Thus, the use of these devices has been restricted to recording the digital presence or absence of inputs and they have not been adapted to record analog information, such as the magnitude and the time course of inputs over extended periods of time (e.g., multiple days or more).

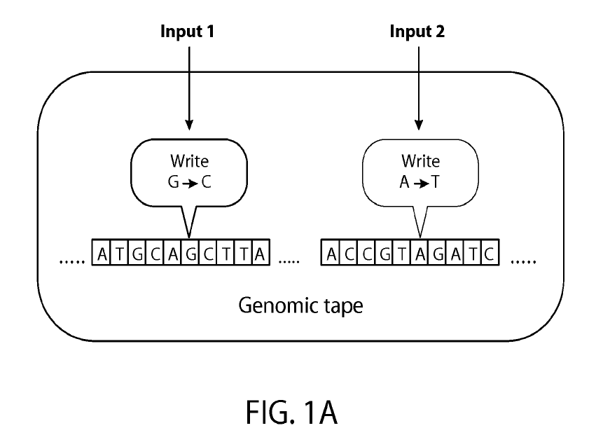

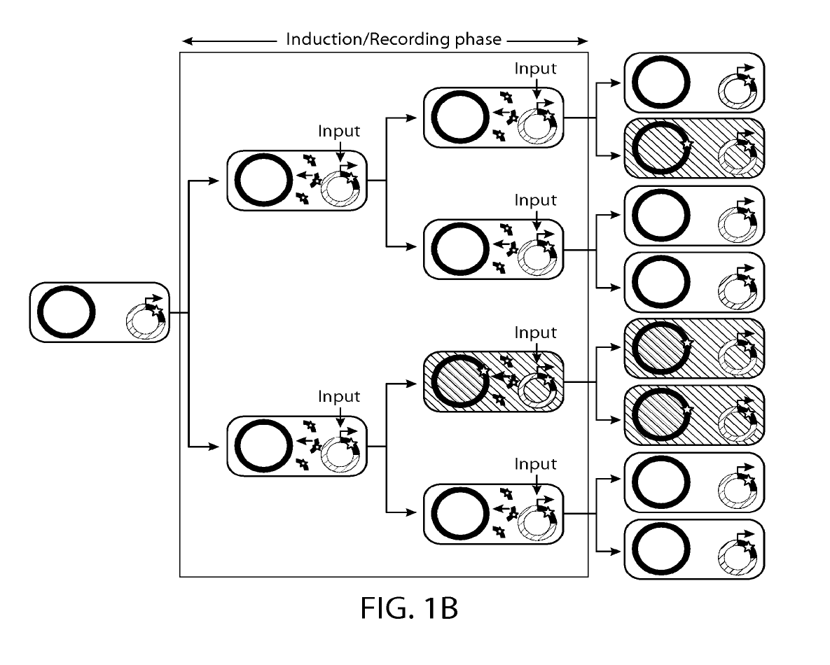

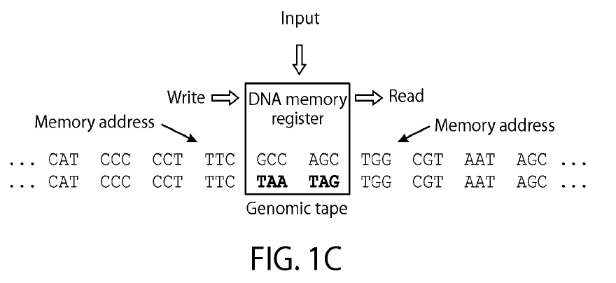

Provided herein, in some aspects, are platforms for in vivo DNA writing that use the genomes of live organisms to store information (FIG. 1A). This platform is referred to herein as SCRIBE (Synthetic Cellular Recorders Integrating Biological Events). A compact, modular memory device was developed to generate single-stranded DNA (ssDNA) inside live cells in response to a range of regulatory signals, such as, for example, small chemical inducers and light. These ssDNAs uniquely address specific target loci based on sequence homology and introduce precise mutations into genomic DNA (FIG. 1B). The memory device can be easily reprogrammed by changing the ssDNA template. Genomically-stored information can be read out using a suite of flexible techniques, including, for example, reporter genes, functional assays and DNA sequencing (e.g., high-throughput sequencing). SCRIBE memory does not just record the absence or presence of arbitrary inputs (digital signals represented as binary ‘0s’ or ‘1s’), as in previously described recombinase-based or epigenetic memories that focus on memory state within single cells. Instead, by encoding information into the collective genomic DNA of cell populations, SCRIBE can, in some embodiments, track the magnitude and long-term temporal behavior of inputs, which are considered “analog signals” because they can vary over a wide range of continuous values. This analog memory, in some embodiments, leverages the large number of cells in bacterial cultures for distributed information storage and archives event histories in the fraction of cells in a population that carry specific mutations (FIG. 1B).

The present disclosure demonstrates that SCRIBE can be multiplexed, for example, to record multiple inputs and that SCRIBE-induced mutations can be written and erased. Further, the present disclosure shows that “Input,” “Write” and “Read” operations can be decoupled, for example, for genomically-encoded memories, thus enabling the creation of genetic “sample-and-hold” circuits, the integration of logic and analog memory, and the use of small stretches of genomic DNA “tape” as addressable read/write memory registers (FIG. 1C).

In some embodiments, methods and compositions of the present disclosure enable in vivo DNA writing and read/write memory registers that can be used to record analog memory in the collective genomic DNA of live cell populations. FIG. 1A shows that the genomes of live cells can be used as tape recorders for storing information on multiple inputs in the form of long-lasting genetic modifications within DNA memory registers. FIG. 1B shows that in the presence of an input, such as a chemical inducer or light, short single-stranded DNA (ssDNA) molecules (dark gray curved lines) are produced inside the cells from a plasmid-borne cassette (light gray circles). These ssDNAs uniquely address specific target loci in the genome (dark gray circles) as defined by sequence homologies. These ssDNAs are integrated into the genome, a process that is facilitated by a concomitantly expressed ssDNA-specific recombinase, thus resulting in the de novo introduction of precise mutations (stars) into the genome. The frequency of cells in the population that carry specific targeted mutations (shaded cells) accumulates as a function of the magnitude and duration of the input, thus enabling analog memory to be stored in the form of allele frequencies in the population. FIG. 1C shows that genomic DNA can be used as addressable read/write memory registers, where “Input”, “Write” and “Read” operations can be independently controlled, and memory addressing is programmable based on sequence homologies. Intracellularly expressed ssDNAs (top strand, medium gray) are addressed to target genomic loci (bottom strand, light gray), where they recombine into the target site and introduce precise modifications. Up to 4⁶=4096 unique information-encoding sequences can be potentially stored in a 6-bp stretch of DNA.

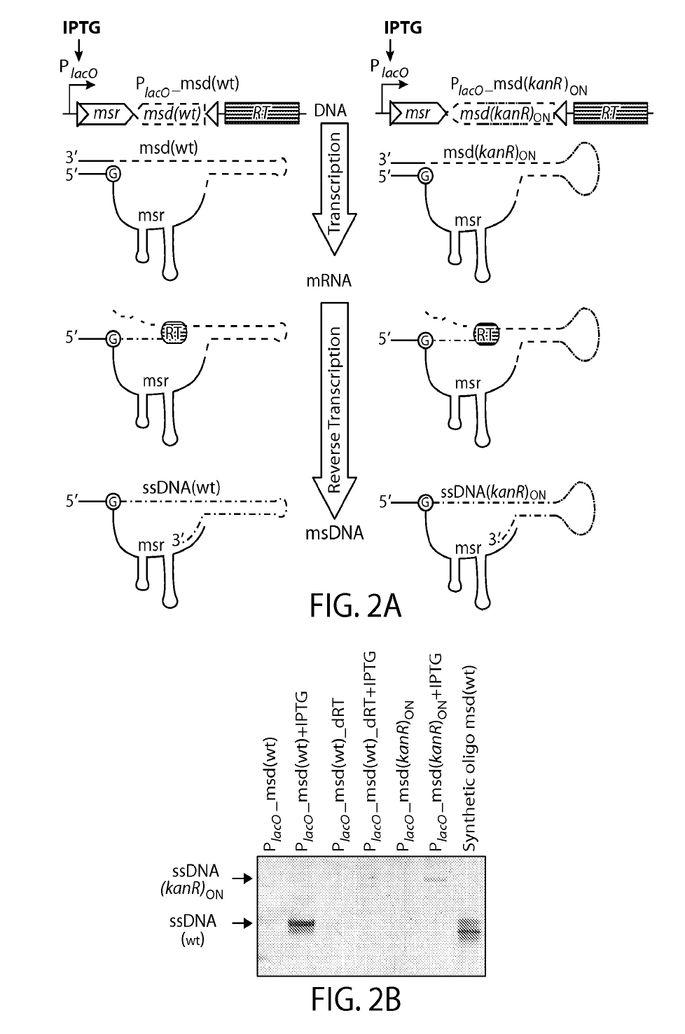

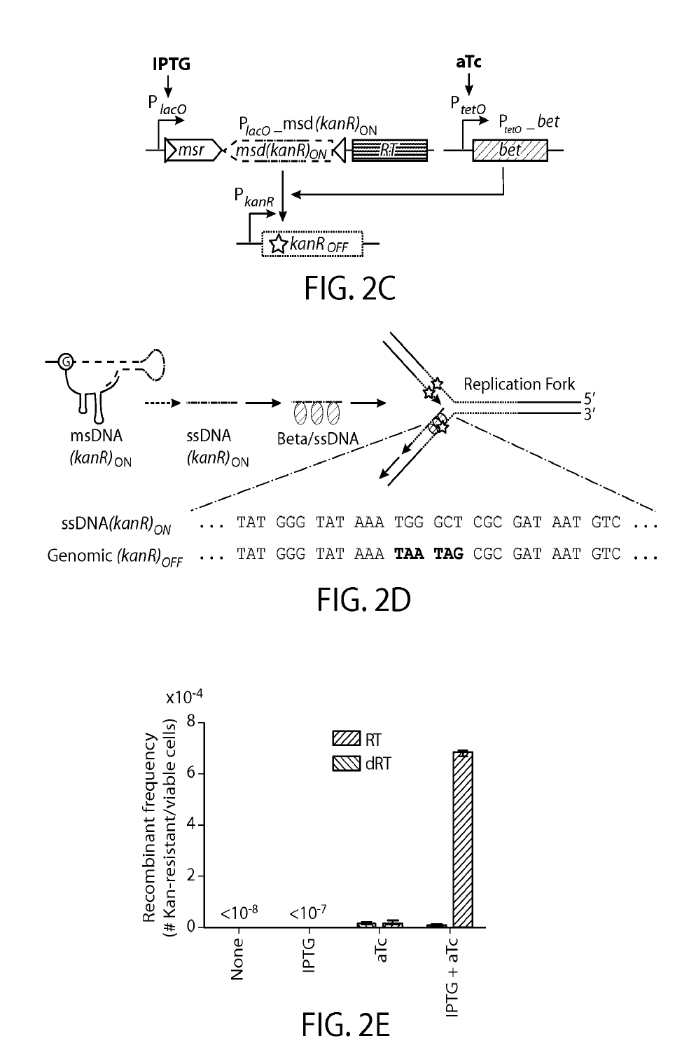

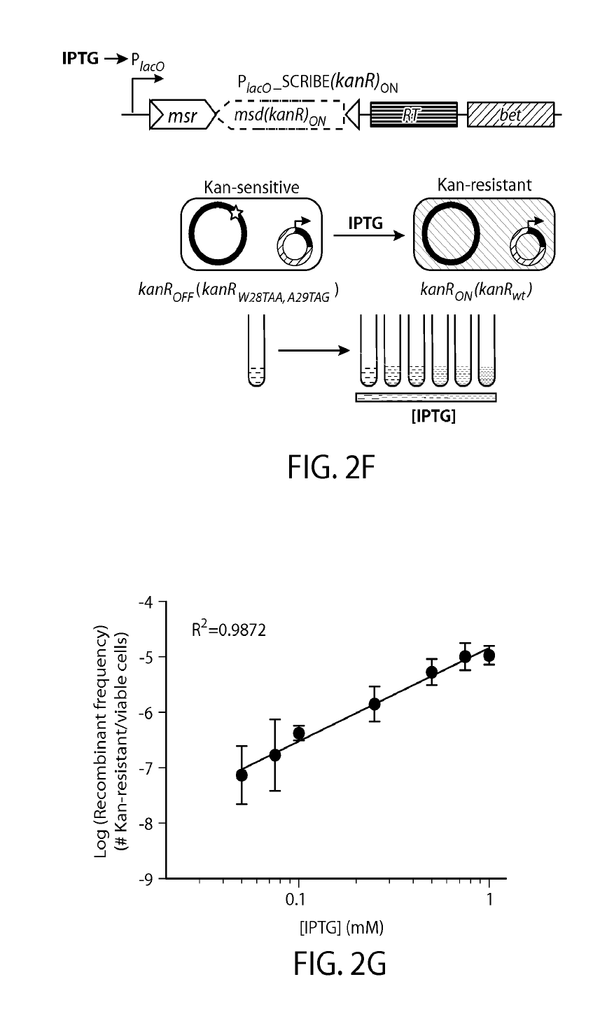

In some embodiments, methods and compositions of the present disclosure can be used with bacterial retrons to generate ssDNAs that are incorporated into genomic target loci when expressed in concert with Beta protein, thus enabling the magnitude of inputs to be recorded in the genomic DNA of bacterial populations. FIG. 2A shows an example of a molecular mechanism of ssDNA generation inside of live cells by retrons. The wild-type retron cassette from E. coli BL21 is placed under the control of an IPTG-inducible promoter (P_{lacO}) in E. coli DH5αPRO cells. FIG. 2B shows a denaturing gel visualization of retron-mediated ssDNAs produced in live bacteria. Overnight cultures harboring IPTG-inducible plasmids expressing msd(wt), msd(wt) with deactivated reverse transcriptase (RT) (msd(wt)_dRT), or msd(kanR)_{ON} were grown overnight with or without IPTG (1 mM). Total RNA was purified from these samples and treated with RNase A to remove RNA species and the msr moiety. These samples were then resolved on a 10% denaturing gel and visualized with SYBR-Gold. A synthetic oligonucleotide with the same sequence as the ssDNA(wt) was used as a molecular size marker. FIGS. 2D and 2C show a kanR reversion assay that can be to measure the efficiency of in vivo DNA writing. Reporter cells contain a genomic kanR cassette that is deactivated by two premature stop codons inside the open reading frame (ORF) (kanR_{OFF}). A ssDNA containing the wild-type kanR sequence (ssDNA(kanR)_{ON}) is expressed from a plasmid when induced by IPTG. The ssDNA(kanR)_{ON} is addressed to target the homologous kanR_{OFF} loci on the genome, a process that is facilitated by the co-expression of Beta recombinase (bet), which is induced by anhydrotetracycline (aTc). FIG. 2E shows a graph of data obtained from the following experiment. Overnight cultures of the kanR_{OFF} strain containing the IPTG-inducible msd(kanR)_{ON} cassette and the aTc-inducible bet gene were diluted (1:1000) and then grown in the presence or absence of IPTG (1 mM) and aTc (100 ng/ml) for 24 hours. Induction of the cells with both aTc and IPTG led to a ˜10⁵-fold increase in the number of kanamycin (Kan)-resistant cells in the population compared to the non-induced cells. This effect was largely abolished when the reverse transcriptase (RT) was deactivated, indicating that in vivo genome writing depends on RT activity and ssDNA production. FIG. 2F shows that SCRIBE enables analog memory that records the magnitude of inputs in the genomic DNA of a cell population. The msd(kanR)_{ON} cassette and bet were combined into a synthetic operon (referred to as SCRIBE(kanR)_{ON}) and placed under the control of an IPTG-inducible promoter. Overnight cultures of kanR_{OFF} reporter cells harboring P_{lacO}_{_}SCRIBE(kanR)_{ON} were diluted into fresh media with different concentrations of IPTG and then grown for 24 hours at 30° C. FIG. 2G shows a graph of data obtained from the following experiment. The number of Kan-resistant cells in a population containing the circuit shown in FIG. 2F increased linearly (on log-log scale) as the concentration of IPTG increased, indicating that SCRIBE can encode analog memory that records the magnitude of an input into genomic DNA (error bars indicate the standard error of the mean for three independent biological replicates).

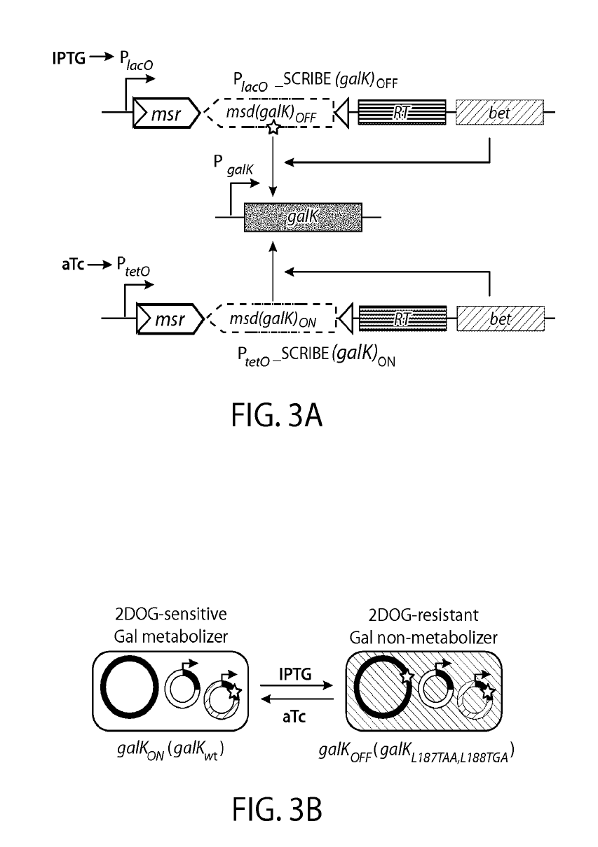

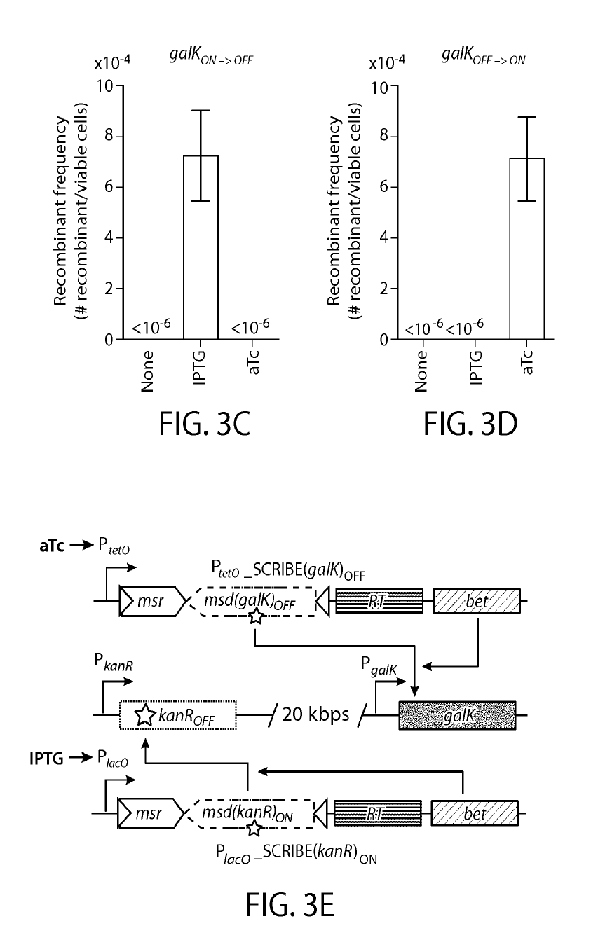

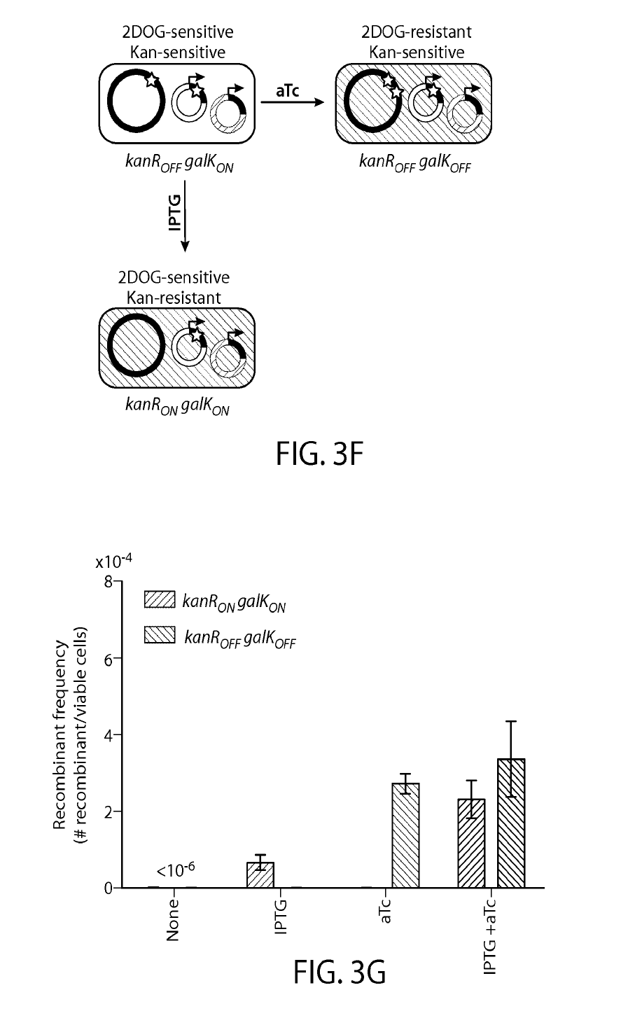

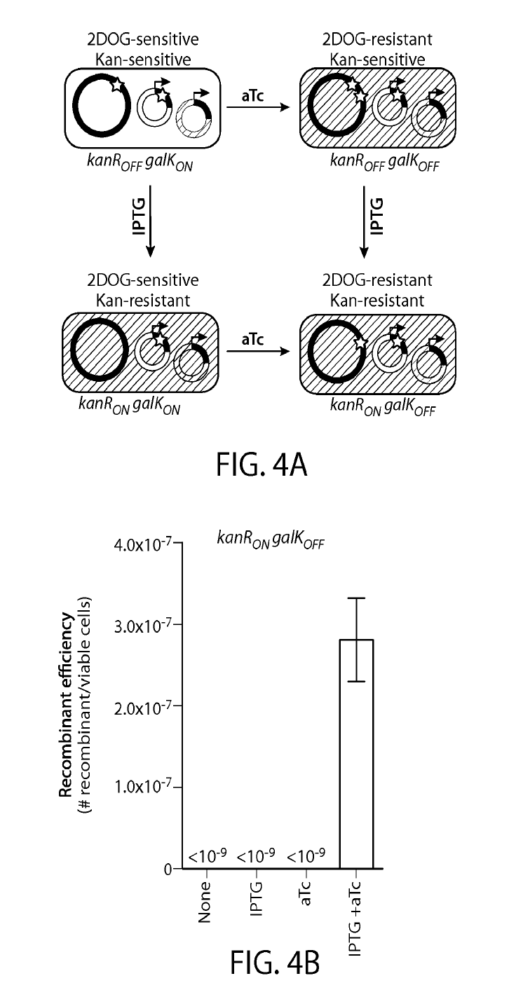

In some embodiments, methods and compositions of the present disclosure can be used to write multiple different DNA mutations into common target loci or multiple DNA mutations into independent target loci for multiplexed in vivo memories. FIG. 3A shows the creation of a complementary set of SCRIBE cassettes to write and erase (rewrite) information in the genomic galK locus using two different chemical inducers. Induction of the cells with IPTG induces expression of the SCRIBE(galK)_{OFF} cassette, which introduces two stop codons into the galK gene. These premature stop codons can be reverted back to the wild-type sequence by a second ssDNA expressed from an aTc-inducible SCRIBE(galK)_{ON} cassette. FIG. 3B shows that IPTG induces the conversion of galK_{ON} to galK_{OFF}, whereas aTc induces the conversion of galK_{OFF} to galK_{ON}. galK is a selectable/counterselectable marker that enables the frequency of the galK_{ON} and galK_{OFF} alleles in the population to be determined by plating the cells on either galactose or glycerol+2DOG plates, respectively. FIG. 3C shows a graph of data obtained from the following experiment. galK_{ON} cells harboring the circuits shown in FIG. 3C were induced with either IPTG (1 mM) or aTc (100 ng/ml) for 24 hours and the allele frequencies in the population were determined by plating the cells on appropriate selective conditions. Only cultures induced with IPTG produced significant number of cells with the galK_{OFF} allele. FIG. 3D shows a graph of data obtained from the following experiment. galK_{OFF} cells (obtained from the experiment described in FIG. 3C)) were induced with IPTG (1 mM) or aTc (100 ng/ml) for 24 hours and the allele frequencies in the population were determined by plating the cells on appropriate selective conditions. Only cultures induced with aTc produced significant number of cells with galK_{ON} alleles. FIG. 3E shows that SCRIBE enables multiplexed analog memories that can record multiple inputs into different genomic loci. This was demonstrated by targeting genomic kanR_{OFF} and galK_{ON} loci with IPTG-inducible and aTc-inducible SCRIBE cassettes, respectively. FIG. 3F shows induction of kanR_{OFF} galK_{ON} cells with IPTG or aTc generates cells with the kanR_{ON} galK_{ON} or kanR_{OFF} galK_{OFF} genotypes, respectively. FIG. 3G shows kanR_{OFF} galK_{ON} reporter cells containing the circuits in FIG. 3E induced with different combinations of IPTG (1 mM) and aTc (100 ng/ml) for 24 h at 30° C., and the fraction of cells with the various genotypes were determined by plating the cells on appropriate selective media. Induction with IPTG led to the production of kanR_{ON} galK_{ON} cells in the population. Induction with aTc led to the production of kanR_{OFF} galK_{OFF} cells in the population. Induction with both aTc and IPTG led to the production of both kanR_{ON} galK_{ON} and kanR_{OFF} galK_{OFF} cells in the population. Very few single cells in samples induced with both aTc and IPTG were converted to kanR_{ON} galK_{OFF} (FIG. 4B; error bars indicate the standard error of the mean for three independent biological replicates).

In some embodiments, methods and compositions of the present disclosure can be used to simultaneous write into two genomic loci within individual cells. FIG. 4A shows kanR_{OFF} galK_{ON} reporter cells harboring aTc-inducible SCRIBE(galK)ₒⱼ and IPTG-inducible SCRIBE(kanR)_{ON} (as shown in FIG. 3E-G) were induced with both IPTG (1 mM) and aTc (100 ng/ml). FIG. 4B shows a graph illustrating that under combined aTc and IPTG induction, very few single cells were converted to kanR_{ON} galK_{OFF}, compared with the frequencies of kanR_{OFF} galK_{OFF} and kanR_{ON} galK_{ON} cells shown in FIG. 3G. No kanR_{ON} galK_{OFF} cells were detected in samples induced with either aTc or IPTG alone or non-induced cells (error bars indicate the standard error of the mean for three independent biological replicates).

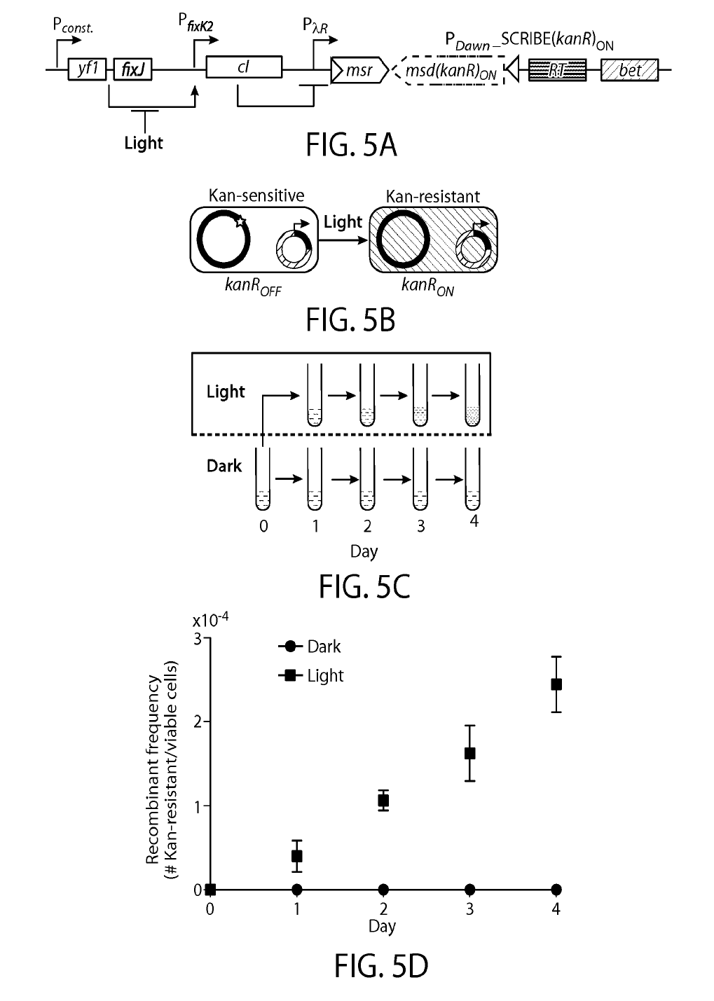

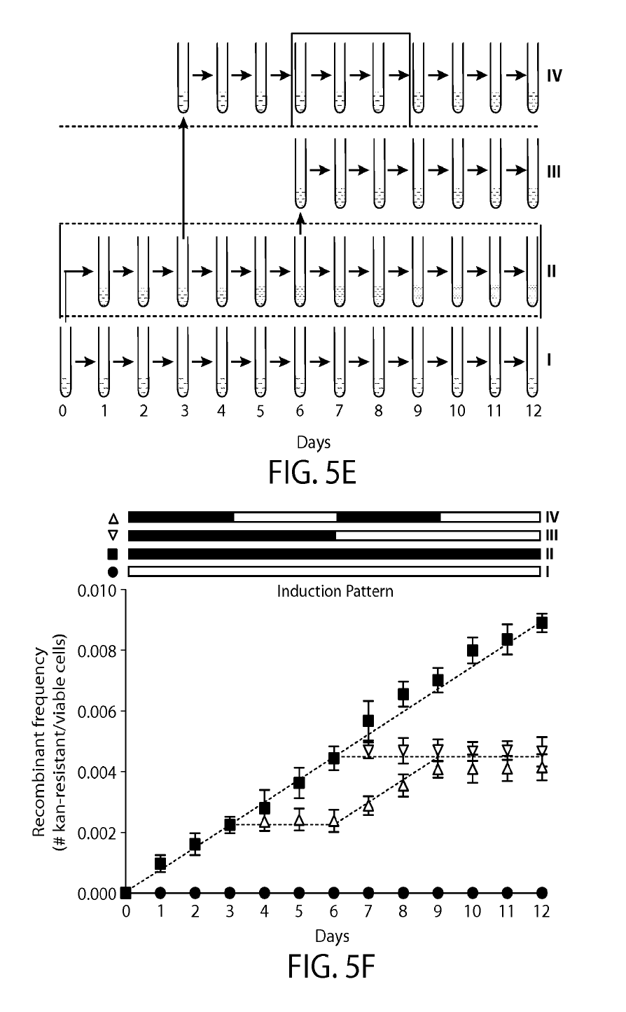

In some embodiments, methods and compositions of the present disclosure can be used for optogenetic genome editing and analog memory for long-term recording of input signal exposure times in the genomic DNA of live cell populations. FIG. 5A shows expression of the SCRIBE(kanR)_{ON} coupled to an optogenetic system (P_{Dawn}). The yfl/fixJ synthetic operon was expressed from a constitutive promoter—its products cooperatively activate the P_{fixK2} promoter, which drives lambda repressor (cI) expression, which subsequently represses the SCRIBE(kanR)_{ON} cassette. Light inhibits the interaction between yfl and fixJ, leading to the generation of ssDNA(kanR)_{ON} and Beta expression. FIG. 5B shows that exposure of cells to light converts kanR_{OFF} to kanR_{ON}. FIG. 5C shows that cells harboring the circuit in FIG. 5A were grown overnight at 37° C. in the dark, diluted 1:1000, and then incubated for 24 h at 30° C. in the dark (no shading) or in the presence of light (yellow shading). Subsequently, cells were diluted by 1:1000 and grown for another 24 h at 30° C. in the dark or in the presence of light. The dilution/regrowth cycle was performed for four consecutive days. FIG. 5D shows a graph of kanR allele frequencies in populations that were determined by sampling the cultures after each 24-hour period. The fraction of Kan-resistant colonies increased linearly with the amount of time the cultures were exposed to light (squares). No Kan-resistant colonies were detected in the cultures grown in the dark (circles). FIG. 5E shows that SCRIBE analog memory records the total time exposure to a given input, regardless of the underlying induction pattern. Cells harboring the circuit shown in FIG. 2C were grown in four different patterns (I-IV) over a twelve-day period, where induction by IPTG (1 mM) and aTc (100 ng/mL) is represented by dark gray shading. At the end of each 24 h incubation period, cells were diluted by 1:1000 into fresh media. The number of Kan-resistant cells in the cultures was determined at the end of each day. FIG. 5F shows a graph illustrating that non-induced cell populations (pattern I, black circles) showed minimal numbers of Kan-resistant cells. Cell populations induced continuously during the twelve-day period (pattern II, squares) exhibited a linear increase in the frequency of Kan-resistant cells. Cell populations that were induced for a total of six days (pattern III, upside-down triangles and pattern IV, upright triangles) had similar frequencies of Kan-resistant cells by the end of the experiment, even though they had different temporal induction patterns. Further, cell populations exposed to pattern III and pattern IV maintained their analog memory state, represented in the frequency of Kan-resistant cells in the population, during non-induced periods, thus demonstrating stable recording of genomic memory over long periods of time. Dashed lines represent the recombinant allele frequencies predicted by the model (see Examples). Error bars indicate the standard error of the mean for three independent biological replicates.

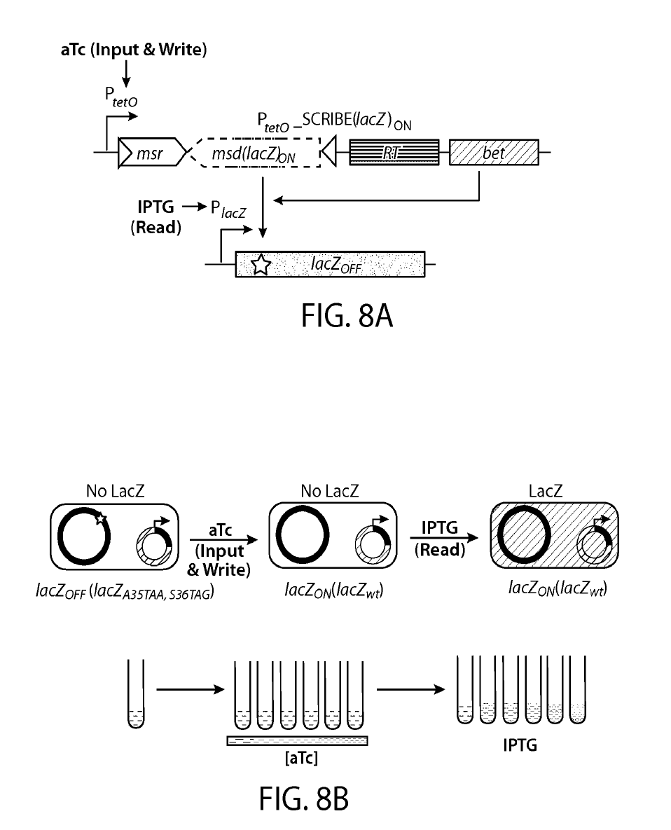

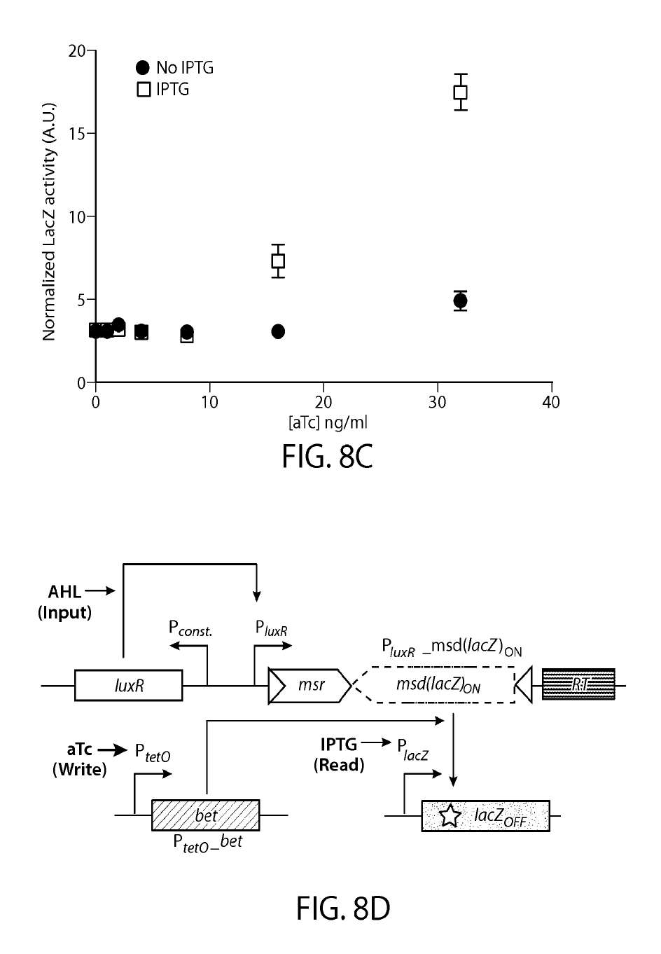

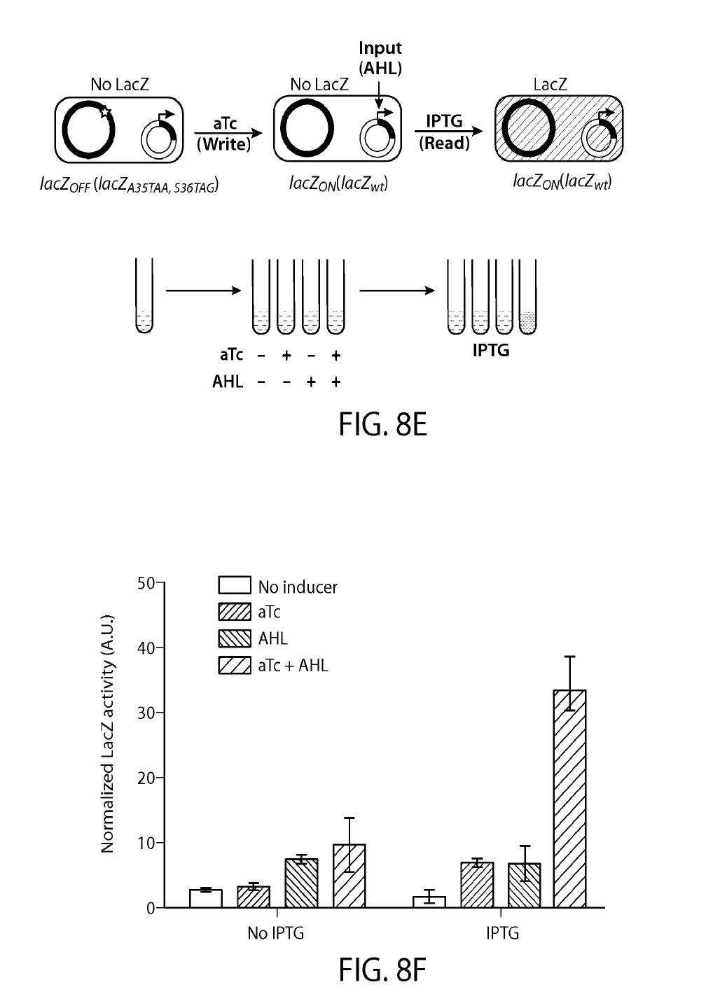

In some embodiments, methods and composition of the present disclosure can be used to build a circuit where a chemical inducer (e.g., aTc) serves as the “Input & Write” signal and IPTG triggers a “Read” operation. For example, as shown in FIG. 8A, an IPTG-inducible lacZ_{OFF} locus was created in the DH5αPRO background, which contains the full-length lacZ gene with two premature stop codons inside the open-reading frame. Expression of ssDNA(lacZ)_{ON} from the aTc-inducible SCRIBE(lacZ)_{ON} cassette results in the reversion of the stop codons inside lacZ_{OFF} to yield the lacZ_{ON} genotype. FIG. 8B illustrates cells harboring the circuit shown in FIG. 8A were grown in the presence of different levels of aTc for 24 h at 30° C. to enable recording into genomic DNA. Subsequently, cell populations were diluted into fresh media without or with IPTG (1 mM) and incubated at 37° C. for 8 hours. Total LacZ activity in these cultures was measured using a fluorogenic lacZ substrate (FDG) assay. FIG. 8C shows a graph illustrating that total LacZ activity was elevated only at high levels of aTc and in the presence of IPTG, thus demonstrating that SCRIBE can record the magnitude of the “Input & Write” signal into an analog memory unit that is only read in the presence of a “Read” signal. FIG. 8D shows the extension of the circuit in FIG. 8A to create a sample-and-hold circuit where “Input,” “Write” and “Read” operations are independently controlled. This feature enables the creation of addressable memory registers in the genomic DNA tape. Induction of cells with the “Input” signal (AHL) produces ssDNA(lacZ)_{ON}, which targets the genomic lacZ_{OFF} locus for reversion to the wild-type sequence. In the presence of the “Write” signal (aTc), which expresses Beta, ssDNA(lacZ)_{ON} is recombined into the lacZ_{OFF} locus and produces the lacZ_{ON} genotype. Thus, the “Write” signal enables the “Input” signal to be sampled and held in memory. The total LacZ activity in the cell populations is retrieved by adding the “Read” signal (IPTG). FIG. 8E shows the induction of cells harboring the circuit shown in FIG. 8D with different combinations of aTc (100 ng/ml) and AHL (50 ng/ml) for 24 h, after which the cultures were diluted in fresh media with or without IPTG (1 mM). These cultures were then incubated at 37° C. for 8 hours and assayed for total LacZ activity with the FDG assay. FIG. 8F shows a graph illustrating a “Read” signal exhibiting enhanced levels of total LacZ activity from cell populations that received both the “Input” and “Write” signals (error bars indicate the standard error of the mean for three independent biological replicates).

### Engineered Nucleic Acid Constructs

An “engineered nucleic acid construct” refers to an engineered nucleic acid having multiple genetic elements. Engineered nucleic acid constructs of the present disclosure, in some embodiments, include a promoter operably linked to a nucleic acid that comprises (a) a nucleotide sequence encoding a single-stranded msr RNA, (b) a nucleotide sequence encoding a single-stranded msd DNA modified to contain a targeting sequence, and (c) a nucleotide sequence encoding a reverse transcriptase protein, wherein (a) and (b) are flanked by inverted repeat sequences. In some embodiments, the constructs also include a nucleotide sequence that encodes a single-stranded DNA (ssDNA)-annealing recombinase protein (e.g., a Beta recombinase protein or a Beta recombinase protein homolog). Thus, engineered constructs, as provided herein, include one or more genetic elements (e.g., promoters; retron elements that encode msr RNA, msd DNA and reverse transcriptase; inverted repeat sequences; stop codons; and/or protein-coding sequences).

Retron Elements

Aspects of the present disclosure are directed to engineered nucleic acid constructs that comprise retron-like elements. A wild-type (e.g., unmodified) retron is a type of prokaryotic retroelement responsible for the synthesis of small extra-chromosomal satellite DNA referred to as multicopy single-stranded (ms) DNA. A wild-type msDNA is composed of a small, single-stranded DNA, linked to a small, single-stranded RNA. Internal base pairing creates various stem-loop/hairpin secondary structures in the msDNA. As shown in FIG. 2A, a wild-type retron is a distinct DNA sequence that encodes a promoter, which controls the transcription of an operon that includes three loci-msr (e.g., SEQ ID NO: 6) and msd (e.g., SEQ ID NO: 7), which encode RNA moieties that serve as the primer and the template for reverse transcription, respectively, and ret (e.g., SEQ ID NO: 12), which encodes a reverse transcriptase (RT) protein. The msr-msd sequence in the retron is flanked by two inverted repeats (FIG. 2A, gray triangles). Once transcribed, the msr-msd RNA folds into a secondary structure guided by the base-pairing of the inverted repeats and the msr-msd sequence. The RT recognizes this secondary structure and uses a conserved guanosine residue in the msr as a priming site to reverse transcribe the msd sequence and produce a hybrid ssRNA-ssDNA molecule referred to as msDNA (FIG. 2A, left). As shown herein, the middle part of the msd sequence is dispensable and can be replaced with a template to produce ssDNAs of interest (e.g., see FIG. 2A, (kanR)_{ON}, right) in vivo.

In some embodiments, engineered nucleic acid constructs of the present disclosure include a DNA sequence encoding a single-stranded msr RNA, (b) a DNA sequence encoding a single-stranded msd DNA modified to contain a targeting sequence, and (c) a DNA sequence encoding a reverse transcriptase protein, wherein (a) and (b) are flanked by inverted repeat sequences. It should be understood that the DNA sequence of (b) encodes an msd RNA, which is reverse transcribed by the reverse transcriptase to produce msd DNA.

Reverse transcriptase (RT) is an enzyme used to generate complementary DNA from an RNA template. Reverse transcriptases may be obtained from prokaryotic cells or eukaryotic cells. As shown in FIG. 2A, reverse transcriptases of the present disclosure are used to reverse transcribe template msd RNA into single-stranded msd DNA. In some embodiments, a reverse transcriptase is encoded by a retron ret gene. Other examples of reverse transcriptases (RTs) that may be used in accordance with the present disclosure include, without limitation, retroviral RTs (e.g., eukaryotic cell viruses such as HIV RT and MuLV RT), group II intron RTs and diversity generating retroelements (DGRs).

An inverted repeat sequence is a sequence of nucleotides followed upstream (e.g., toward the 5′ end) or downstream (e.g., toward the 3′ end) by its reverse complement. Inverted repeat sequences of the present disclosure typically flank an msr-msd sequence in a retron and, once transcribed, binding of the two sequences guides folding of the transcribed molecule into a secondary structure. Inverted repeat sequences are typically specific for each retron. For example, an inverted repeat sequence for the wild-type retron Ec86 (or for genetic elements obtained from the type retron Ec86) is TGCGCACCCTTA (SEQ ID NO: 30). In some embodiments, the length of an inverted repeat sequence is 5 to 15, or 5 to 20 nucleotides. For example, the length of an inverted repeat sequence may be 5, 6, 7, 8, 9, 10, 11, 12, 13, 14, 15, 16, 17, 18, 19 or 20 nucleotides. In some embodiments, the length of an inverted repeat sequence is longer than 20 nucleotides.

Engineered nucleic acid constructs of the present disclosure are modified to contain a targeting sequence. A “targeting sequence” refers to a nucleotide sequence (e.g., DNA) within a single-stranded msd DNA that is complementary or partially complementary to a target sequence (e.g., genomic sequence). A targeting sequence, when bound by a ssDNA-annealing recombinase, anneals to and recombines with its target sequence. A “target sequence” may be, for example, located genomically in a cell or otherwise present in a cell (e.g., located on an episomal vector).

In some embodiments, a targeting sequence has a length of at least 15 nucleotides. For example, a targeting sequence may have a length of 15 to 100 nucleotides, or 15 to 200 nucleotides, or more. In some embodiments, a targeting sequence has a length of 15 to 50, 15 to 60, 15 to 70, 15 to 80, or 15 to 90 nucleotides. In some embodiments, a targeting sequence has a length of 20 to 50, 20 to 60, 20 to 70, 20 to 80, 20 to 90, or 20 to 100 nucleotides.

In some embodiments, a targeting sequence comprises at least 15 nucleotides (e.g., contiguous nucleotides) that are complementary to a target genomic sequence of a cell into which an engineered nucleic acid construct containing the targeting sequence has been delivered. In some embodiments, a targeting sequence comprises at least 20, at least 30, at least 40, at least 50, at least 60, at least 70, at least 80, at least 90, or at least 100 nucleotides (e.g., contiguous nucleotides) that are complementary a target genomic sequence of a cell into which an engineered nucleic acid construct containing the targeting sequence has been delivered. In some embodiments, a targeting sequence comprises 15 to 100, 15 to 90, 15 to 80, 15 to 70, 15 to 60, 15 to 50, 15 to 40, or 15 to 30 nucleotides (e.g., contiguous nucleotides) that are complementary to a target genomic sequence of a cell into which an engineered nucleic acid construct containing the targeting sequence has been delivered.

In some embodiments, a targeting sequence is 100% complementary to its target sequence. In some embodiments a targeting sequence is less that 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. Such a targeting sequence with partially complementarity to its target sequence may be used, for example, to introduce mutations or other genetic changes (e.g., genetic elements such as stop codons) into its target sequence.

A ssDNA-annealing recombinase protein, discussed below, binds to the single-stranded msd DNA and mediates annealing and recombination of the targeting sequence with its complementary, or partially-complementary, single-stranded target sequence (e.g., genomic target sequence).

In some embodiments, the retron elements of an engineered nucleic acid construct are arranged such that a promoter that is located upstream of a nucleotide sequence encoding a single-stranded msr RNA, which is located upstream of a nucleotide sequence encoding a single-stranded msd DNA modified to contain a targeting sequence, which is located upstream of a nucleotide sequence encoding a reverse transcriptase protein, wherein the nucleotide sequence encoding a single-stranded msr RNA and the nucleotide sequence encoding a single-stranded msd DNA are flanked by inverted repeat sequences (as shown in FIG. 2A). That is, in some embodiments, the retron elements of an engineered nucleic acid construct are arranged in the following 5′ to 3′ orientation: promoter, inverted repeat sequence, nucleotide sequence encoding a single-stranded msr RNA, nucleotide sequence encoding a single-stranded msd DNA, inverted repeat sequence, nucleotide sequence encoding a reverse transcriptase protein. It should be understood that each “inverted repeat sequence” is one of a pair of inverted repeat sequences that are complementary to each other and bind to each once transcribed so as to assist in folding of the transcribed RNA into a secondary structure.

In some embodiments, the retron elements of an engineered nucleic acid construct are arranged on separate nucleic acids such that the single-stranded msr RNA and the single-stranded msd DNA are encoded in trans with the reverse transcriptase. For example, one engineered nucleic acid construct may comprise a promoter is located upstream of a nucleotide sequence encoding a single-stranded msr RNA, which is located upstream of a nucleotide sequence encoding a single-stranded msd DNA modified to contain a targeting sequence, wherein the nucleotide sequence encoding a single-stranded msr RNA and the nucleotide sequence encoding a single-stranded msd DNA are flanked by inverted repeat sequences, and another engineered genetic construct may comprise a promoter located upstream of a nucleotide sequence encoding a reverse transcriptase protein. That is, in some embodiments, the retron elements of one engineered nucleic acid construct are arranged in the following 5′ to 3′ orientation: promoter, inverted repeat sequence, nucleotide sequence encoding a single-stranded msr RNA, nucleotide sequence encoding a single-stranded msd DNA, inverted repeat sequence. In such embodiments, another engineered nucleic acid construct contains a promoter 5′, or upstream, relative to a nucleotide sequence encoding a reverse transcriptase protein.

ssDNA-Annealing Recombinase Proteins

Recombination of ssDNA produced in vivo may be mediated by a ssDNA-annealing recombinase protein. Thus, aspects of the present disclosure are directed to engineered nucleic acid constructs that encode, and cells that comprise, single-stranded DNA (ssDNA)-annealing recombinases such as, for example, Beta recombinase protein (e.g., encoded by the bacteriophage lambda bet gene) or a homolog thereof. When expressed in cells (e.g., bacterial cells such as Escherichia coli cells) ssDNA-annealing recombinases mediate ssDNA recombination. The term “recombination” refers to the process by which two nucleic acids exchange genetic information (e.g., nucleotides). Non-limiting examples of ssDNA-annealing recombinases for use in accordance with the present disclosure include recombinases obtained from bacteriophages or prophages of Gram-positive bacteria Bacillus subtilis, Mycobacterium smegmatis, Listeria monocytogenes, Lactococcus lactis, Staphylococcus aureus, and Enterococcus faecalis as well as from the Gram-negative bacteria Vibrio cholerae, Legionella pneumophila, and Photorhabdus luminescens (S. Datta, et al. PNAS 105, 1616-1631 (2008)). Specific examples of recombinases for use as provided herein include, without limitation, those listed in Table 5.

TABLE 5
ssDNA-Annealing Recombinase Proteins

| | | | | Recombinase (R) |
|---|---|---|---|---|
| Original | | Accession | | Exonuclease (E) genes and |
| Host | Source | Number | Nucleotide | promoter (P) |
| bet/exo | Phage lambda; | NIH | NC_001416 | 32025-32810/31348- |
| | E. coli | collection | | 32028 |
| s065/s066 | SXT element; | D. I. | AY055428 | 72817-73635/73921- |
| | Vibrio choleras | Friedman | | 74937 |
| plu2935/ | Photorhabdus | A. Danchin | BX571868 | 324693-325613/325614- |
| plu2936 | luminescens | | | 326297 |
| EF2132/ | Enterococcus | S. L. Adhya | AE016830 | 2041370-2042293/2040592- |
| EF2131 | faecalis | | | 2041404 |
| recT/recE | Rac prophage; | NIH | NC_000913 | 1412008-1412817/1412810- |
| | E. coli | collection | | 1415410 |
| orfC/orfB | Legionella | E. Lüneberg | AJ277755 | 1415-2299/560-1402 |
| | pneumophila | | | |
| gp35/ | Phage SPP1; | S. Moineau | X97918 | 32175-33038/30532- |
| gp34.1 | Bacillus subtilis | | | 31467 |
| gp61/gp60 | Phage Che9c; | G. Hatfull | AY129333 | 43643-44704/42706- |
| | Mycobacterium | | | 43650 |
| | smegmatis | | | |
| orf48/ | Phage A118; | R. Calender | AJ242593 | 32773-33588/31811- |
| orf47 | Listeria monocytogenes | | | 32770 |
| orf245/— | Phage ul36.2; | S. Moineau | AF212847 | 1678-2415 |
| | Lactococcus lactis | | | |
| gp20/— | Phage phiNM3; | T. Bae | NC_008617 | 10317-11237 |
| | Staphylococcus aureus | | | |

Bacteriophage lambda Red Beta recombinase protein (referred to herein as “Beta recombinase”) (e.g., SEQ ID NO: 13) mediates recombination-mediated genetic engineering, or “recombineering,” using ssDNA. Unlike recombineering with double-stranded DNA, recombineering with ssDNA does not require other bacteriophage lambda red recombination proteins, such as Exo and Gamma. Beta recombinase binds to ssDNA and anneals the ssDNA to complementary ssDNA such as, for example, complementary genomic DNA. It can efficiently recombine linear DNA with homologs as short, for example, 20-70 bases (N. Constantino et al., PNAS USA 100(26): 15748-53 (2003)). Thus, in some embodiments, as discussed above, a targeting sequence has a length of 20 to 70 nucleotides. As used herein, the term “Beta recombinase,” in some embodiments, may include Beta recombinase homologs (S. Datta, et al. Proc Natl Acad Sci USA 105: 1626-1631 (2008)), in addition to the recombinases listed in Table 5.

Nucleic Acids

A “nucleic acid” refers to at least two nucleotides covalently linked together, and in some instances, may contain phosphodiester bonds (e.g., a phosphodiester “backbone”). In some embodiments, a nucleic acid (e.g., an engineered nucleic acid) of the present disclosure may be considered a nucleic acid analog, which may contain other backbones comprising, for example, phosphoramide, phosphorothioate, phosphorodithioate, O-methylphophoroamidite linkages, and/or peptide nucleic acids. Nucleic acids (e.g., components, or portions, of the nucleic acids) of the present disclosure may be naturally occurring or engineered. Nucleic acids of the present disclosure may be single-stranded (ss) or double-stranded (ds), as specified, or may contain portions of both single-stranded and double-stranded sequence (e.g., a single-stranded nucleic acid with stem-loop structures may be considered to contain both single-stranded and double-stranded sequence). It should be understood that a double-stranded nucleic acid is formed by hybridization of two single-stranded nucleic acids to each other. Nucleic acids may be DNA, including genomic DNA and cDNA, RNA or a hybrid/chimeric of any two or more of the foregoing, where the nucleic acid contains any combination of deoxyribo- and ribonucleotides, and any combination of bases, including uracil, adenine, thymine, cytosine, guanine, inosine, xanthine, hypoxanthine, isocytosine, and isoguanine.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing.

Engineered nucleic acid constructs of the present disclosure may be encoded by a single molecule (e.g., included in the same plasmid or other vector) or by multiple different molecules (e.g., multiple different independently-replicating molecules).

Engineered nucleic acid constructs of the present disclosure may be produced using standard molecular biology methods (see, e.g., Green and Sambrook, Molecular Cloning, A Laboratory Manual, 2012, Cold Spring Harbor Press).

In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Engineered nucleic acid constructs of the present disclosure may be included within a vector, for example, for delivery to a cell. A “vector” refers to a nucleic acid (e.g., DNA) used as a vehicle to artificially carry genetic material (e.g., an engineered nucleic acid construct) into a cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

Promoters

Engineered nucleic acid constructs of the present disclosure may contain promoters operably linked to a nucleic acid containing sequences that encode, for example, retron elements and/or recombinases. A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be classified as strong or weak according to its affinity for RNA polymerase (and/or sigma factor); this is related to how closely the promoter sequence resembles the ideal consensus sequence for the polymerase. The strength of a promoter may depend on whether initiation of transcription occurs at that promoter with high or low frequency. Different promoters with different strengths may be used to engineer nucleic acids with different levels of gene/protein expression (e.g., the level of expression initiated from a weak promoter is lower than the level of expression initiated from a strong promoter).

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. No. 4,683,202 and U.S. Pat. No. 5,928,906).

Examples of promoters for use in accordance with the present disclosure include, without limitation, P_{lacO} (e.g., SEQ ID NO: 1), P_{tetO} (e.g., SEQ ID NO: 6), P_{luxR} (e.g., SEQ ID NO: 3), P_{λR} (e.g., SEQ ID NO: 4) and P_{fixK2} (e.g., SEQ ID NO: 5). Other promoters are described below.

Inducible Promoters

Promoters of an engineered nucleic acid construct may be “inducible promoters,” which refer to promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, an inducer signal of the present disclosure is an N-acyl homoserine lactone (AHL), which is a class of signaling molecules involved in bacterial quorum sensing. Quorum sensing is a method of communication between bacteria that enables the coordination of group based behavior based on population density. AHL can diffuse across cell membranes and is stable in growth media over a range of pH values. AHL can bind to transcriptional activators such as LuxR and stimulate transcription from cognate promoters.

In some embodiments, an inducer signal of the present disclosure is anhydrotetracycline (aTc), which is a derivative of tetracycline that exhibits no antibiotic activity and is designed for use with tetracycline-controlled gene expression systems, for example, in bacteria.

Other inducible promoter systems are known in the art and may be used in accordance with the present disclosure.

In some embodiments, inducible promoters of the present disclosure function in prokaryotic cells (e.g., bacterial cells). Examples of inducible promoters for use prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), σS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacOl, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and aB promoters. Other inducible microbial promoters may be used in accordance with the present disclosure.

In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

Stop Codons

Engineered nucleic acid constructs of the present disclosure, in some embodiments, comprise a genetic element that prevents translation of a downstream product (e.g., reporter molecule). In some embodiments, the genetic element is a stop codon. A stop codon is a nucleotide triplet within RNA that signals termination of translation. In some embodiments, an engineered nucleic acid constructs comprises more than one stop codon (e.g., 2 or 3 stop codons). Examples of standard stop codons include, without limitation, UAG, UAA and UGA in RNA, and TAG, TAA and TGA in DNA. Other genetic elements that prevent translation of a downstream product are contemplated herein.

### Cells and Cell Expression

Engineered nucleic acid constructs of the present disclosure may be expressed in a broad range of host cell types. In some embodiments, engineered constructs are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the invention are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, engineered nucleic acid constructs are expressed in mammalian cells. For example, in some embodiments, engineered nucleic acid constructs are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature (e.g., an engineered nucleic acid encoding a ssDNA-annealing recombinase protein such as Beta recombinase protein). In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination). In some embodiments, a cell overexpresses genes encoding the subunits of Exo VII of Escherichia coli. Thus, in some embodiments, a cell overexpressed one or more genes encoding XseA and/or XseB of Escherichia coli or homologs thereof.

In some embodiments, a cell contains a gene deletion. For example, the present disclosure contemplates modified bacterial cells, such as modified Escherichia coli bacterial cells that lack genes encoding RecJ and/or XonA, which are exonucleases. In some embodiments, modified bacterial cells lack one or more other exonucleases.

In some embodiments, an engineered nucleic acid construct may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acid constructs of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

### Methods

Aspects of the present disclosure provide methods that include delivering to cells at least one of the engineered nucleic acid constructs as provided herein. Constructs may be delivered by any suitable means, which may depend on the residence and type of cell. For example, if cells are located in vivo within a host organism (e.g., an animal such as a human), engineered nucleic acid constructs may be delivered by injection into the host organism of a composition containing engineered nucleic acid constructs. Constructs may be delivered by a vector, such as a viral vector (e.g., bacteriophage or phagemid). For cells that are not located within a host organism, for example, for cells located ex vivolin vitro or in an environmental (e.g., outside) setting, engineered nucleic acid constructs may be delivered to cells by electroporation, chemical transfection, fusion with bacterial protoplasts containing recombinant, transduction, conjugation, or microinjection of purified DNA directly into the nucleus of the cells.

Cells to which engineered nucleic acid constructs are delivered typically contain a nucleotide sequence, referred to as a “target sequence,” which is complementary to the targeting sequence of the construct. A target sequence may be located within the genome of the cell, or the target sequence may be located episomally (e.g., on a plasmid) within the cell. In some embodiments, a target sequence is located in an engineered nucleic acid construct. For example, one engineered nucleic acid construct may contain a nucleic acid encoding a targeting sequence that is complementary (or partially complementary) to a target sequence located in another engineered nucleic acid construct.

In some embodiments, a cell comprises a ssDNA-annealing recombinase protein (e.g., an endogenous ssDNA-annealing protein such as an endogenous Beta recombinase protein). Thus, in some embodiments, methods comprise delivering to such cells engineered nucleic acid constructs that do not encode a ssDNA-annealing recombinase protein. In some embodiments, a cell does not comprise a ssDNA-annealing recombinase protein. Thus, in some embodiments, methods comprise delivering to such cells engineered nucleic acid constructs that encode a ssDNA-annealing recombinase protein. In some embodiments, for example, where a cell does not contain a ssDNA-annealing recombinase protein, methods may comprise delivering to cells (a) at least one of the engineered nucleic acid constructs as provided herein that does not encode a ssDNA-annealing recombinase protein, and (b) an engineered nucleic acid construct comprising a promoter operably linked to a nucleic acid encoding a single-stranded DNA (ssDNA)-annealing recombinase protein.

In some embodiments, methods comprise exposing cells that contain engineered nucleic acid constructs as provided herein to at least one signal that regulates transcription of at least one nucleic acid of a construct. A signal that regulates transcription of nucleic acid may be a signal (e.g., chemical or non-chemical) that activates, inactivates or otherwise modulates transcription of a nucleic acid. For transcription of a nucleic acid of an engineered nucleic acid construct of the present disclosure to be regulated, conditions under which cells are exposed should permit transcription. Such conditions will depend on the cells and the genetic elements used to construct the engineered nucleic acid constructs (e.g., exposing cells to signals (e.g., chemical or non-chemical conditions) known to regulate transcription of particular inducible promoters).

In some embodiments, a cell that contains engineered nucleic acid constructs is exposed more than once to a signal that regulates transcription of a nucleic acid of an engineered nucleic acid construct as provided herein. For example, a cell may be exposed to a signal 2, 3, 4, 5, 6, 7, 8, 9, 10 or more times. The cell exposure may occur over the period of minutes (e.g., 5, 10, 15, 20, 25, 30, 35, 40, 45, 50 or 55 minutes), hours (e.g., 1, 2, 3, 4, 5, 6, 7, 8, 9 10, 11, 12, 13, 14, 15, 16, 17, 18, 19, 20, 21, 22 or 23 hours), days (e.g., 2, 3, 4, 5 or 6 days), weeks (e.g., 1, 2, 3 or 4 weeks), or months (1, 2, 3, 4, 5, 6, 7, 8, 9, 10, 11 or 12 months), or for a shorter or longer duration. Cell exposure may be at regular intervals or intermittently.

In some embodiments, a signal that activates transcription is an endogenous signal, meaning that the signal is generated from within the cell or by the cell. For example, cell exposure to certain environmental conditions may cause the cell to produce, intracellularly or extracellular, a chemical or non-chemical signal that activates transcription of a nucleic acid of an engineered nucleic acid construct of the present disclosure.

In some embodiments, cells that contain one or more engineered nucleic acid construct of the present disclosure are permitted to express the constructs (e.g., incubated at conditions suitable for cell expression) for a prolonged period of time (e.g., at least 2 days, at least 3 days, at least 4 days, at least 5 days, at least 6 days, at least 7 days, at least 8 days, at least 9 days, at least 10 days, or more).

In some embodiments, cells that express the Exo VII complex and contain one or more engineered nucleic acid construct of the present disclosure are permitted to express the constructs for a shortened period of time (e.g., less than 2 days, less than 1 day, or less than 12 hours).

### Applications

In some embodiments, methods and composition of the present disclosure may be used for in vivo genome editing, which enables the construction of scalable DNA memory in live cells. For example, SCRIBE may be used to create long-term “recorders” for environmental and biomedical applications where a population of engineered bacteria is harvested at periodic time points to determine the history of exposure to signals of interest. Thus, in some embodiments, provided herein are methods of delivering to engineered bacterial cells an engineered nucleic acid construct comprising a promoter operably linked to a nucleic acid that comprises (a) a nucleotide sequence encoding a single-stranded msr RNA, (b) a nucleotide sequence encoding a single-stranded msd DNA modified to contain a targeting sequence, and (c) a nucleotide sequence encoding a reverse transcriptase protein, wherein (a) and (b) are flanked by inverted repeat sequences. In some embodiments, the engineered bacterial cells comprise a genomic locus that has been modified to express a reporter molecule. In some embodiments, the targeting sequence is partially complementary to a genomic sequence (e.g., a sequence with a modified locus) of the engineered bacterial cells.

As another example, the memory units can be linked to quorum-sensing circuits to implement a population-level biosensor that triggers a response only when the population-encoded memory reaches a predetermined threshold. Moreover, the ability to introduce diversity within subpopulations of clonal populations may be used to engineer multicellular consortia for distributed computing (W. Bacchus, et al. Metab Eng 16, 33-41 (2013)). Combining SCRIBE with analog computing circuits (R. Daniel, et al. Nature 497, 619-623 (2013)) may further increase the dynamic range for analog memory in living cells and realize complex analog-memory-and-computation circuits. Additional modifications to the SCRIBE platform (e.g., by suppressing a host's mismatch repair system (N. Costantino, et al. Proc Natl Acad Sci USA 100, 15748-15753 (2003)) can be made to provide more efficient DNA memory, which enables other applications, including, for example, dynamic engineering of cellular phenotypes and the construction of complex cellular state machines and biological Turing machines (Y. Benenson, Nat Rev Genet 13, 455-468 (2012); Y. Benenson, et al. Nature 414, 430-434 (2001); K. Oishi, et al. ACS Synthetic Biology, (2014)).

In vivo ssDNA expression also enhances the efficiency of genome engineering and expands the applicability of multiplexed recombineering strategies beyond standard lab strains. Recombineering approaches, such as Multiplex Automated Genome Engineering (MAGE) (H. H. Wang, et al. Nature 460, 894-898 (2009)), rely on high-efficiency electroporation of recombinogenic oligonucleotides into cells to perform targeted mutagenesis. However, high-efficiency transformation is not achievable in many strains or species of interest. Because retrons have been found in a diverse range of microorganisms (B. C. Lampson, et al. Cytogenetic and genome research 110, 491-499 (2005)) and have been shown to be functional in eukaryotes as well (J. R. Mao, et al. J Biol Chem 270, 19684-19687 (1995); O. Mirochnitchenko, et al. J Biol Chem 269, 2380-2383 (1994); S. Miyata, et al. Proc Natl Acad Sci USA 89, 5735-5739 (1992)), applications based on in vivo ssDNA expression may be extended to many organisms. For example, the approach for ssDNA generation and genomic mutagenesis within living cells, as provided herein, can be encoded on plasmids, which can be introduced into target cells with high efficiency by conjugation or transduction. Thus, recombineering with ssDNAs expressed in vivo can be extended to hard-to-transform microorganisms where Beta and its homologs are functional. Furthermore, by using error-prone RNA polymerases (S. Brakmann, et al. Chembiochem 2, 212-219 (2001)) and reverse transcriptases (K. Bebenek, et al. J Biol Chem 264, 16948-16956 (1989); J. D. Roberts, et al. Science 242, 1171-1173 (1988)), mutagenized ssDNA libraries can be generated in vivo. This pool of ssDNAs can then be targeted to desired loci a within cell population. This in vivo diversity generation platform can then be placed under a gradually increasing selection pressure, to increase rate of evolution at specific sites of a genome, which can be used, for example, for continuous direct evolution of phenotypes of interest. In vivo targeted diversity generation can also enable platforms for in vivo cellular barcoding and continuous adaptive evolution (K. M. Esvelt, et al. Nature 472, 499-503 (2011)).

In addition, SCRIBE DNA memory can be extended to organisms with active ssDNA recombination machineries, such as yeast (J. R. Simon, et al. Mol Cell Biol 7, 2329-2334 (1987); J. E. Dicarlo, et al. ACS Synth Biol, (2013)) and human cells (X. Rios, et al. PLoS One 7, e36697 (2012)). Moreover, homology-directed repair and recombination pathways can be activated by introducing targeted double-stranded breaks (or nicks) into genomic DNA of both eukaryotes and prokaryotes (L. Davis, et al. Proc Natl Acad Sci USA 111, E924-932 (2014); W. Mandecki, Proc Natl Acad Sci USA 83, 7177-7181 (1986); G. A. Cromie, et al. Mol Cell 8, 1163-1174 (2001); F. A. Ran, et al. Cell 154, 1380-1389 (2013)). These data suggest that DNA memory based on the in vivo expression of ssDNAs (using retrons, retroviral RTs, or other classes of RTs) can be used in higher eukaryotes, for example, in combination with technologies such as CRISPR nucleases (F. A. Ran, et al. Cell 154, 1380-1389 (2013); L. Cong, et al. Science 339, 819-823 (2013); P. Mali, et al. Science 339, 823-826 (2013). For example, in vivo ssDNAs can be combined with inducible guide RNAs (e.g. expressed from RNA polymerase II-dependent promoters for CRISPR/Cas9 nucleases in order to introduce defined mutations and store DNA memory in the genomes of human cells. This platform can be used to record exogenous and endogenous regulatory signals (e.g., neural activity (A. Chaudhuri, Neuroreport 8, v-ix (1997)) in the genomic DNA of human cells, which can then be read at a later time using high-throughput sequencing (see, e.g., Example 12) to map the temporal nature of complex networks. Furthermore, in some instances, this system can be used to introduce conditional genetic changes into target genes with tissue-specific and/or spatiotemporal control. SCRIBE's ability to elevate the mutation rate of specific genomic sites in response to external signals also offers a valuable tool for the study of evolution and population dynamics, where traditional approaches are limited by low mutation rates and the restricted timescales of laboratory evolution studies (T. J. Kawecki, et al. Trends Ecol Evol 27, 547-560 (2012)).

Further, in vivo ssDNA generation can be used to create DNA nanostructures and nanorobots (Y. Amir, et al. Nat Nanotechnol 9, 353-357 (2014); L. Qian, et al. Nature 475, 368-372 (2011); G. Seelig, et al. Science 314, 1585-1588 (2006); P. W. Rothemund, Nature 440, 297-302 (2006); S. M. Douglas, et al. Nature 459, 414-418 (2009); S. M. Douglas, et al. Science 335, 831-834 (2012); S. M. Chirieleison, et al. Nat Chem 5, 1000-1005 (2013)) that can probe and modulate the behavior of living cells or enable the construction of scalable and dynamic ssDNA-protein hybrid nanomachines with novel functionalities in living cells (C. A. Brosey, et al. Nucleic Acids Res 41, 2313-2327 (2013)). In addition, the bacterial ssDNA expression system of the present disclosure can be modified and scaled-up to create an economical source of ssDNAs for DNA nanotechnology (S. Kosuri, et al. Nat Methods 11, 499-507 (2014)). In summary, the in vivo ssDNA production and SCRIBE platforms provided herein open up a broad range of new capabilities for, e.g., biomedical research, synthetic biology, genome engineering and DNA nanotechnology in a wide variety of organisms.

### EXAMPLES

### Example 1

The expression of Beta recombinase from bacteriophage λ in Escherichia coli (E. coli) promotes high levels of oligonucleotide-mediated recombination (N. Costantino, et al. Proc Natl Acad Sci USA 100, 15748-15753 (2003); J. A. Sawitzke, et al. J Mol Biol 407, 45-59 (2011); S. K. Sharan, et al. Nat Protoc 4, 206-223 (2009); B. Swingle, et al. Mol Microbiol 75, 138-148 (2010)). Synthetic oligonucleotides delivered by electroporation into cells that overexpress Beta are specifically and efficiently recombined into homologous genomic sites. Thus, oligonucleotide-mediated recombineering offers a powerful way to introduce targeted mutations in a bacterial genome. However, this technique requires the exogenous delivery of ssDNAs and cannot be used to couple arbitrary signals into genetic memory.

To precisely write genetic information into genomes in response to arbitrary signals and without the need for exogenous oligonucleotides, provided herein is a genome-editing platform based on expressing ssDNAs inside of living cells. To express ssDNA in vivo, a widespread class of bacterial reverse transcriptases, referred to as retrons (T. Yee, et al. Cell 38, 203-209 (1984); B. C. Lampson, et al. Cytogenetic and genome research 110, 491-499 (2005)), were used. The wild-type retron cassette encodes three components in a single transcript—a reverse transcriptase protein (RT) and two RNA moieties, msr and msd, which act as the primer and the template for the reverse transcriptase, respectively (FIG. 2A, left). To couple the expression of ssDNA to an external input, the retron Ec86 cassette (D. Lim, et al. Cell 56, 891-904 (1989)) was placed under the control of the P_{lacO} promoter (FIG. 2A, left), which can be induced by Isopropyl β-D-1-thiogalactopyranoside (IPTG), and transformed the construct into E. coli K-12 DH5αPRO (R. Lutz, et al. Nucleic Acids Res 25, 1203-1210 (1997)), which expresses high levels of the Lad and TetR repressors. As shown in FIG. 2B, the wild-type retron ssDNA (ssDNA(wt)) was readily detected in IPTG-induced cells while no ssDNA was detected in non-induced cells, thus demonstrating tight regulation. The identity of the detected ssDNA band was further confirmed by DNA sequencing. To verify that ssDNA expression depends on RT activity, point mutations (D197A and D198A) were introduced to the active site of the RT to make a catalytically dead RT (dRT) (P. L. Sharma, et al. Antivir Chem Chemother 16, 169-182 (2005)). This modification completely abolished ssDNA production (FIG. 2B), confirming that ssDNA production depends on RT activity.

### Example 2

The msd template was engineered to express synthetic ssDNAs of interest. The msd(wt) RNA is predicted to form a stable stem-loop structure (D. Lim, et al. Cell 56, 891-904 (1989)), as depicted in FIG. 2A. Initially, the whole msd sequence was replaced with a desired template. However, no ssDNA was detected (data not shown), suggesting that some features of msd are required for ssDNA expression, as previously noted for another retron (J. R. Mao, et al. J Biol Chem 270, 19684-19687 (1995)). Therefore, different positions along the msd sequence were tested for insertion. A variant in which the flanking regions of the msd stem remained intact (FIG. 2A, right) produced detectable amounts of ssDNA when induced by IPTG (FIG. 2B, P_{lacO}_{_}msd(kanR)_{ON}+IPTG). The correct identity of the detected ssDNA band was further confirmed by DNA sequencing. These results suggest that the lower part of the msd stem is essential for reverse transcription while the upper part of the stem and the loop are dispensable and can be replaced with desired ssDNA templates.

### Example 3

To demonstrate that intracellularly expressed ssDNAs can be recombined into target genomic loci by concomitant expression of Beta (N. Costantino, et al. Proc Natl Acad Sci U SA 100, 15748-15753 (2003); J. A. Sawitzke, et al. J Mol Biol 407, 45-59 (2011); S. K. Sharan, et al. Nat Protoc 4, 206-223 (2009); B. Swingle, et al. Mol Microbiol 75, 138-148 (2010)), a selectable marker reversion assay was developed (FIG. 2C). The kanR gene, which encodes neomycin phosphotransferase II and confers resistance to kanamycin (Kan), was integrated into the galK locus through recombineering. Two stop codons were then introduced into the genomic kanR to make a Kan-sensitive kanR_{OFF} reporter strain (DH5αPRO galK::kanR_{W28TAA, A29TAG}). These premature stop codons could be reverted back to the wild-type sequence through recombination with engineered ssDNA(kanR)_{ON}, thus conferring kanamycin resistance (FIG. 2A-D). Specifically, ssDNA(kanR)_{ON} contains 74 base pairs (bp) of homology to the regions of the kanR_{OFF} locus flanking the premature stop codons, and replaces the stop codons with the wild-type kanR gene sequence (FIG. 2D; SEQ ID NO: 36 (top), SEQ ID NO: 37 (bottom)). In this assay, the recombinant frequency (the ratio between the number of Kan-resistant cells to the total number of viable cells) in a culture is used to measure the efficiency of recombination.

The Beta gene (bet) was cloned into a plasmid under the control of the anhydrotetracycline (aTc)-inducible P_{tetO} promoter and introduced it along with the IPTG-inducible msd(kanR)_{ON} construct into the kanR_{OFF} strain (FIG. 2C). As shown in FIG. 2E, induction of cultures harboring these two plasmids with either IPTG or aTc resulted in a slight increase in the number of the Kan-resistant cells. However, co-expression of both ssDNA(kanR)_{ON} and Beta with IPTG and aTc resulted in a >10⁴-fold increase in the recombinant frequency relative to the non-induced cells. This increase in the recombinant frequency was dependent on RT activity, as it was abolished with dRT (FIG. 2E). The genotypes of randomly selected Kan-resistant colonies were further confirmed by DNA sequencing to contain precise reversions of the two codons to the wild-type sequence. No Kan-resistant colonies were detected when a non-specific ssDNA (ssDNA(wt)) was co-expressed with Beta in the kanR_{OFF} reporter cells, confirming that Kan-resistant cells were not produced due to spontaneous mutations. These results show that the presence of an arbitrary input (e.g., IPTG) can be successfully recorded in genomic DNA through precise in vivo genome editing.

### Example 4

Epigenetic and recombinase-based memory devices have limited storage capacities because they have digital responses, rapidly saturate the proportion of cells carrying a specific state, and have not fully leveraged the genomic DNA capacity within the large numbers of cells in a bacterial culture. Thus, these devices have been largely limited to recording binary information, such as the presence of inputs, and have not been used to record analog information, such as the magnitude of inputs. Herein, it was shown that the recombination rate between engineered ssDNAs and genomic DNA can be effectively modulated by changing expression levels of an engineered retron cassette and Beta. This feature enables the recording of analog information, such as the magnitude of an input signal, in the proportion of cells in a population with a specific mutation in genomic DNA. This was demonstrated by placing both the ssDNA(kanR)_{ON} expression cassette and bet into a single synthetic operon (hereafter referred to as the SCRIBE(kanR)_{ON} cassette) under the control of P_{lacO} (FIG. 2F). The kanR_{OFF} reporter cells harboring this synthetic operon were induced with different concentrations of IPTG. As shown in FIG. 2G, the fraction of Kan-resistant recombinants increased linearly with the input inducer concentration on a log-log plot. Thus, SCRIBE can store the magnitude of transcriptional inputs into DNA memory in an analog fashion, and the memory can be read out by analyzing allele frequencies in the population.

### Example 5

SCRIBE records memory by using homology-based addresses to recombine ssDNA directly into genomic DNA (FIG. 1C), thus, it can be used to write arbitrary DNA information de novo into target loci. This feature contrasts with recombinase-based memory, which can only manipulate larger stretches of DNA located within pre-existing specific recombinase-recognition sites. For example, this Example shows that SCRIBE can write DNA mutations into a target loci and then reset the mutations to the original sequence using a selectable/counterselectable galK assay (S. Warming, et al. Nucleic Acids Res 33, e36 (2005)). Cells expressing galK can metabolize and grow on galactose as the sole carbon source. However, these galK-positive (galK_{ON}) cells cannot metabolize 2-deoxy-galactose (2DOG) and cannot grow on plates containing glycerol (carbon source)+2DOG. On the other hand, galK-negative (galK_{OFF}) cells cannot grow on galactose as the sole carbon source but can grow on glycerol+2DOG plates. DH5αPRO galK_{ON} cells were transformed with plasmids expressing IPTG-inducible SCRIBE(galK)_{OFF} and aTc-inducible SCRIBE(galK)_{ON} cassettes (FIG. 3A). Induction of SCRIBE(galK)_{OFF} by IPTG resulted in the writing of two stop codons into galK_{ON}, leading to galK_{OFF} cells that could grow on glycerol+2DOG plates (FIG. 3B-C). Induction of SCRIBE(galK)_{ON} in these galK_{OFF} cells with aTc reversed the IPTG-induced modification, leading to galK_{ON} cells that could grow on galactose plates (FIGS. 3B and D). These results show that in vivo writing in genomic DNA is reversible and that distinct information can be written and rewritten into the same locus.

### Example 6

Scaling the capacity of previous memory devices is challenging because each additional bit of information requires new orthogonal proteins (e.g., recombinases or transcription factors). In contrast, orthogonal SCRIBE memory devices are easier to scale because they can be built by simply reprogramming the ssDNA template (msd). To demonstrate this, SCRIBE was multiplexed to record multiple independent inputs into different genomic loci. The kanR_{OFF} reporter gene was integrated into the bioA locus of DH5αPRO to create a kanR_{OFF} galK_{ON} strain. These cells were then transformed with plasmids expressing IPTG-inducible SCRIBE(kanR)_{ON} and aTc-inducible SCRIBE(galK)_{OFF} cassettes (FIG. 3E). Induction of these cells with IPTG or aTc resulted in the production of cells with phenotypes corresponding to kanR_{ON} galK_{ON} or kanR_{OFF} galK_{OFF} genotypes, respectively (FIGS. 3F and G). Comparable numbers of kanR_{ON} galK_{ON} and kanR_{OFF} galK_{OFF} cells were produced when the cultures were induced with both aTc and IPTG (FIG. 3G). Furthermore, very few individual colonies containing both writing events (kanR_{ON} galK_{OFF}) were obtained in the cultures that were induced with both aTc and IPTG (Figure FIGS. 4A-4B). Thus, SCRIBE can be multiplexed by simply expressing different ssDNA templates and two independent inputs can be successfully recorded into genomic DNA within bacterial subpopulations. This finding enables targeted in vivo genome editing with specific mutations and has the potential to expand the capacity of DNA memory devices since the entire genome may be accessible for the dynamic storage of information.

### Example 7

In SCRIBE, the expression of each individual ssDNA can be triggered by any endogenous or exogenous signal that can be coupled into transcriptional regulation, thus recording these inputs into long-lasting DNA storage. In addition to small-molecule chemicals (FIG. 2 and FIG. 3), the present disclosure shows that light can be used to trigger specific genome editing for genomically-encoded memory. The SCRIBE(kanR)_{ON} cassette was placed under the control of a previously described light-inducible promoter (P_{Dawn}, (R. Ohlendorf, et al. J Mol Biol 416, 534-542 (2012)) within kanR_{OFF} cells (FIG. 5A). These cultures were then grown for 4 days in the presence of light or in the dark (FIGS. 5B and 5C). At the end of each day, dilutions of these cultures were made into fresh media and samples were also taken to determine the number of Kan-resistant and viable cells (FIG. 5C). Cultures grown in the dark yielded undetectable levels of Kan-resistant cells (FIG. 5D). In contrast, the number of Kan-resistant colonies increased steadily over time in the cultures that were grown in the presence of light, indicating the successful recording of light input into long-lasting DNA memory. The analog memory faithfully stored the total time of light exposure, rather than just the digital presence or absence of light. This is the first example of using light for precise genome editing and DNA memory in living cells.

### Example 8

The linear increase in the number of Kan-resistant colonies over time due to exposure to light indicates that the duration of inputs can be recorded into population-wide DNA memory using SCRIBE. To further demonstrate population-wide genomically encoded memory whose state is a function of input exposure time, the kanR_{OFF} strain harboring the constructs shown in FIG. 2C were used, where expression of ssDNA(kanR)_{ON} and Beta are controlled by IPTG and aTc, respectively. These cells were subjected to four different patterns of inputs for 12 successive days (patterns I-IV, FIG. 5E). As shown in FIG. 5F, accumulation of Kan-resistant cells was not observed in the negative control (pattern I), which was never exposed to the inducers. The fraction of Kan-resistant cells in the three other patterns (II, III, and IV) increased linearly over their respective induction periods and remained relatively constant when the inputs were removed. These data indicate that the genomically encoded memory is stable in the absence of the inputs over the course of the experiment. Notably, the recombinant frequencies in patterns III and IV, which were induced for the same total amount of time but with different temporal patterns, reached comparable levels at the end of the experiment. These data demonstrate that the genomic memory integrates over the total induction time and is independent of the input pattern, and therefore can be used to stably record long-term event histories (e.g., over many days).

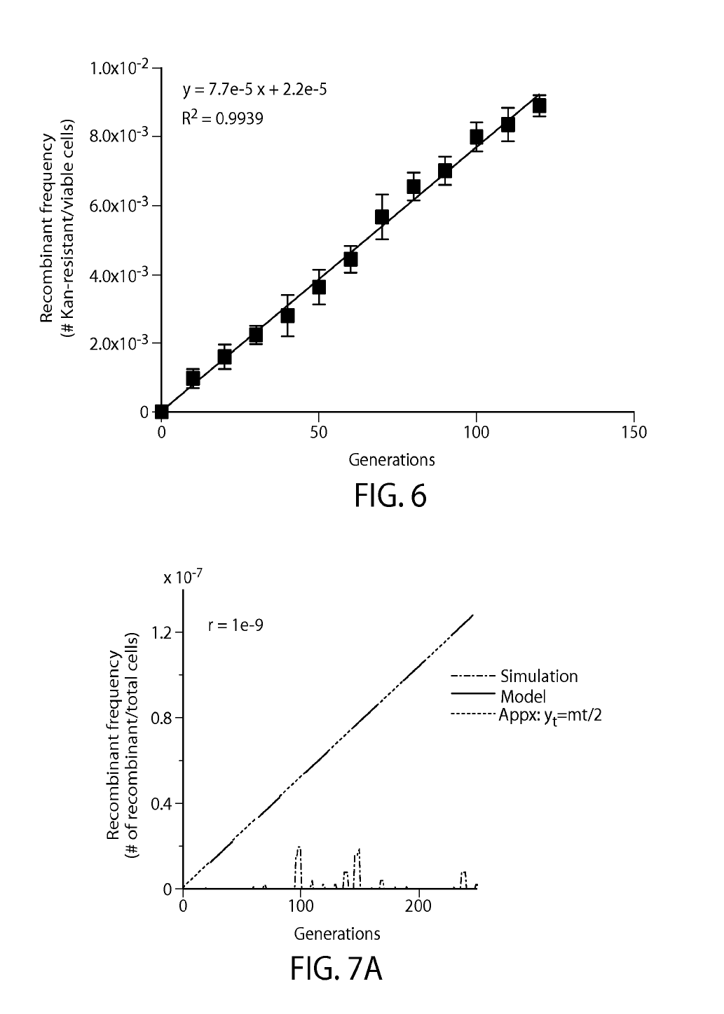

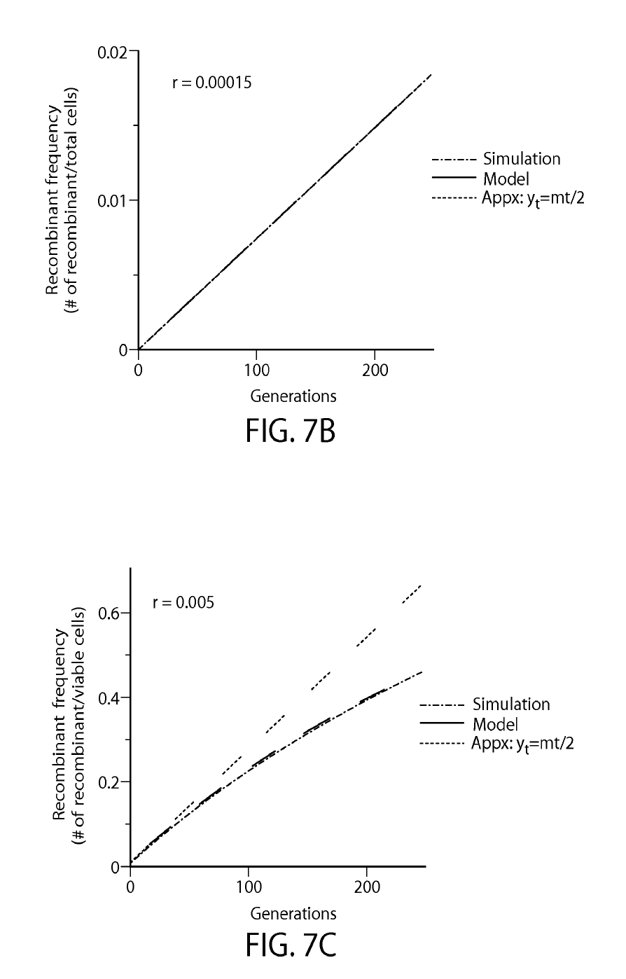

The linear increase in the fraction of recombinants in the induced cell populations over time was consistent with a deterministic model (dashed lines in FIG. 5, see below). Specifically, when triggered by inputs, SCRIBE can significantly increase the rate of recombination events at a specific target site above the wild-type rate (which is <10⁻¹⁰ events/generation in recA-background (B. E. Dutra, et al. Proc Natl Acad Sci USA 104, 216-221 (2007)). When recombination rates are ˜10⁴ events/generation, which is consistent with the recombination rate estimated for SCRIBE from data in FIG. 5F, a simple deterministic model as well as a detailed stochastic simulation both predict a linear increase in the total number of recombinant alleles in a population over time, as long as the frequency of recombinants in the population is less than a few percent and cells in the population are equally fit over the time scale of interest (below and FIGS. 6 and 7A-7B). This feature enables SCRIBE to be used as a population-level distributed memory system to store analog memory values that integrate the time span over which cells are induced.

### Example 9

Both ssDNA expression and Beta are required for writing into genomic memory (FIGS. 2C-2E). Thus, multiple ssDNAs can be used to independently address different memory units (FIGS. 3E-3G), and genomic memory is stably recorded into DNA and can be used to modify functional genes (FIGS. 2-4). SCRIBE memory units can be decomposed into separate “Input,” “Write,” and “Read” operations to facilitate greater control and the integration of logic with memory. To demonstrate this, a synthetic gene circuit was built, which can record different input magnitudes into DNA memory, which can then be read out later upon addition of a secondary signal (after the initial input is removed). Specifically, an IPTG-inducible lacZ_{OFF} (lacZ_{A35TAA, S36TAG}) reporter construct was built in DH5αPRO cells (FIG. 8A). This reporter enables an easy population-level readout of the memory based on total LacZ activity (FIG. 8B). The lacZ_{OFF} reporter cells were transformed with a plasmid encoding an aTc-inducible SCRIBE(lacZ)_{ON} cassette (FIG. 8A). Overnight cultures were diluted and induced with various amounts of aTc (“Input & Write” signal, FIG. 8B). These cells were grown up to saturation and then diluted into fresh media in the presence or absence of IPTG (“Read” signal, FIG. 8B). In the absence of IPTG, the total LacZ activity remained low, regardless of the aTc concentration. In the presence of IPTG, cultures that had been exposed to higher aTc concentrations had greater total LacZ activity. These results show that population-level reading of genomically encoded memory can be decoupled from writing and controlled externally. Furthermore, this circuit enables the magnitude of the “Input & Write” signal (aTc) to be stably recorded in the distributed genomic memory of a cellular population. Independent control over memory operations could help to minimize fitness costs associated with the expression of reporter genes until needed.

### Example 10

The “Input” and “Write” signals can be further separated to create a synthetic sample-and-hold circuit that records information about the “Input” only when the “Write” signal is present. The separation of these signals would enable master control over the writing of multiple independent inputs into genomic memory. To achieve this, the ssDNA(lacZ)_{ON} cassette was placed under the control of an AHL-inducible promoter (P_{luxR}) (S. Basu, et al. Nature 434, 1130-1134 (2005)) and co-transformed this plasmid with an aTc-inducible Beta-expressing plasmid into the lacZ_{OFF} reporter strain (FIG. 8D). Using this design, information on the “Input” (AHL) can be written into DNA memory only in the presence of the “Write” signal (aTc). The information recorded in the memory register (e.g., the state of lacZ across the population) can be retrieved by adding the “Read” signal (IPTG). To demonstrate this, overnight lacZ_{OFF} cultures harboring the circuit shown in FIG. 8D were diluted and then grown to saturation in the presence of all four possible combinations of AHL and aTc (FIG. 8E). The saturated cultures were then diluted into fresh media in the absence or presence of IPTG. As shown in FIG. 8F, only cultures that had been exposed to both the “Input” and “Write” signals simultaneously showed significant LacZ activity, and only when they were induced with the “Read” signal. These results indicate that short stretches of DNA of living organisms can be used as addressable read/write memory registers to record transcriptional inputs. Furthermore, SCRIBE memory can be combined with logic, such as the AND function between the “Input” and “Write” signals shown here. Additional logic circuits can be combined with SCRIBE-based memory to create more complex analog-memory-and-computation systems capable of storing the results of multi-input calculations.

### Example 11

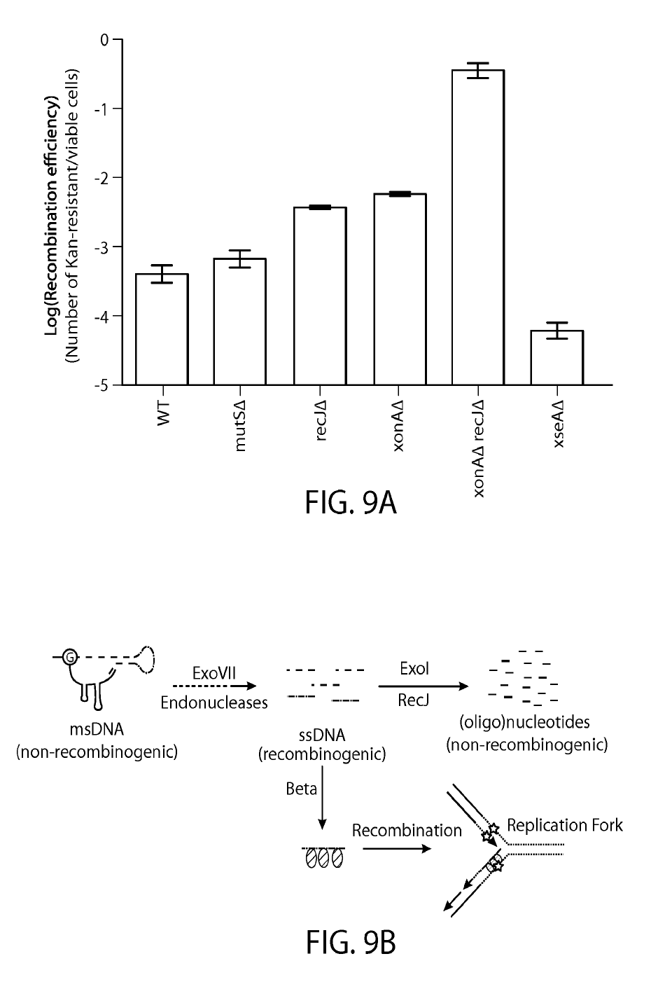

To investigate the effect of cellular factors on efficiency of SCRIBE, four candidate genes (namely mutS, recJ, xonA, and xseA) were knocked out in the reporter strain (DH5alpha PRO galK::kanR_{OFF}). As shown in FIG. 9A, strains lacking recJ and xonA (which respectively encode for exonucleases RecJ and ExoI in E. coli) showed up to 10 folds improvement in recombination efficiency. Knocking out mutS did not result in significant increase in the recombination efficiency while knocking out the xseA (which encodes one of the two subunits of Exo VII complex in E. coli) leads to reduced recombination levels. A double exonuclease mutant (xonAΔ recJΔ) was then constructed to test the synergistic effect of absence of the two exonucleases. The double exo knock out strain (DH5alpha PRO galK::kanR_{OFF} xonAΔ recJΔ) showed significant increase in recombination efficiency relative to the WT strain. In this strain, recombination efficiency up to 36% achieved (based on KanR reversion assay described earlier). This recombination efficiency is comparable to the highest recombination efficiencies reported in the literature in a mutS background to date. In order to be able to achieve high recombination efficiency only when needed and in response to a certain inducer, the recently described CRISPRi system can be leveraged to conditionally knock down recJ and xonA. Using CRISPRi, expression of these two genes can be knocked out only when higher recombination efficiency is needed and the genes turned back on when the recombination/mutation phase is over, to minimize any possible negative effect (e.g., background/unwanted mutation/recombination) that may arise in an exonuclease deficient background.

Knocking out xseA, which encodes for a third exonuclease in E. coli, reduced the efficiency of recombination in the KanR reversion assay. It has been shown that in vitro, xseA cleaves large fragments of ssDNA into small pieces. These small fragments then can be further processed into smaller pieces (and single nucleotides) by more processive exonucleases (e.g., RecJ and ExoI). The expressed ssDNA(kanR)_{ON} is flanked by the backbone of the msDNA sequence (the lower part of the msd stem). Due to presence of this flanking region, the msDNA is expected to be less recombinogenic than ssDNA sequence lacking the msd backbone. Without being bound by theory, the result provided herein suggests a model where the expressed msDNA (containing the msd backbone, less recombinogenic) is first processed by Exo VII into smaller ssDNA pieces (lacking the msd backbone, more recombinogenic) (FIG. 9B). These small pieces then can be processed (degraded) further by RecJ and ExoI into single nucleotides. This process could be a part of an endogenous pathway for metabolism of DNA.

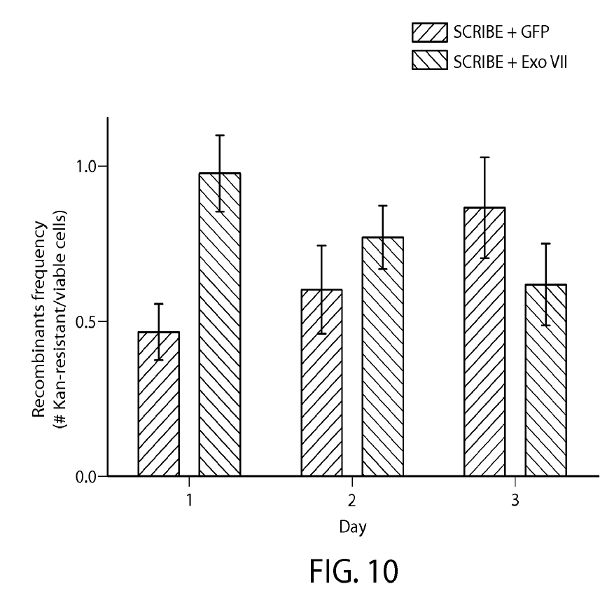

To further investigate this model, genes encoding the subunits of Exo VII of E. coli (xseA and xseB) were cloned in a synthetic operon and placed under control of aTc inducible promoter (P_{tetO}_{_}xseA_{—xseB}). Furthermore, a DH5alpha bioA::kanR_{OFF} reporter was constructed. These reporter cells were cotransformed with P_{lacO}_{_}SCRIBE(kanR)_{ON} and either of P_{tetO}_{_}xseA_xseB or P_{tetO}_{_}gfp as negative control. Single colonies were grown in LB+appropriate selection for 3 days without dilution. At the end of each day, aliquots of the samples were taken and plated on appropriate selective media to calculate the recombination efficiencies. As shown in FIG. 10, after 24 hours of induction, in cells overexpressing the SCRIBE and Exo VII complex, the frequency of the recombinants in the population reaches ˜97% which gradually declines over time, likely due to reduced competitive fitness of these cells in compare to mutants that may arise in the population. The recombination efficiency could be further optimized by conditional expression of the Exo VII complex. On the other hand, the frequency of the recombinants in the population increases significantly over time in cells expressing the SCRIBE and GFP. This suggests that prolonged incubation favors the enhanced recombination frequencies in the population.

The recombination efficiencies achieved with two strategies (prolonged incubation of cells overexpressing the SCRIBE cassette or short incubation of cells expressing SCRIBE+Exo VII complex) surpass the efficiencies achieved by the current genome engineering techniques including MAGE and its adaptation in modified hosts. The described high recombination efficiency is particularly useful, for example, for multiplexed genome engineering where multiple modifications can be introduced across a genome in one round, allowing editing multiple loci of bacterial genome at once or highly multiplexed genome engineering through iterative cycles. Alternatively the technique can be used to introduce markerless modification into bacterial genome.

### Example 12

In order to investigate whether SCRIBE's genomically-encoded memory could be read out using high-throughput sequencing, the genomic content of bacterial populations at the kanR locus were analyzed using ILLUMINA® Hi-Seq. Overnight cultures of three independent colonies harboring the gene circuit shown in FIG. 2C were diluted into fresh media and then incubated with inducers (1 mM IPTG and 100 ng/ml aTc) or without inducers for 24 hours at 30° C. As an additional control, cells expressing ssDNA(kanR)_{OFF} (which has the exact ssDNA template sequence as genomic kanR_{OFF}) were included in this experiment and grown similarly. After 24 hours of induction, total genomic DNA was prepared from the samples using Zymo ZR Fungal/Bacterial DNA MiniPrep™ Kit. Using these genomic DNA preps as template, the kanR locus was PCR-amplified by primers FF_oligo183 and FF_oligo185. After gel purification, another round of PCR was performed (using primers FF_oligo1291 and FF_oligo1292) to add ILLUMINA® adaptors as well as a 10 bp randomized nucleotide to increase the diversity of the library. Barcodes and ILLUMINA® anchors were then added using an additional round of PCR. Samples were then gel-purified, multiplexed, and run on a lane of ILLUMINA® Hi-Seq.

The obtained reads were processed and demultiplexed by the MIT BMC-BCC Pipeline. These reads were then trimmed to remove the added 10 bp randomized sequence. To filter out any reads that could have been produced by non-specific binding of primers during PCR, reads that lacked the expected “CGCGNNNNNATTT” (SEQ ID NO: 31) motif, where “NNNNN” corresponds to the 5 base-pair kanR memory register, were discarded. Furthermore, any reads that contained ambiguous bases within this 5 base-pair memory register were discarded. The frequencies of the obtained variants (either GGCCC (kanR_{ON}) or CTATT (kanR_{OFF}), which constitute the two states of the kanR memory register (FIG. 2E)), were then calculated for each sample.

As shown in Table 6, the frequency of reads mapping to kanR_{ON} in the induced samples expressing ssDNA(kanR)_{ON} was comparable to the frequency of Kan-resistant colonies obtained from the plating assay in the KanR reversion assay (FIG. 2E). Very few reads mapping to ssDNA(kanR)_{ON} were observed in the non-induced samples. Interestingly, a few reads mapping to ssDNA(kanR)_{ON} were observed in induced samples expressing ssDNA(kanR)_{OFF}. To better understand the source of these reads, the variants observed in the 5 bp kanR memory register were analyzed. These variants and their corresponding frequencies are shown for one representative sample for P_{lacO}_{_}msd(kanR)_{OFF}+P_{tetO}_{_}bet+IPTG+aTc Rep#1 in Table 7. In all the samples, less than 25 variants out of the total 1024 (4⁵=1024) possible variants were observed. Reads mapping exactly to kanR_{OFF} constituted the majority of reads, as expected. Reads with one or two base pair mutations relative to kanR_{OFF} were observed in all the samples, with frequencies ranging from 10⁻⁷-10⁻³. These reads were likely produced by the relatively high mutation rate of high-throughput sequencing or during library preparation steps. Reads with more than 2 bps of mismatch to both kanR_{ON} and kanR_{OFF} were not observed. In the negative control sample of Table 7 (in which ssDNA(KanR)_{OFF} was expressed and no kanR_{ON} sequence was present), the absence of reads with 3 or 4 mismatches to kanR_{OFF} suggests that the observed kanR_{ON} reads were likely an artifact of multiplexed sequencing, such as barcode mis-assignment or recombination during the sequencing protocol.

Overall, these results indicate that high-throughput sequencing can be used to readout genomically encoded memory. The occurrence of false-positive reads (due to sequencing errors) can be effectively avoided by having multiple mismatches (3 bps or more) between the different memory states. Furthermore, improved library preparation methods may be used to reduce the error rate of sequencing, thus enhancing readout accuracy.

TABLE 6
Frequency of reads that perfectly match to kanRON or kanROFF after
writing with SCRIBE. The sequences attributed to kanRON and kanROFF are reverse
complemented with respect to the sequences in FIG. 2D.

| | Frequency of reads that perfectly match to | | | | | | | |
|---|---|---|---|---|---|---|---|---|
| | kanR_{OFF} (CTATT) | | | | kanR_{ON} (GGCCC) | | | |
| | Rep #1 | Rep #2 | Rep #3 | Mean | Rep #1 | Rep #2 | Rep #3 | Mean |
| P_{lacO}_msd(kanR)_{ON} + | 9.98*10⁻¹ | 9.98*10⁻¹ | 9.98*10⁻¹ | 9.98*10⁻¹ | 4.35*10⁻⁴ | 4.10*10⁻⁴ | 3.87*10⁻⁴ | 4.11*10⁻⁴ |
| P_{tetO}_bet + IPTG + aTc | | | | | | | | |
| P_{lacO}_msd(kanR)_{ON} + | 9.98*10⁻¹ | 9.98*10⁻¹ | 9.98*10⁻¹ | 9.98*10⁻¹ | 0 | 8.88*10⁻⁷ | 0 | 2.96*10⁻⁷ |
| P_{tetO}_bet | | | | | | | | |
| P_{lacO}_msd(kanR)_{OFF} + | 9.98*10⁻¹ | 9.98*10⁻¹ | 9.98*10⁻¹ | 9.98*10⁻¹ | 6.26*10⁻⁷ | 0 | 3.33*10⁻⁷ | 3.20*10⁻⁷ |
| P_{tetO}_bet + IPTG + aTc | | | | | | | | |

TABLE 7
Sequencing variants and their corresponding frequencies observed in the
5 bp kanR memory register in one representative sample from cells induced
to express ssDNA(kanR)_{OFF} within a genomic kanR_{OFF} background
(P_{lacO}_msd(kanR)_{OFF} + P_{tetO}_bet + IPTG + aTc Rep#1).

| | | | | # of mismatches | # of mismatches |
|---|---|---|---|---|---|
| | Variants observed | # of reads | | relative to | relative to |
| | in the 5 bp kanR | mapped to | | kanR_{OFF} | kanR_{ON} |
| Row | memory register | the variant | Frequency | (CTATT) | (GGCCC) |
| 1 | CTATT | 11155669 | 9.98*10⁻¹ | 0 | 5 |
| 2 | CTACT | 3782 | 3.38*10⁻⁴ | 1 | 4 |
| 3 | CTATC | 1615 | 1.45*10⁻⁴ | 1 | 4 |
| 4 | GTATT | 175 | 1.57*10⁻⁵ | 1 | 4 |
| 5 | CTCTT | 113 | 1.01*10⁻⁵ | 1 | 4 |
| 6 | CGATT | 75 | 6.71*10⁻⁶ | 1 | 4 |
| 7 | ATATT | 6797 | 6.08*10⁻⁴ | 1 | 5 |
| 8 | CCATT | 2804 | 2.51*10⁻⁴ | 1 | 5 |
| 9 | CTAAT | 1289 | 1.15*10⁻⁴ | 1 | 5 |
| 10 | CTATA | 1097 | 9.82*10⁻⁵ | 1 | 5 |
| 11 | CTTTT | 508 | 4.55*10⁻⁵ | 1 | 5 |
| 12 | CAATT | 473 | 4.23*10⁻⁵ | 1 | 5 |
| 13 | CTGTT | 338 | 3.02*10⁻⁵ | 1 | 5 |
| 14 | TTATT | 336 | 3.01*10⁻⁵ | 1 | 5 |
| 15 | CTAGT | 120 | 1.07*10⁻⁵ | 1 | 5 |
| 16 | CTATG | 105 | 9.40*10⁻⁶ | 1 | 5 |
| 17 | CTACC | 11 | 9.84*10⁻⁷ | 2 | 3 |
| 18 | CAACT | 6 | 5.37*10⁻⁷ | 2 | 4 |
| 19 | ATATC | 2 | 1.79*10⁻⁷ | 2 | 4 |
| 20 | CTAAA | 4 | 3.58*10⁻⁷ | 2 | 5 |
| 21 | GGCCC | 7 | 6.26*10⁻⁷ | 5 | 0 |
| 22 | AGCCC | 107 | 9.57*10⁻⁶ | 5 | 1 |

### Materials and Methods

### Strains and Plasmids

Conventional cloning methods were used to construct the plasmids. Lists of strains and plasmids used in this study and the construction procedures are provided in Tables 1 and 2, respectively. The sequences for the synthetic parts and primers are provided in Tables 3 and 4.

TABLE 1
List of the reporter strains

| | Strain | | | |
|---|---|---|---|---|
| Name | Code | Construction method | Genotype | Used in |
| kanR_{OFF} | FFF144 | The kanR cassette was PCR | DH5αPRO | FIGS. 2A-2G |
| reporter | | amplified from the pBT3-SUC | galK::kanR_{W28TAA, A29TAG} | FIGS. 5A-5F |
| strain | | (Dualsystems Biotech) plasmid | | |
| | | using FF_oligo183 and FF_oligo184 | | |
| | | primers followed by a second round | | |
| | | of PCR with FF_oligo185 and | | |
| | | FF_oligo186 to add additional | | |
| | | sequences with homology to the | | |
| | | sequences flanking the galK locus. | | |
| | | The fragment then was integrated | | |
| | | into the galK locus of a DH5α strain | | |
| | | (with an integrated PRO cassette) by | | |
| | | recombineering. Two premature stop | | |
| | | codons then were introduced into | | |
| | | this kanR cassette using oligo- | | |
| | | mediated recombineering with | | |
| | | FF_oligo187 to make the kanR_{OFF} | | |
| | | strain. | | |
| kanR_{OFF} | FFF774 | The kanR_{OFF} cassette was PCR | DH5α | FIGS. 3E-3G |
| galK_{ON} | | amplified from FFF144 and | bioA::kanR_{W28TAA, A229TAG} + | FIGS. 4A-4B |
| reporter | | integrated into the bioA locus of | PRO plasmid | |
| strain | | DH5α. The cells were then | | |
| | | transformed with the PRO plasmid | | |
| | | (pZS4Int-LacI/TetR). | | |
| galK | FFF762 | DH5α cells transformed with the | DH5α + PRO plasmid | FIGS. 3A-3D |
| reporter | | PRO plasmid. | | |
| strain | | | | |
| lacZ_{OFF} | FFF798 | The lacZ α-fragment was introduced | DH5α lacZ_{A35TAA, S36TAG} + | FIGS. 8A-8F |
| reporter | | into the DH5α lacZ locus by | PRO plasmid | |
| strain | | recombineering using a PCR | | |
| | | fragment amplified from E. coli | | |
| | | MG1655 (using FF_oligo1069 and | | |
| | | FF_oligo1070). Two premature stop | | |
| | | codons were then introduced into the | | |
| | | lacZ ORF using oligo-mediated | | |
| | | recombineering with FF_oligo220 to | | |
| | | make the lacZ_{OFF} strain. These cells | | |
| | | were then transformed with the PRO | | |
| | | plasmid. | | |

TABLE 2
List of the plasmids

| | Plasmid | | |
|---|---|---|---|
| Name | Code | Construction method | Used in |
| P_{lacO}_msd(wt) | pFF753 | The wild-type retron Ec86 cassette was | FIG. 2B |
| | | PCR-amplified from E. coli BL21 and | |
| | | cloned downstream of the P_{lacO} promoter | |
| | | (PacI and BamHI sites) in the pZE32 | |
| | | plasmid. | |
| P_{lacO}_msd(wt)_dRT | pFF758 | This plasmid was produced by QuikChange | FIG. 2B |
| | | site-directed mutagenesis (using | |
| | | FF_oligo912 and FF_oligo913) primers to | |
| | | mutate the YADD active site of the RT to | |
| | | YAAA (D197A and D198A mutations) in | |
| | | the P_{lacO}_msd(wt) plasmid. | |
| P_{lacO}_msd(kanR)_{ON} | pFF530 | This plasmid was produced by introducing a | FIG. 2B |
| | | 79-bp fragment with homology to the kanR | FIGS. 2C-2E |
| | | ORF (template for ssDNA(kanR)_{ON}) and | FIGS. 5E-5F |
| | | flanked by EcoRI sites into the | |
| | | P_{lacO}_msd(wt) plasmid using QuikChange | |
| | | site-directed mutagenesis. | |
| P_{lacO}_msd(kanR)_{ON}_dRT | pFF749 | This plasmid was produced by QuikChange | FIGS. 2C-2E |
| | | site-directed mutagenesis (using | |
| | | FF_oligo912 and FF_oligo913) primers to | |
| | | mutate the YADD active site of the RT to | |
| | | YAAA (D197A and D198A mutations) in | |
| | | the P_{lacO}_msd(kanR)_{ON} plasmid. | |
| P_{tetO}_bet | pFF145 | This plasmid was constructed by cloning the | FIGS. 2C-2E |
| | | bet ORF from pKD46 plasmid downstream | FIGS. 5E-5F |
| | | of the P_{tetO} promoter (KpnI and BamHI sites) | FIGS. 8D-8F |
| | | in the pZA11 plasmid. | |
| P_{lacO}_SCRIBE(kanR)_{ON} | pFF745 | This plasmid was constructed by cloning bet | FIGS. 2F-2G |
| | | and its natural ribosome binding site (RBS) | FIGS. 3E-3G |
| | | downstream of the RT in the | FIGS. 4A-4B |
| | | P_{lacO}_msd(kanR)_{ON} plasmid (BamH1, MluI | |
| | | sites). 18 bp upstream of the bet start codon | |
| | | in the pKD46 plasmid was used as the bet | |
| | | RBS. | |
| P_{Dawn}_SCRIBE(kanR)_{ON} | pFF706 | This plasmid was constructed by replacing | FIGS. 5A-5D |
| (light inducible) | | the P_{lacO} promoter in SCRIBE(kanR)_{ON} with | |
| | | a PCR fragment containing the light- | |
| | | regulated cassettes (yfl/fixJ operon and cI | |
| | | and their corresponding promoters as shown | |
| | | in FIG. 4) from pDawn plasmid (Addgene | |
| | | # 43796). | |
| P_{lacO}_SCRIBE(galK)_{OFF} | pFF714 | This plasmid was constructed by replacing | FIGS. 3A-3D |
| | | the 79-bp kanR homology in | |
| | | P_{lacO}_SCRIBE(kanR)_{ON} with a 78-bp | |
| | | fragment containing two stop codons flanked | |
| | | by 72 bp homology to galK using | |
| | | QuikChange site-directed mutagenesis. | |
| P_{tetO}_SCRIBE(galK)_{OFF} | pFF761 | This plasmid was constructed by cloning the | FIGS. 3E-3G |
| | | SCRIBE(galK)_{OFF} cassette into the pZA11 | FIGS. 4A-4B |
| | | plasmid downstream of the P_{tetO} promoter | |
| | | and replacing the RBS for bet with a stronger | |
| | | RBS (RBS-A described in. | |
| P_{tetO}_SCRIBE(galK)_{ON} | pFF746 | This plasmid constructed by cloning | FIGS. 3A-3D |
| | | SCRIBE(galK)_{OFF} in the pZA21 backbone | |
| | | (downstream of P_{tetO}) followed by a | |
| | | QuikChange in vitro mutagenesis step to | |
| | | revert the two stop codons in the | |
| | | msd(galK)_{OFF} back to the wild-type | |
| | | sequence. | |
| P_{tetO}_SCRIBE(lacZ)_{ON} | pFF838 | This plasmid was made by cloning a 78-bp | FIGS. 8A-8C |
| | | fragment from the lacZ ORE into EcoRI sites | |
| | | of the SCRIBE cassette in | |
| | | P_{tetO}_SCRIBE(galK)_{ON}, replacing the galK | |
| | | homology with lacZ homology. The obtained | |
| | | SCRIBE cassette then was cloned into the | |
| | | pZA31 backbone. | |
| P_{luxR}_msd(lacZ)_{ON} | pFF828 | This plasmid was made by replacing the P_{lacO} | FIGS. 8D-8F |
| | | in the P_{lacO}_msd(kanR)_{ON} plasmid with an | |
| | | AHL-inducible promoter (luxR cassette and | |
| | | P_{luxR} promoter) followed by the replacement | |
| | | of the ssDNA(kanR)_{ON} template with a 78-bp | |
| | | fragment from the lacZ ORF. | |

TABLE 3
List of the synthetic parts and their corresponding sequences

| Part name | Type | Sequence |
|---|---|---|
| P_{lacO} | Promoter | AATTGTGAGCGGATAACAATTGACATTGTGAGCGGATAACAAGATAC |
| | | TGAGCACATCAGCAGGACGCACTGACC |
| | | (SEQ ID NO: 1) |
| P_{tetO} | Promoter | TCCCTATCAGTGATAGAGATTGACATCCCTATCAGTGATAGAGATAC |
| | | TGAGCACATCAGCAGGACGCACTGACC |
| | | (SEQ ID NO: 2) |
| P_{luxR} | Promoter | ACCTGTAGGATCGTACAGGTTTACGCAAGAAAATGGTTTGTTATAGT |
| | | CGAATA |
| | | (SEQ ID NO: 3) |
| P_{λR} | Promoter | TAACACCGTGCGTGTTGACTATTTTACCTCTGGCGGTGATAATGGTT |
| | | GC |
| | | (SEQ ID NO: 4) |
| P_{fixK2} | Promoter | ACGCCCGTGATCCTGATCACCGGCTATCCGGACGAAAACATCTCGAC |
| | | CCGGGCCGCCGAGGCCGGCGTAAAAGACGTGGTTTTGAAGCCGCTTC |
| | | TCGACGAAAACCTGCTCAAGCGTATCCGCCGCGCCATCCAGGACCGG |
| | | CCTCGGGCATGACCTACGGGGTTCTACGTAAGGCACCCCCCTTAAGA |
| | | TATCGCTCGAAATTTTCGAACCTCCCGATACCGCGTACCAATGCGTC |
| | | ATCACAACGGAG |
| | | (SEQ ID NO: 5) |
| msr | Primer for the | ATGCGCACCCTTAGCGAGAGGTTTATCATTAAGGTCAACCTCTGGAT |
| | RT | GTTGTTTCGGCATCCTGCATTGAATCTGAGTTACT |
| | | (SEQ ID NO: 6) |
| msd(wt) | Template for | GTCAGAAAAAACGGGTTTCCTGGTTGGCTCGGAGAGCATCAGGCGAT |
| | the RT | GCTCTCCGTTCCAACAAGGAAAACAGACAGTAACTCAGA |
| | | (SEQ ID NO: 7) |
| msd(kanR)_{ON} | Template for | GTCAGAAAAAACGGGTTTCCTGAATTCCAACATGGATGCTGATTTAT |
| | the RT | ATGGGTATAAATGGGCCCGCGATAATGTCGGGCAATCAGGTGCGACA |
| | | ATCTATCGGAATTCAGGAAAACAGACAGTAACTCAGA |
| | | (SEQ ID NO: 8) |
| msd(gaiK)_{OFF} | Template for | GTCAGAAAAAACGGGTTTCCTGAATTCCAGCTAATTTCCGCGCTCGG |
| | the RT | CAAGAAAGATCATGCCTAATGAATCGATTGCCGCTCACTGGGGACCA |
| | | AAGCAGTTTCCGAATTCAGGAAAACAGACAGTAACTCAGA |
| | | (SEQ ID NO: 9) |
| msd(galK)_{ON} | Template for | GTCAGAAAAAACGGGTTTCCTGAATTCCAGCTAATTTCCGCGCTCGG |
| | the RT | CAAGAAAGATCATGCCCTCTTGATCGATTGCCGCTCACTGGGGACCA |
| | | AAGCAGTTTCCGAATTCAGGAAAACAGACAGTAACTCAGA |
| | | (SEQ ID NO: 10) |
| msd(lacZ)_{ON} | Template for | GTCAGAAAAAACGGGTTTCCTGAATTCACCCAACTTAATCGCCTTGC |
| | the RT | AGCACATCCCCCTTTCGCCAGCTGGCGTAATAGCGAAGAGGCCCGCA |
| | | CCGATCGCCCTGAATTCAGGAAAACAGACAGTAACTCAGA |
| | | (SEQ ID NO: 11) |
| RT Ec86 | Reverse | ATGAAATCCGCTGAATATTTGAACACTTTTAGATTGAGAAATCTCGG |
| | Transcriptase | CCTACCTGTCATGAACAATTTGCATGACATGTCTAAGGCGACTCGCA |
| | | TATCTGTTGAAACACTTCGGTTGTTAATCTATACAGCTGATTTTCGC |
| | | TATAGGATCTACACTGTAGAAAAGAAAGGCCCAGAGAAGAGAATGAG |
| | | AACCATTTACCAACCTTCTCGAGAACTTAAAGCCTTACAAGGATGGG |
| | | TTCTACGTAACATTTTAGATAAACTGTCGTCATCTCCTTTTTCTATT |
| | | GGATTTGAAAAGCACCAATCTATTTTGAATAATGCTACCCCGCATAT |
| | | TGGGGCAAACTTTATACTGAATATTGATTTGGAGGATTTTTTCCCAA |
| | | GTTTAACTGCTAACAAAGTTTTTGGAGTGTTCCATTCTCTTGGTTAT |
| | | AATCGACTAATATCTTCAGTTTTGACAAAAATATGTTGTTATAAAAA |
| | | TCTGCTACCACAAGGTGCTCCATCATCACCTAAATTAGCTAATCTAA |
| | | TATGTTCTAAACTTGATTATCGTATTCAGGGTTATGCAGGTAGTCGG |
| | | GGCTTGATATATACGAGATATGCCGATGACCTCACCTTATCTGCACA |
| | | GTCTATGAAAAAGGTTGTTAAAGCACGTGATTTTTTATTTTCTATAA |
| | | TCCCAAGTGAAGGATTGGTTATTAACTCAAAAAAAACTTGTATTAGT |
| | | GGGCCTCGTAGTCAGAGGAAAGTTACAGGTTTAGTTATTTCACAAGA |
| | | GAAAGTTGGGATAGGTAGAGAAAAATATAAAGAAATTAGAGCAAAGA |
| | | TACATCATATATTTTGCGGTAAGTCTTCTGAGATAGAACACGTTAGG |
| | | GGATGGTTGTCATTTATTTTAAGTGTGGATTCAAAAAGCCATAGGAG |
| | | ATTAATAACTTATATTAGCAAATTAGAAAAAAAATATGGAAAGAACC |
| | | CTTTAAATAAAGCGAAGACCTAA |
| | | (SEQ ID NO: 12) |
| Beta | ssDNA- | ATGAGTACTGCACTCGCAACGCTGGCTGGGAAGCTGGCTGAACGTGT |
| | specific | CGGCATGGATTCTGTCGACCCACAGGAACTGATCACCACTCTTCGCC |
| | recombinase | AGACGGCATTTAAAGGTGATGCCAGCGATGCGCAGTTCATCGCATTA |
| | protein | CTGATCGTTGCCAACCAGTACGGCCTTAATCCGTGGACGAAAGAAAT |
| | | TTACGCCTTTCCTGATAAGCAGAATGGCATCGTTCCGGTGGTGGGCG |
| | | TTGATGGCTGGTCCCGCATCATCAATGAAAACCAGCAGTTTGATGGC |
| | | ATGGACTTTGAGCAGGACAATGAATCCTGTACATGCCGGATTTACCG |
| | | CAAGGACCGTAATCATCCGATCTGCGTTACCGAATGGATGGATGAAT |
| | | GCCGCCGCGAACCATTCAAAACTCGCGAAGGCAGAGAAATCACGGGG |
| | | CCGTGGCAGTCGCATCCCAAACGGATGTTACGTCATAAAGCCATGAT |
| | | TCAGTGTGCCCGTCTGGCCTTCGGATTTGCTGGTATCTATGACAAGG |
| | | ATGAAGCCGAGCGCATTGTCGAAAATACTGCATACACTGCAGAACGT |
| | | CAGCCGGAACGCGACATCACTCCGGTTAACGATGAAACCATGCAGGA |
| | | GATTAACACTCTGCTGATCGCCCTGGATAAAACATGGGATGACGACT |
| | | TATTGCCGCTCTGTTCCCAGATATTTCGCCGCGACATTCGTGCATCG |
| | | TCAGAACTGACACAGGCCGAAGCAGTAAAAGCTCTTGGATTCCTGAA |
| | | ACAGAAAGCCGCAGAGCAGAAGGTGGCAGCATGA |
| | | (SEQ ID NO: 13) |
| cI | λ repressor | ATGAGCACAAAAAAGAAACCATTAACACAAGAGCAGCTTGAGGACGC |
| | | ACGTCGCCTTAAAGCAATTTATGAAAAAAAGAAAAATGAACTTGGCT |
| | | TATCCCAGGAATCTGTCGCAGACAAGATGGGGATGGGGCAGTCAGGC |
| | | GTTGGTGCTTTATTTAATGGCATCAATGCATTAAATGCTTATAACGC |
| | | CGCATTGCTTGCAAAAATTCTCAAAGTTAGCGTTGAAGAATTTAGCC |
| | | CTTCAATCGCCAGAGAAATCTACGAGATGTATGAAGCGGTTAGTATG |
| | | CAGCCGTCACTTAGAAGTGAGTATGAGTACCCTGTTTTTTCTCATGT |
| | | TCAGGCAGGGATGTTCTCACCTGAGCTTAGAACCTTTACCAAAGGTG |
| | | ATGCGGAGAGATGGGTAAGCACAACCAAAAAAGCCAGTGATTCTGCA |
| | | TTCTGGCTTGAGGTTGAAGGTAATTCCATGACCGCACCAACAGGCTC |
| | | CAAGCCGAGCTTTCCTGACGGAATGTTAATTCTCGTTGACCCTGAGC |
| | | AGGCTGTTGAGCCAGGTGATTTCTGCATAGCCAGACTTGGGGGTGAT |
| | | GAGTTTACCTTCAAGAAACTGATCAGGGATAGCGGTCAGGTGTTTTT |
| | | ACAACCACTAAACCCACAGTACCCAATGATCCCATGCAATGAGAGTT |
| | | GTTCCGTTGTGGGGAAAGTTATCGCTAGTCAGTGGCCTGAAGAGACG |
| | | TTTGGCGCTGCAAACGACGAAAACTACGCTTTAGTAGCTTAA |
| | | (SEQ ID NO: 14) |
| yfl/fixJ(bicistronic | Light- | GTGGCTAGTTTTCAATCATTTGGGATACCAGGACAGCTGGAAGTCAT |
| operon) | repressible | CAAAAAAGCACTTGATCACGTGCGAGTCGGTGTGGTAATTACAGATC |
| | transcriptional | CCGCACTTGAAGATAATCCTATTGTCTACGTAAATCAAGGCTTTGTT |
| | activator | CAAATGACCGGCTACGAGACCGAGGAAATTTTAGGAAAGAACTGTCG |
| | | CTTCTTACAGGGGAAACACACAGATCCTGCAGAAGTGGACAACATCA |
| | | GAACCGCTTTACAAAATAAAGAACCGGTCACCGTTCAGATCCAAAAC |
| | | TACAAAAAAGACGGAACGATGTTCTGGAATGAATTAAATATTGATCC |
| | | AATGGAAATAGAGGATAAAACGTATTTTGTCGGTATTCAGAATGATA |
| | | TCACCGAGCACCAGCAGACCCAGGCGCGCCTCCAGGAACTGCAATCC |
| | | GAGCTCGTCCACGTCTCCAGGCTGAGCGCCATGGGCGAAATGGCGTC |
| | | CGCGCTCGCGCACGAGCTCAACCAGCCGCTGGCGGCGATCAGCAACT |
| | | ACATGAAGGGCTCGCGGCGGCTGCTTGCCGGCAGCAGTGATCCGAAC |
| | | ACACCGAAGGTCGAAAGCGCCCTGGACCGCGCCGCCGAGCAGGCGCT |
| | | GCGCGCCGGCCAGATCATCCGGCGCCTGCGCGACTTCGTTGCCCGCG |
| | | GCGAATCGGAGAAGCGGGTCGAGAGTCTCTCCAAGCTGATCGAGGAG |
| | | GCCGGCGCGCTCGGGCTTGCCGGCGCGCGCGAGCAGAACGTGCAGCT |
| | | CCGCTTCAGTCTCGATCCGGGCGCCGATCTCGTTCTCGCCGACCGGG |
| | | TGCAGATCCAGCAGGTCCTGGTCAACCTGTTCCGCAACGCGCTGGAA |
| | | GCGATGGCTCAGTCGCAGCGACGCGAGCTCGTCGTCACCAACACCCC |
| | | CGCCGCCGACGACATGATCGAGGTCGAAGTGTCCGACACCGGCAGCG |
| | | GTTTCCAGGACGACGTCATTCCGAACCTGTTTCAGACTTTCTTCACC |
| | | ACCAAGGACACCGGCATGGGCGTGGGACTGTCCATCAGCCGCTCGAT |
| | | CATCGAAGCTCACGGCGGGCGCATGTGGGCCGAGAGCAACGCATCGG |
| | | GCGGGGCGACCTTCCGCTTCACCCTCCCGGCAGCCGACGAGATGATA |
| | | GGAGGTCTAGCATGACGACCAAGGGACATATCTACGTCATCGACGAC |
| | | GACGCGGCGATGCGGGATTCGCTGAATTTCCTGCTGGATTCTGCCGG |
| | | CTTCGGCGTCACGCTGTTTGACGACGCGCAAGCCTTTCTCGACGCCC |
| | | TGCCGGGTCTCTCCTTCGGCTGCGTCGTCTCCGACGTGCGCATGCCG |
| | | GGCCTTGACGGCATCGAGCTGTTGAAGCGGATGAAGGCGCAGCAAAG |
| | | CCCCTTTCCGATCCTCATCATGACCGGTCACGGCGACGTGCCGCTCG |
| | | CGGTCGAGGCGATGAAGTTAGGGGCGGTGGACTTTCTGGAAAAGCCT |
| | | TTCGAGGACGACCGCCTCACCGCCATGATCGAATCGGCGATCCGCCA |
| | | GGCCGAGCCGGCCGCCAAGAGCGAGGCCGTCGCGCAGGATATCGCCG |
| | | CCCGCGTCGCCTCGTTGAGCCCCAGGGAGCGCCAGGTCATGGAAGGG |
| | | CTGATCGCCGGCCTTTCCAACAAGCTGATCGCCCGCGAGTACGACAT |
| | | CAGCCCGCGCACCATCGAGGTGTATCGGGCCAACGTCATGACCAAGA |
| | | TGCAGGCCAACAGCCTTTCGGAGCTGGTTCGCCTCGCGATGCGCGCC |
| | | GGCATGCTCAACGAT |
| | | (SEQ ID NO: 15) |
| kanR_{OFF} | Reporter gene | ATGAGCCATATTCAACGGGAAACGTCTTGCTCGAGGCCGCGATTAAA |
| | (premature | TTCCAACATGGATGCTGATTTATATGGGTATAAATAATAGCGCGATA |
| | stop codons | ATGTCGGGCAATCAGGTGCGACAATCTATCGATTGTATGGGAAGCCC |
| | are | GATGCGCCAGAGTTGTTTCTGAAACATGGCAAAGGTAGCGTTGCCAA |
| | underlined) | TGATGTTACAGATGAGATGGTCAGACTAAACTGGCTGACGGAATTTA |
| | | TGCCTCTTCCGACCATCAAGCATTTTATCCGTACTCCTGATGATGCA |
| | | TGGTTACTCACCACTGCGATCCCCGGGAAAACAGCATTCCAGGTATT |
| | | AGAAGAATATCCTGATTCAGGTGAAAATATTGTTGATGCGCTGGCAG |
| | | TGTTCCTGCGCCGGTTGCATTCGATTCCTGTTTGTAATTGTCCTTTT |
| | | AACAGCGATCGCGTATTTCGTCTCGCTCAGGCGCAATCACGAATGAA |
| | | TAACGGTTTGGTTGATGCGAGTGATTTTGATGACGAGCGTAATGGCT |
| | | GGCCTGTTGAACAAGTCTGGAAAGAAATGCATAAACTTTTGCCATTC |
| | | TCACCGGATTCAGTCGTCACTCATGGTGATTTCTCACTTGATAACCT |
| | | TATTTTTGACGAGGGGAAATTAATAGGTTGTATTGATGTTGGACGAG |
| | | TCGGAATCGCAGACCGATACCAGGATCTTGCCATCCTATGGAACTGC |
| | | CTCGGTGAGTTTTCTCCTTCATTACAGAAACGGCTTTTTCAAAAATA |
| | | TGGTATTGATAATCCTGATATGAATAAATTGCAGTTTCATTTGATGC |
| | | TCGATGAGTTTTTCTAA |
| | | (SEQ ID NO: 16) |
| lacZ_{OFF} | Reporter gene | ATGACCATGATTACGGATTCACTGGCCGTCGTTTTACAACGTCGTGA |
| | (premature | CTGGGAAAACCCTGGCGTTACCCAACTTAATCGCCTTGCAGCACATC |
| | stop codons | CCCCTTTCTAATAGTGGCGTAATAGCGAAGAGGCCCGCACCGATCGC |
| | are | CCTTCCCAACAGTTGCGCAGCCTGAATGGCGAATGGCGCTTTGCCTG |
| | underlined) | GTTTCCGGCACCAGAAGCGGTGCCGGAAAGCTGGCTGGAGTGCGATC |
| | | TTCCTGAGGCCGATACTGTCGTCGTCCCCTCAAACTGGCAGATGCAC |
| | | GGTTACGATGCGCCCATCTACACCAACGTGACCTATCCCATTACGGT |
| | | CAATCCGCCGTTTGTTCCCACGGAGAATCCGACGGGTTGTTACTCGC |
| | | TCACATTTAATGTTGATGAAAGCTGGCTACAGGAAGGCCAGACGCGA |
| | | ATTATTTTTGATGGCGTTAACTCGGCGTTTCATCTGTGGTGCAACGG |
| | | GCGCTGGGTCGGTTACGGCCAGGACAGTCGTTTGCCGTCTGAATTTG |
| | | ACCTGAGCGCATTTTTACGCGCCGGAGAAAACCGCCTCGCGGTGATG |
| | | GTGCTGCGCTGGAGTGACGGCAGTTATCTGGAAGATCAGGATATGTG |
| | | GCGGATGAGCGGCATTTTCCGTGACGTCTCGTTGCTGCATAAACCGA |
| | | CTACACAAATCAGCGATTTCCATGTTGCCACTCGCTTTAATGATGAT |
| | | TTCAGCCGCGCTGTACTGGAGGCTGAAGTTCAGATGTGCGGCGAGTT |
| | | GCGTGACTACCTACGGGTAACAGTTTCTTTATGGCAGGGTGAAACGC |
| | | AGGTCGCCAGCGGCACCGCGCCTTTCGGCGGTGAAATTATCGATGAG |
| | | CGTGGTGGTTATGCCGATCGCGTCACACTACGTCTGAACGTCGAAAA |
| | | CCCGAAACTGTGGAGCGCCGAAATCCCGAATCTCTATCGTGCGGTGG |
| | | TTGAACTGCACACCGCCGACGGCACGCTGATTGAAGCAGAAGCCTGC |
| | | GATGTCGGTTTCCGCGAGGTGCGGATTGAAAATGGTCTGCTGCTGCT |
| | | GAACGGCAAGCCGTTGCTGATTCGAGGCGTTAACCGTCACGAGCATC |
| | | ATCCTCTGCATGGTCAGGTCATGGATGAGCAGACGATGGTGCAGGAT |
| | | ATCCTGCTGATGAAGCAGAACAACTTTAACGCCGTGCGCTGTTCGCA |
| | | TTATCCGAACCATCCGCTGTGGTACACGCTGTGCGACCGCTACGGCC |
| | | TGTATGTGGTGGATGAAGCCAATATTGAAACCCACGGCATGGTGCCA |
| | | ATGAATCGTCTGACCGATGATCCGCGCTGGCTACCGGCGATGAGCGA |
| | | ACGCGTAACGCGAATGGTGCAGCGCGATCGTAATCACCCGAGTGTGA |
| | | TCATCTGGTCGCTGGGGAATGAATCAGGCCACGGCGCTAATCACGAC |
| | | GCGCTGTATCGCTGGATCAAATCTGTCGATCCTTCCCGCCCGGTGCA |
| | | GTATGAAGGCGGCGGAGCCGACACCACGGCCACCGATATTATTTGCC |
| | | CGATGTACGCGCGCGTGGATGAAGACCAGCCCTTCCCGGCTGTGCCG |
| | | AAATGGTCCATCAAAAAATGGCTTTCGCTACCTGGAGAGACGCGCCC |
| | | GCTGATCCTTTGCGAATACGCCCACGCGATGGGTAACAGTCTTGGCG |
| | | GTTTCGCTAAATACTGGCAGGCGTTTCGTCAGTATCCCCGTTTACAG |
| | | GGCGGCTTCGTCTGGGACTGGGTGGATCAGTCGCTGATTAAATATGA |
| | | TGAAAACGGCAACCCGTGGTCGGCTTACGGCGGTGATTTTGGCGATA |
| | | CGCCGAACGATCGCCAGTTCTGTATGAACGGTCTGGTCTTTGCCGAC |
| | | CGCACGCCGCATCCAGCGCTGACGGAAGCAAAACACCAGCAGCAGTT |
| | | TTTCCAGTTCCGTTTATCCGGGCAAACCATCGAAGTGACCAGCGAAT |
| | | ACCTGTTCCGTCATAGCGATAACGAGCTCCTGCACTGGATGGTGGCG |
| | | CTGGATGGTAAGCCGCTGGCAAGCGGTGAAGTGCCTCTGGATGTCGC |
| | | TCCACAAGGTAAACAGTTGATTGAACTGCCTGAACTACCGCAGCCGG |
| | | AGAGCGCCGGGCAACTCTGGCTCACAGTACGCGTAGTGCAACCGAAC |
| | | GCGACCGCATGGTCAGAAGCCGGGCACATCAGCGCCTGGCAGCAGTG |
| | | GCGTCTGGCGGAAAACCTCAGTGTGACGCTCCCCGCCGCGTCCCACG |
| | | CCATCCCGCATCTGACCACCAGCGAAATGGATTTTTGCATCGAGCTG |
| | | GGTAATAAGCGTTGGCAATTTAACCGCCAGTCAGGCTTTCTTTCACA |
| | | GATGTGGATTGGCGATAAAAAACAACTGCTGACGCCGCTGCGCGATC |
| | | AGTTCACCCGTGCACCGCTGGATAACGACATTGGCGTAAGTGAAGCG |
| | | ACCCGCATTGACCCTAACGCCTGGGTCGAACGCTGGAAGGCGGCGGG |
| | | CCATTACCAGGCCGAAGCAGCGTTGTTGCAGTGCACGGCAGATACAC |
| | | TTGCTGATGCGGTGCTGATTACGACCGCTCACGCGTGGCAGCATCAG |
| | | GGGAAAACCTTATTTATCAGCCGGAAAACCTACCGGATTGATGGTAG |
| | | TGGTCAAATGGCGATTACCGTTGATGTTGAAGTGGCGAGCGATACAC |
| | | CGCATCCGGCGCGGATTGGCCTGAACTGCCAGCTGGCGCAGGTAGCA |
| | | GAGCGGGTAAACTGGCTCGGATTAGGGCCGCAAGAAAACTATCCCGA |
| | | CCGCCTTACTGCCGCCTGTTTTGACCGCTGGGATCTGCCATTGTCAG |
| | | ACATGTATACCCCGTACGTCTTCCCGAGCGAAAACGGTCTGCGCTGC |
| | | GGGACGCGCGAATTGAATTATGGCCCACACCAGTGGCGCGGCGACTT |
| | | CCAGTTCAACATCAGCCGCTACAGTCAACAGCAACTGATGGAAACCA |
| | | GCCATCGCCATCTGCTGCACGCGGAAGAAGGCACATGGCTGAATATC |
| | | GACGGTTTCCATATGGGGATTGGTGGCGACGACTCCTGGAGCCCGTC |
| | | AGTATCGGCGGAATTCCAGCTGAGCGCCGGTCGCTACCATTACCAGT |
| | | TGGTCTGGTGTCAAAAATAA |
| | | (SEQ ID NO: 17) |
| SCRIBE(kanR)_{ON} | The synthetic | ATGCGCACCCTTAGCGAGAGGTTTATCATTAAGGTCAACCTCTGGAT |
| | operon for | GTTGTTTCGGCATCCTGCATTGAATCTGAGTTACTGTCTGTTTTCCT |
| | writing into | GAATTCCGATAGATTGTCGCACCTGATTGCCCGACATTATCGCGGGC |
| | the kanR | CCATTTATACCCATATAAATCAGCATCCATGTTGGAATTCAGGAAAC |
| | locus. | CCGTTTTTTCTGACGTAAGGGTGCGCAACTTTCATGAAATCCGCTGA |
| | The | ATATTTGAACACTTTTAGATTGAGAAATCTCGGCCTACCTGTCATGA |
| | msd(kanR)_{ON} | ACAATTTGCATGACATGTCTAAGGCGACTCGCATATCTGTTGAAACA |
| | region is | CTTCGGTTGTTAATCTATACAGCTGATTTTCGCTATAGGATCTACAC |
| | underlined. | TGTAGAAAAGAAAGGCCCAGAGAAGAGAATGAGAACCATTTACCAAC |
| | The region | CTTCTCGAGAACTTAAAGCCTTACAAGGATGGGTTCTACGTAACATT |
| | flanked by | TTAGATAAACTGTCGTCATCTCCTTTTTCTATTGGATTTGAAAAGCA |
| | EcoRI sites | CCAATCTATTTTGAATAATGCTACCCCGCATATTGGGGCAAACTTTA |
| | (red) can be | TACTGAATATTGATTTGGAGGATTTTTTCCCAAGTTTAACTGCTAAC |
| | replaced with | AAAGTTTTTGGAGTGTTCCATTCTCTTGGTTATAATCGACTAATATC |
| | a template for | TTCAGTTTTGACAAAAATATGTTGTTATAAAAATCTGCTACCACAAG |
| | ssDNAs of | GTGCTCCATCATCACCTAAATTAGCTAATCTAATATGTTCTAAACTT |
| | interest. | GATTATCGTATTCAGGGTTATGCAGGTAGTCGGGGCTTGATATATAC |
| | | GAGATATGCCGATGACCTCACCTTATCTGCACAGTCTATGAAAAAGG |
| | | TTGTTAAAGCACGTGATTTTTTATTTTCTATAATCCCAAGTGAAGGA |
| | | TTGGTTATTAACTCAAAAAAAACTTGTATTAGTGGGCCTCGTAGTCA |
| | | GAGGAAAGTTACAGGTTTAGTTATTTCACAAGAGAAAGTTGGGATAG |
| | | GTAGAGAAAAATATAAAGAAATTAGAGCAAAGATACATCATATATTT |
| | | TGCGGTAAGTCTTCTGAGATAGAACACGTTAGGGGATGGTTGTCATT |
| | | TATTTTAAGTGTGGATTCAAAAAGCCATAGGAGATTAATAACTTATA |
| | | TTAGCAAATTAGAAAAAAAATATGGAAAGAACCCTTTAAATAAAGCG |
| | | AAGACCTAAGGATCCGGTTGATATTATTCAGAGGTATAAAACGAATG |
| | | AGTACTGCACTCGCAACGCTGGCTGGGAAGCTGGCTGAACGTGTCGG |
| | | CATGGATTCTGTCGACCCACAGGAACTGATCACCACTCTTCGCCAGA |
| | | CGGCATTTAAAGGTGATGCCAGCGATGCGCAGTTCATCGCATTACTG |
| | | ATCGTTGCCAACCAGTACGGCCTTAATCCGTGGACGAAAGAAATTTA |
| | | CGCCTTTCCTGATAAGCAGAATGGCATCGTTCCGGTGGTGGGCGTTG |
| | | ATGGCTGGTCCCGCATCATCAATGAAAACCAGCAGTTTGATGGCATG |
| | | GACTTTGAGCAGGACAATGAATCCTGTACATGCCGGATTTACCGCAA |
| | | GGACCGTAATCATCCGATCTGCGTTACCGAATGGATGGATGAATGCC |
| | | GCCGCGAACCATTCAAAACTCGCGAAGGCAGAGAAATCACGGGGCCG |
| | | TGGCAGTCGCATCCCAAACGGATGTTACGTCATAAAGCCATGATTCA |
| | | GTGTGCCCGTCTGGCCTTCGGATTTGCTGGTATCTATGACAAGGATG |
| | | AAGCCGAGCGCATTGTCGAAAATACTGCATACACTGCAGAACGTCAG |
| | | CCGGAACGCGACATCACTCCGGTTAACGATGAAACCATGCAGGAGAT |
| | | TAACACTCTGCTGATCGCCCTGGATAAAACATGGGATGACGACTTAT |
| | | TGCCGCTCTGTTCCCAGATATTTCGCCGCGACATTCGTGCATCGTCA |
| | | GAACTGACACAGGCCGAAGCAGTAAAAGCTCTTGGATTCCTGAAACA |
| | | GAAAGCCGCAGAGCAGAAGGTGGCAGCATGA |
| | | (SEQ ID NO: 18) |

TABLE 4
List of the synthetic oligonucleotides (oligos)

| Name | Sequence |
|---|---|
| FF_oligo183 | GCGATATCCATTTTCGCGAATCCGGAGTGTAAGAAGAGCTCCTGACTCCCCGTCGTGTAG |
| | (SEQ ID NO: 19) |
| FF_oligo184 | GACCGCAGAACAGGCAGCAGAGCGTTTGCGCGCAGTCAGCGATATCCATTTTCGCGAATC |
| | (SEQ ID NO: 20) |
| FF_oligo185 | CGGCTGACCATCGGGTGCCAGTGCGGGAGTTTCGTGACGTCGTTAAGCCAGCCCCGACAC |
| | (SEQ ID NO: 21) |
| FF_oligo186 | ACTACCATCCCTGCGTTGTTACGCAAAGTTAACAGTCGGTACGGCTGACCATCGGGTGCC |
| | (SEQ ID NO: 22) |
| FF_oligo187 | C*G*CGATTAAATTCCAACATGGATGCTGATTTATATGGGTATAAATAATAGCGCGATAA |
| (* shows | TGTCGGGCAATCAGGTGCGACAATCTATCG*A*T |
| phosphorothioate | (SEQ ID NO: 23) |
| bond) | |
| FF_oligo220 | CAACTTAATCGCCTTGCAGCACATCCCCCTTTCTAATAGTGGCGTAATAGCGAAGAGGCC |
| | CGCACCGATCGC |
| | (SEQ ID NO: 24) |
| FF_oligo912 | GATATATACGAGATATGCCGCTGCTCTCACCTTATCTGCAC |
| | (SEQ ID NO: 25) |
| FF_oligo913 | GTGCAGATAAGGTGAGAGCAGCGGCATATCTCGTATATATC |
| | (SEQ ID NO: 26) |
| FF_oligo1069 | AATACGCAAACCGCCTCTCC |
| | (SEQ ID NO: 27) |
| FF_oligo1070 | CGGCGGATTGACCGTAATGG |
| | (SEQ ID NO: 28) |
| FF_oligo347 | GTCAGAAAAAACGGGTTTCCTGGTTGGCTCGGAGAGCATCAGGCGATGCTCTCCGTTCCA |
| (PAGE purified, | ACAAGGAAAACAGACAGTAACTCAGA |
| used as ssDNA | (SEQ ID NO: 29) |
| size marker in | |
| FIG. 2B) | |

### Cells and Antibiotics

Chemically competent E. coli DH5α was used for cloning. Unless otherwise noted, antibiotics were used at the following concentrations to maintain plasmids in liquid cultures: carbenicillin (50 μg/ml), kanamycin (20 μg/ml), chloramphenicol (30 μg/ml) and spectinomycin (100 μg/ml).

### Experimental Procedure

ssDNA Detection

Total RNA samples were prepared from non-induced or induced cells using TRIzol reagent (Invitrogen) according to the manufacturer's protocol. 10 μg total RNA from each sample was treated with RNase A (1 μl, 37° C., 2 hours) to remove RNA species and the msr moiety. The samples were then resolved on 10% TBE-Urea denaturing gel and visualized with SYBR-Gold. A PAGE-purified synthetic oligo (FF_oligo347, Integrated DNA Technologies) with the same sequence as ssDNA(wt) was used as a molecular size marker.

### Induction of Cells and Plating Assays

For each experiment, three transformants were separately inoculated in LB media+appropriate antibiotics and grown overnight (37° C., 700 RPM) to obtain seed cultures. Unless otherwise noted, inductions were performed by diluting the seed cultures (1:1000) in 2 ml of pre-warmed LB+appropriate antibiotics±inducers followed by 24 hours incubation (30° C., 700 RPM). Aliquots of the samples were then serially diluted and appropriate dilutions were plated on selective media to determine the number of recombinants and viable cells in each culture. For each sample, the recombinant frequency was reported as the mean of the ratio of recombinants to viable cells for three independent replicates.

In all the experiments, the number of viable cells was determined by plating aliquots of cultures on LB+spectinomycin plates. LB+kanamycin plates were used to determine the number of recombinants in the kanR reversion assay. For the galK reversion assay (FIGS. 3A-3D), the numbers of galK_{ON} recombinants were determined by plating the cells on MOPS EZ rich defined media (Teknova)+galactose (0.2%). The numbers of galK_{OFF} recombinants were determined by plating the cells on MOPS EZ rich defined media+glycerol (0.2%)+2-DOG (2%). For the experiment shown in FIGS. 3E-3G, the numbers of kanR_{ON} galK_{ON} and kanR_{OFF} galK_{OFF} cells were determined by using LB+kanamycin plates and MOPS EZ rich defined media+glycerol (0.2%)+2-DOG (2%)+D-biotin (0.01%), respectively. The numbers of kanR_{ON} galK_{OFF} cells in FIGS. 4A and 4B were determined by plating the cells on MOPS EZ rich defined media+glycerol (0.2%)+2-DOG (2%)+kanamycin+D-biotin (0.01%). For the light-inducible SCRIBE experiment (FIGS. 5A-5D), induction was performed with white light (using the built-in fluorescent lamp in a VWR 1585 shaker incubator). The “dark” condition was achieved by wrapping aluminum foil around the tubes. Growth of these cultures and sampling from these cultures were performed as described earlier.

### LacZ Assay

Overnight seed cultures were diluted (1:1000) in pre-warmed LB+appropriate antibiotics and inducers (with different concentrations of aTc or without aTc in FIGS. 8A-8C, and with all the four possible combinations of aTc and AHL in FIGS. 8D-8F) and incubated for 24 hours (30 C, 700 RPM). These cultures then were diluted (1:50) in pre-warmed LB+appropriate antibiotics with or without IPTG and incubated for 8 hours (37° C., 700 RPM). To measure LacZ activity, 60 μl of each culture was mixed with 60 μl of B-PER II reagent (Pierce Biotechnology) and Fluorescein Di-β-D-Galactopyranoside (FDG, 0.05 mg/ml final concentration). The fluorescence signal (absorption/emission: 485/515) was monitored in a plate reader with continuous shaking for 2 hours. The LacZ activity was calculated by normalizing the rate of FDG hydrolysis (obtained from fluorescence signal) to the initial OD. For each sample, LacZ activity was reported as the mean of three independent biological replicates.

### Modeling and Simulation

### Deterministic Model

The accumulation of recombinants was modeled in growing cell populations. The model assumes that clonal interference is negligible, and that the recombinant and wild-type alleles are equally fit. In other words, the model assumes that all the cells in the population have the same growth profile. It also assumes that the rate of recombination in the reverse direction (e.g., from the genome to the plasmid) is negligible (the rate of recombination in recA-background is <10⁻¹⁰ (S. T. Lovett, et al. Genetics 160, 851-859 (2002)). The model also assumes that after each Beta-mediated recombination event, only one of the two daughter cells becomes recombinant (M. S. Huen, et al. Nucleic Acids Res 34, 6183-6194 (2006); K. C. Murphy, et al. F1000 Biol Rep 2, 56 (2010)).

For a given time (t), the recombinant frequency (fₜ) is defined as the ratio between the number of recombinants (mₜ) to the total number of viable cells in the population (NO.

$${f}_{t} = \frac{{m}_{t}}{{N}_{t}}$$

The recombination rate (r) represents the frequency of recombination events that happen in one generation (dt). After one generation, the number of viable cells doubles (N_{t+dt} =2Nₜ). The number of recombinants in the culture is the sum of the number of cells that are progeny of pre-existing recombinants and new recombinants that are produced during that generation (m_{t+dt}=2mₜ+(Nₜ−mₜ)r). Thus:

$${f}_{t + dt} = \frac{2 {m}_{t} + ( {N}_{t} - {m}_{t} ) r}{2 {N}_{t}} = {f}_{t} + \frac{( 1 - {f}_{t} ) r}{2} ⇒ \\ {f}_{t + dt} - {f}_{t} = \frac{( 1 - {f}_{t} ) r}{2} ⇒ df = \frac{( 1 - {f}_{t} ) r}{2} dt ⇒ \\ \frac{df}{1 - {f}_{t}} = \frac{r}{2} dt ⇒ \\ {f}_{t} = 1 - ( 1 - {f}_{0} ) {e}^{- \frac{r}{2} t} \\ where dt = one generation \qquad ( 1 )$$

Similarly, for two constitutive generations (t and t+1) we can write:

$${f}_{t + 1} - {f}_{t} = ( 1 - ( 1 - {f}_{0} ) {e}^{- \frac{r}{2} ( t + 1 )} ) - ( 1 - ( 1 - {f}_{0} ) {e}^{- \frac{r}{2} ( t )} ) = ( 1 - {f}_{0} ) ( {e}^{- \frac{r}{2} t} - {e}^{- \frac{r}{2} ( t + 1 )} )$$

$${f}_{t + 1} - {f}_{t} = ( 1 - {f}_{0} ) {e}^{- \frac{r}{2} t} ( 1 - {e}^{- \frac{r}{2}} ) = ( 1 - {f}_{t} ) ( 1 - {e}^{- \frac{r}{2}} ) ⇒ \\ {f}_{t + 1} = {f}_{t} + ( 1 - {f}_{t} ) ( 1 - {e}^{- \frac{r}{2}} ) = 1 - ( 1 - {f}_{t} ) {e}^{- \frac{r}{2}}$$

Equation (1) describes the frequency of recombinants in a growing bacterial population. In this equation, if

$$( \frac{r}{2} t )$$

is very small:

$${e}^{- \frac{r}{2} t} ≅ 1 - \frac{r}{2} t$$

$${f}_{t} ≅ 1 - ( 1 - {f}_{0} ) ( 1 - \frac{r}{2} t ) = \frac{r}{2} t + {f}_{0} - \frac{r}{2} {tf}_{0}$$

And if f₀ is also very small, the last term is negligible, thus yielding:

$${f}_{t} ≅ \frac{r}{2} t + {f}_{0} \qquad ( 2 )$$

Equation (2) shows that when the initial frequency of recombinants (f₀) and the recombination rate (r) are very small, the recombinant frequency in the population increases linearly over time (as long as

$$\frac{r}{2} {tf}_{0}$$

is relatively small) with a slope that is equal to half of the recombination rate. However, when those two quantities are relatively high or as the number of generations increases, the recombinant frequency will start to saturate and deviate from a straight line due to a significant drop in the number of cells that can be recombined (i.e. wild-type cells). Nonetheless, Equation (1) should still describe the accumulation of recombinants in the population.

Overall, the model predicts a linear increase (with a

$$slope = \frac{r}{2} )$$

in the recombinant frequency as long as the cells in the population are equally fit and as long as

$$\frac{r}{2} {tf}_{0}$$

is relatively small. However, mutations can occur within populations over time, which can affect the fitness of individual cells. In the absence of recombination in asexual populations, two beneficial mutations that arise independently cannot be combined into a single, superior genotype (C. A. Fogle, et al. Genetics 180, 2163-2173 (2008); M. Imhof, et al. Proc Natl Acad Sci USA 98, 1113-1117 (2001)). Hence, these carriers could compete with each other, a phenomenon known as clonal interference that is important in shaping the evolutionary trajectory of large asexual populations with high mutation rates over prolonged growth. Under these circumstances, the model assumption that all the cells in the population are equally fit does not hold and deviation from the model is expected. However, since the natural rate of beneficial mutations is low (˜10⁻⁹ per bp per generation for E. coli (M. Imhof, et al., 2001), the probability of mutations with significant fitness effects and clonal interference is relatively low, at least over the timescales of our experiments. Similarly, a linear increase in mutant frequencies during exponential growth of a bacterial culture was previously predicted (P. L. Foster, et al. Methods Enzymol 409, 195-213 (2006); S. E. Luria, Cold Spring Harb Symp Quant Biol 16, 463-470 (1951)).

### Stochastic Simulation

To further validate the model, stochastic simulations of a growing bacterial population were performed with three different recombination rates (r=10⁻⁹, 0.00015, or 0.005 events/generation) for 250 generations (FIGS. 7A-7B). The simulation started with a clonal population of bacteria (10⁶ cells). Growth was simulated for 25 iterations, with 10 generations in each iteration. During each generation, each cell could stochastically produce a recombinant allele with a likelihood equal to the recombination rate. The wild-type and recombinant cells were assumed to be equally fit. It was also assumed that all the cells in the population followed the same growth profile (no clonal interference). After 10 generations, a sample of ˜10⁶ cells was taken from the population to start a new culture in order to simulate the serial batch culture procedure.

As shown in FIG. 7A, the model predicts a linear increase in the frequency of recombinants with a very low mutation rate (r=10⁻⁹). However, the simulation results were not consistent with the deterministic model; instead, the simulation showed stochastic fluctuations in the recombinant frequency since samples taken after 10 generations may not contain representative numbers of recombinants due to the low recombination rate. This condition is representative of the recombinant frequencies observed in the absence of SCRIBE. Major recombination pathways in E. coli are recA-dependent and knocking out RecA activity can severely affect the recombination rate (B. E. Dutra, et al. Proc Natl Acad Sci USA 104, 216-221 (2007); S. T. Lovett, et al. Genetics 160, 851-859 (2002). In a recombination-deficient background (recA⁻), such as DH5α, recombination is a very rare, stochastic event (<10⁻¹⁰ events/generation). These data are consistent with FIG. 5F, where no significant increase in recombinant frequencies was observed in the absence of SCRIBE activation.

In contrast, at a higher targeted recombination rate (r=0.00015), a linear increase in the frequency of recombinants is predicted by both the model and simulation (FIG. 7B). This rate is representative of cells containing a specific locus targeted by SCRIBE memory. SCRIBE enables control over the recombination rate at a specific locus by external inputs, thus increasing the recombination rate by multiple orders of magnitude over the background rate. For example, using data shown in FIG. 5F for cells induced with both aTc and IPTG (induction pattern II), r=0.00015 events/generation was calculated based on the linear regression of the recombination frequency versus generation (FIG. 6). This recombination rate ensures that samples taken from an induced culture contain a representative number of recombinant cells. Thus, successive sampling and regrowth of cells results in the gradual accumulation of recombinants in the population over time in the presence of the inputs (FIG. 7B and FIGS. 5E-5F).

Finally, as the recombination rate increases (r=0.005, FIG. 7C), the model and simulation predict a linear increase in the recombination frequency at initial times. However, they both started to deviate from the linear approximation as the frequency of recombinants increases (above ˜5%) since the cultures are increasingly depleted of the wild-type alleles.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered nucleic acid construct, comprising:
a promoter operably linked to a nucleic acid that comprises
(a) a nucleotide sequence encoding a single-stranded msr RNA,
(b) a nucleotide sequence encoding a single-stranded msd DNA modified to contain a targeting sequence, and
(c) a nucleotide sequence encoding a reverse transcriptase protein,
wherein (a) and (b) are flanked by inverted repeat sequences.

2. The engineered nucleic acid construct of claim 1, wherein the promoter is an inducible promoter.

3. The engineered nucleic acid construct of claim 1 or 2, wherein the nucleotide sequence of (a) is upstream of the nucleotide sequence of (b), which is upstream of the nucleotide sequence of (c).

4. The engineered nucleic acid construct of any one of claims 1-3, wherein the nucleic acid further comprises a nucleotide sequence that encodes a single-stranded DNA (ssDNA)-annealing recombinase protein.

5. The engineered nucleic acid construct of claim 4, wherein the ssDNA-annealing recombinase protein is a Beta recombinase protein or a Beta recombinase protein homolog.

6. The engineered nucleic acid construct of claim 5, wherein the ssDNA-annealing recombinase protein is a bacteriophage lambda Beta recombinase protein or a bacteriophage lambda Beta recombinase protein homolog.

7. The engineered nucleic acid construct of any one of claims 4-6, wherein the nucleotide sequence that encodes a ssDNA-annealing recombinase protein is downstream relative to the nucleotide sequence of (c).

8. A cell, comprising:
at least one of the engineered nucleic acid constructs of any one of claims 1-7.

9. The cell of claim 8, comprising at least two of the engineered nucleic acid constructs.

10. The cell of claim 9, wherein at least two of the promoters are different from each other.

11. The cell of claim 9 or 10, comprising at least three of the engineered nucleic acid constructs.

12. A cell, comprising:
(a) at least one of the engineered nucleic acid constructs of any one of claims 1-3; and
(b) a single-stranded DNA (ssDNA)-annealing recombinase protein.

13. The cell of claim 12, wherein the ssDNA-annealing recombinase protein is a Beta recombinase protein or a Beta recombinase protein homolog.

14. The cell of claim 12 or 13, comprising at least two of the engineered nucleic acid constructs.

15. The cell of claim 14, wherein at least two of the promoters are different from each other.

16. The cell of claim 14 or 15, comprising at least three of the engineered nucleic acid constructs.

17. The cell of any one of claims 12-16, wherein the cell comprises an engineered nucleic acid construct comprising a promoter operably linked to a nucleic acid encoding the ssDNA-annealing recombinase protein.

18. The cell of claim 17, wherein the promoter operably linked to a nucleic acid encoding the ssDNA-annealing recombinase protein is an inducible promoter.

19. The cell of any one of claims 8-18, wherein the cell recombinantly expresses an Escherichia coli bacterial cell gene encoding XseA and/or XseB.

20. The cell of any one of claims 8-19, wherein the cell is an Escherichia coli bacterial cell that contains a deletion of a gene encoding ExoI and/or RecJ.

21. A method, comprising:
delivering to cells at least one of the engineered nucleic acid constructs of any one of claims 1-7, wherein the cell comprises a nucleotide sequence that is complementary to the targeting sequence.

22. The method of claim 21, wherein the nucleotide sequence that is complementary to the targeting sequence is a genomic DNA sequence.

23. A method, comprising:
delivering to cells (a) at least one of the engineered nucleic acid constructs of any one of claims 1-3, and (b) an engineered nucleic acid construct comprising a promoter operably linked to a nucleic acid encoding a single-stranded DNA (ssDNA)-annealing recombinase protein, wherein the cell comprises a nucleotide sequence that is complementary to the targeting sequence.

24. The method of claim 23, wherein the ssDNA-annealing recombinase protein is a Beta recombinase protein or a Beta recombinase protein homolog.

25. The method of claim 23 or 24, wherein the promoter operably linked to a nucleic acid encoding a ssDNA-annealing recombinase protein is an inducible promoter.

26. The method of any one of claims 23-25, wherein the nucleotide sequence that is complementary to the targeting sequence is a genomic DNA sequence.

27. The method of any one of claims 23-26, wherein at least two of the promoters are different from each other.

28. The method of any one of claims 21-27, further comprising exposing the cells to at least one signal that regulates transcription of at least one of the nucleic acids.

29. The method of claim 28, wherein the at least one signal activates transcription of at least one of the nucleic acids.

30. The method of claim 28 or 29, comprising exposing the cells at least twice to at least one signal that regulates transcription of at least one of the nucleic acids.

31. The method of claim 30, comprising exposing the cells at least twice over the course of at least 2 days to at least one signal that activates transcription of at least one of the nucleic acids.

32. The method of any one of claims 28-31, wherein the signal is a chemical signal or a non-chemical signal.

33. The method of claim 32, wherein the signal is a non-chemical signal, and the non-chemical signal is light.

34. The method of any one of claims 28-33, wherein the signal is an endogenous signal.

35. The method of any one of claims 28-34, further comprising calculating a recombination rate between the targeting sequence of the at least one engineered nucleic acid construct and a nucleotide sequence complementary to the targeting sequence.

36. A cell comprising:
(a) a first engineered nucleic acid construct that comprises a first promoter operably linked to a first nucleic acid that comprises
(i) a nucleotide sequence encoding a single-stranded msr RNA, and
(ii) a nucleotide sequence encoding a single-stranded msd DNA modified to contain a targeting sequence, wherein (i) and (ii) are flanked by inverted repeat sequences; and
(b) a second engineered nucleic acid construct that comprises a second promoter operably linked to a second nucleic acid that comprises a nucleotide sequence encoding a reverse transcriptase protein.

37. The cell of claim 36, wherein the first and/or second promoter is an inducible promoter.

38. The cell of claim 36 or 37, wherein the nucleotide sequence of (i) is upstream of the nucleotide sequence of (ii).

39. The cell of any one of claims 36-38, wherein the first or second nucleic acid further comprises a nucleotide sequence that encodes a single-stranded DNA (ssDNA)-annealing recombinase protein.

40. The cell of claim 39, wherein the ssDNA-annealing recombinase protein is a Beta recombinase protein or a Beta recombinase protein homolog.

41. The cell of claim 40, wherein the ssDNA-annealing recombinase protein is a bacteriophage lambda Beta recombinase protein or a bacteriophage lambda Beta recombinase protein homolog.

42. A method, comprising delivering to cells:
(a) a first engineered nucleic acid construct comprising a first inducible promoter operably linked to a first nucleic acid that comprises
(i) a nucleotide sequence encoding a single-stranded msr RNA,
(ii) a nucleotide sequence encoding a first single-stranded msd DNA modified to contain a first targeting sequence, and
(iii) optionally a nucleotide sequence encoding a reverse transcriptase protein, wherein (i) and (ii) are flanked by inverted repeat sequences; and
(b) a second engineered nucleic acid construct comprising a second inducible promoter operably linked to a second nucleic acid that comprises
(iv) a nucleotide sequence encoding a single-stranded msr RNA,
(v) a nucleotide sequence encoding a second single-stranded msd DNA modified to contain a second targeting sequence, and
(vi) a optionally nucleotide sequence encoding a reverse transcriptase protein, wherein (iv) and (v) are flanked by inverted repeat sequences.

43. The method of claim 42, wherein the first and/or second nucleic acid comprises the nucleotide sequence encoding a reverse transcriptase protein.

44. The method of claim 42, wherein the first and/or second nucleic acid does not comprises the nucleotide sequence encoding a reverse transcriptase protein, and the method further comprises delivering to the cells a third engineered nucleic acid construct comprising a promoter operably linked to a third nucleic acid that comprises a nucleotide sequence encoding a reverse transcriptase protein.

45. The method of claim 42, wherein the nucleotide sequence of (i) is upstream of the nucleotide sequence of (ii), which is upstream of the nucleotide sequence of (iii), and/or the nucleotide sequence of (iv) is upstream of the nucleotide sequence of (v), which is upstream of the nucleotide sequence of (vi).

46. The method of claim 42 or 45, wherein the method further comprises delivering to the cells an engineered nucleic acid construct that comprises a promoter operably linked to a nucleic acid encoding a single-stranded DNA (ssDNA)-annealing recombinase protein.

47. The method of claim 46, wherein the ssDNA-annealing recombinase protein is a Beta recombinase protein or a Beta recombinase protein homolog.

48. The method of claim 42 or 45, wherein the first nucleic acid and/or the second nucleic acid further comprises a nucleotide sequence encoding a ssDNA-annealing recombinase protein.

49. The method of claim 48, wherein the ssDNA-annealing recombinase protein is a Beta recombinase protein or a Beta recombinase protein homolog.

50. The method of claim 48 or 49, wherein (i) is upstream of (ii), which is upstream of (iii), which is upstream of the nucleotide sequence encoding a ssDNA-annealing recombinase protein and/or (iv) is upstream of (v), which is upstream of (vi), which is upstream of the nucleotide sequence encoding a ssDNA-annealing recombinase protein.

51. The method of any one of claims 42-50, further comprising exposing the cells to a first signal that regulates transcription of the first nucleic acid and a second signal that regulates transcription of the second nucleic acid.

52. The method of claim 51, wherein the cells are exposed to the first signal under conditions that permit recombination of the first targeting sequence of the first single-stranded msd DNA and a nucleotide sequence complementary to the first targeting sequence, and then the cells are exposed to the second signal under conditions that permit recombination of the second targeting sequence of the second single-stranded msd DNA and a nucleotide sequence complementary to the second targeting sequence.

53. The method of claim 51 or 52, wherein the exposing step is repeated at least once.

54. The method of claim 53, wherein the exposing step is repeated at least once over the course of at least 2 days.

55. The method of any one of claims 51-54, wherein the first signal and/or the second signal is a chemical signal or a non-chemical signal.

56. The method of claim 55, wherein the first signal and/or second signal is a non-chemical signal, and the non-chemical signal is light.

57. The method of any one of claims 51-56, wherein the first signal and/or second signal is an endogenous signal.

58. The method of any one of claims 42-57, wherein the first targeting sequence is complementary to a nucleotide sequence located in the genome of the cell, and the second targeting sequence is complementary to the first targeting sequence.

59. The method of any one of claims 42-57, wherein the first targeting sequence is complementary to a nucleotide sequence located in the genome of the cell, and the second targeting sequence is complementary to a nucleotide sequence located in the genome of the cell.

60. The method of claim 59, wherein the first targeting sequence is different from the second targeting nucleotide sequence.

61. The method of any one of claims 45-60, further comprising calculating a recombination rate between the first targeting sequence and a nucleotide sequence complementary to the first targeting sequence and/or calculating a recombination rate between the second targeting sequence and a nucleotide sequence complementary to the second targeting sequence.

62. A cell, comprising:
(a) a first engineered nucleic acid construct comprising a first inducible promoter operably linked to a first nucleic acid encoding a reporter protein containing at least one genetic element that prevents transcription of the reporter protein; and
(b) a second engineered nucleic acid construct comprising a second inducible promoter operably linked to a second nucleic acid that comprises
(i) a nucleotide sequence encoding a single-stranded msr RNA,
(ii) a nucleotide sequence encoding a single-stranded msd DNA modified to contain a targeting sequence complementary to the at least one genetic element that prevents transcription of the reporter protein, and
(iii) optionally a nucleotide sequence encoding a reverse transcriptase protein, wherein (i) and (ii) are flanked by inverted repeat sequences.

63. The cell of claim 62, wherein the nucleotide sequence of (i) is upstream of the nucleotide sequence of (ii), which is upstream of the nucleotide sequence of (iii).

64. The cell of claim 62 or 63, wherein the cell further comprises an engineered nucleic acid construct that comprises a promoter operably linked to a nucleic acid encoding a Beta recombinase protein or a Beta recombinase protein homolog.

65. The cell of claim 62 or 63, wherein the second nucleic acid further comprises a nucleotide sequence encoding a single-stranded DNA (ssDNA)-annealing recombinase protein.

66. The cell of claim 65, wherein the ssDNA-annealing recombinase protein is a Beta recombinase protein or a Beta recombinase protein homolog.

67. The cell of claim 65 or 66, wherein the nucleotide sequence of (i) is upstream of the nucleotide sequence of (ii), which is upstream of the nucleotide sequence of (iii), which is upstream of the nucleotide sequence encoding a ssDNA-annealing recombinase protein.

68. The cell of any one of claims 62-67, wherein the at least one genetic element is at least one stop codon.

69. The cell of any one of claims 62-68, wherein the first engineered nucleic acid construct is located genomically.

70. A method, comprising:
(a) providing cells that comprise a first engineered nucleic acid construct comprising a first inducible promoter operably linked to a first nucleic acid encoding a reporter protein containing at least one genetic element that prevents transcription of the reporter protein; and
(b) delivering to the cells a second engineered nucleic acid construct comprising a second inducible promoter operably linked to a second nucleic acid that comprises
(i) a nucleotide sequence encoding a single-stranded msr RNA,
(ii) a nucleotide sequence encoding a single-stranded msd DNA modified to contain a targeting sequence complementary to the at least one genetic element that prevents transcription of the reporter protein, and
(iii) optionally a nucleotide sequence encoding a reverse transcriptase protein, wherein (i) and (ii) are flanked by inverted repeat sequences.

71. The method of claim 70, wherein the nucleotide sequence of (i) is upstream of the nucleotide sequence of (ii), which is upstream of the nucleotide sequence of (iii).

72. The method of claim 70 or 71, wherein the method further comprises delivering to the cells an engineered nucleic acid construct that comprises a promoter operably linked to a nucleic acid encoding a single-stranded DNA (ssDNA)-annealing recombinase protein.

73. The method of claim 72, wherein the ssDNA-annealing recombinase protein is a Beta recombinase protein or a Beta recombinase protein homolog.

74. The method of claim 70 or 71, wherein the second nucleic acid further comprises a nucleotide sequence encoding a ssDNA-annealing recombinase protein.

75. The method of claim 74, wherein the ssDNA-annealing recombinase protein is a Beta recombinase protein or a Beta recombinase protein homolog.

76. The method of claim 74 or 75, wherein the nucleotide sequence of (i) is upstream of the nucleotide sequence of (ii), which is upstream of the nucleotide sequence of (iii), which is upstream of the nucleotide sequence encoding a ssDNA-annealing recombinase protein.

77. The method of any one of claims 70-76, further comprising exposing the cells to a first signal that regulates transcription of the first nucleic acid and a second signal that regulates transcription of the second nucleic acid.

78. The method of claim 77, wherein the cells are exposed to the second signal under conditions that permit transcription of the second nucleic acid and recombination of the targeting sequence, and then the cells are exposed to the first signal under conditions that permit transcription of the first nucleic acid.

79. The method of claim 77, wherein the cells are exposed to the second signal under conditions that permit transcription of the second nucleic acid and recombination of the targeting sequence, exposure of the cells to the second signal is discontinued, and then the cells are exposed to the first signal under conditions that permit transcription of the first nucleic acid.

80. The method of claim any one of claims 70-79, further comprising calculating a recombination rate between the targeting sequence and the at least one genetic element.

81. The method of any one of claims 70-80, wherein the at least one genetic element is at least one stop codon.

82. The method of any one of claims 70-81, wherein the first engineered nucleic acid construct is located genomically.

83. A cell, comprising:
(a) a first engineered nucleic acid construct comprising a first inducible promoter operably linked to a first nucleic acid encoding a reporter protein containing at least one genetic element that prevents translation of the reporter protein;
(b) a second engineered nucleic acid construct comprising a second inducible promoter operably linked to a second nucleic acid that comprises
(i) a nucleotide sequence encoding a single-stranded msr RNA,
(ii) a nucleotide sequence encoding a single-stranded msd DNA modified to contain a targeting sequence that is complementary to the at least one genetic element that prevents translation of the reporter protein, and
(iii) optionally a nucleotide sequence encoding a reverse transcriptase protein, wherein (i) and (ii) are flanked by inverted repeat sequences; and
(c) a third engineered nucleic acid construct comprising a third inducible promoter operably linked to a third nucleic acid encoding a single-stranded DNA (ssDNA)-annealing recombinase protein.

84. The cell of claim 83, wherein the ssDNA-annealing recombinase protein is a Beta recombinase protein or a Beta recombinase protein homolog.

85. The cell of claim 83 or 84, wherein the at least one genetic element is at least one stop codon.

86. The cell of any one of claims 83-85, wherein the first engineered nucleic acid construct is located genomically.

87. The cell of any one of claims 83-86, wherein the nucleotide sequence of (i) is upstream of the nucleotide sequence of (ii), which is upstream of the nucleotide sequence of (iii).

88. A method, comprising:
(a) providing cells that comprise a first engineered nucleic acid construct comprising a first inducible promoter operably linked to a first nucleic acid encoding a reporter protein containing at least one genetic element that prevents translation of the reporter protein; and
(b) delivering to the cells a second engineered nucleic acid construct comprising a second inducible promoter operably linked to a second nucleic acid that comprises
(i) a nucleotide sequence encoding a single-stranded msr RNA,
(ii) a nucleotide sequence encoding a single-stranded msd DNA modified to contain a targeting sequence that is complementary to the at least one genetic element that prevents translation of the reporter protein, and
(iii) optionally a nucleotide sequence encoding a reverse transcriptase protein, wherein (i) and (ii) are flanked by inverted repeat sequences.

89. The method of claim 88, further comprising delivering to the cells a third engineered nucleic acid construct comprising a third inducible promoter operably linked to a third nucleic acid encoding a single-stranded DNA (ssDNA)-annealing recombinase protein.

90. The method of claim 89, wherein the ssDNA-annealing recombinase protein is a Beta recombinase protein or a Beta recombinase protein homolog.

91. The method of claim 89 or 90, further comprising exposing the cells to a first signal that regulates transcription of the first nucleic acid, a second signal that regulates transcription of the second nucleic acid, and a third signal that regulates transcription of the third nucleic acid.

92. The method of claim 91, wherein the cells are exposed to the second and third signal under conditions that permit transcription of the second and third nucleic acids, respectively, and recombination of the targeting sequence, and then the cells are exposed to the first signal under conditions that permit transcription of the first nucleic acid.

93. The method of claim 91 or 92, further comprising calculating a recombination rate between the targeting sequence and the at least one genetic element.

94. The method of any one of claims 88-93, wherein the at least one genetic element is at least one stop codon.

95. The method of any one of claims 88-94, wherein the first engineered nucleic acid construct is located genomically.

96. A method of performing multiplex automated genome editing, comprising:
(a) delivering to cells having a genome at least one of the engineered nucleic acid constructs of any one of claims 1-7, and
(b) culturing the cells under conditions suitable for nucleic acid expression and integration of the single-stranded msd DNA into the genome of cells of (a).

97. A method of producing a nucleic acid nanostructure comprising
(a) delivering to cells a plurality of the engineered nucleic acid constructs of any one of claims 1-7, wherein single-stranded msd DNAs are designed to self-assemble through complementary nucleotide base-pairing into a nucleic acid nanostructure; and
(b) culturing the cells under conditions suitable for nucleic acid expression and self-assembly.

98. The method of claim 97, wherein the nucleic acid nanostructure is a two-dimensional or a three-dimensional nucleic acid nanostructure.

99. The method of claim 97 or 98, wherein the nucleic acid nanostructure is a nucleic acid nanorobot.
